(19) United States
(12) Patent Application Publication
(10) Pub. No.: US 2024/0182983 A1
(43) Pub. Date: Jun. 6, 2024

(54) CELL-FREE DNA METHYLATION TEST

(71) Applicant: UNIVERSITY OF SOUTHERN CALIFORNIA, Los Angeles, CA (US)

(72) Inventors: Budur SALHIA, Los Angeles, CA (US); Gerald Christopher GOODEN, Los Angeles, CA (US)

(73) Assignee: UNIVERSITY OF SOUTHERN CALIFORNIA, Los Angeles, CA (US)

(21) Appl. No.: 18/546,472

(22) PCT Filed: Feb. 17, 2022
(86) PCT No.: PCT/US22/16769

§ 371 Date: Aug. 15, 2023

Related U.S. Application Data
(60) Provisional application No. 63/150,207, filed on Feb. 17, 2021.

(51) Int. Cl. C12Q 1/6886; G16B 20/20; G16B 40/00; G16H 50/20; G16H 50/30

(52) U.S. Cl. CPC C12Q 1/6886; G16B 20/20; G16B 40/00; G16H 50/20; G16H 50/30; C12Q 2600/112; C12Q 2600/118; C12Q 2600/154

(57) ABSTRACT

The disclosure provides for certain assays and methods of determining the presence or absence of ovarian cancer, the severity of ovarian cancer, the histological subtype of ovarian cancer, or the susceptibility to ovarian cancer by examining the methylation levels of certain target genomic regions.

### RELATED APPLICATIONS

This application claims priority under 35 U.S.C. § 119(e) to U.S. Provisional Patent Application No. 63/150,207 filed Feb. 17, 2021, which is incorporated herein by reference in its entirety.

### BACKGROUND OF THE INVENTION

Epithelial ovarian cancer (EOC) is the most lethal gynecologic malignancy with a 5-year survival rate under 50%. Histological subtypes of EOC include endometrioid, mucinous, clear cell and serous. Of these, high-grade serous ovarian cancer (HGSOC) is the most common subtype. Clinically it is the most aggressive and often presents at a later stage compared with other subtypes. Of the 22,240 expected new cases of ovarian cancer in 2020, 75% of these patients will present with advanced stage, where a cure is unlikely, and recurrence is common. In contrast, only 15% of women will present with stage 1 cancer, where the disease is confined to the ovary, and the 5-year survival rate is over 90%.

Studies have shown that patients with ovarian cancer who are operated on by gynecologic oncologists with previous training in cytoreductive techniques are more likely to have better surgical staging, achieve a higher rate of complete cytoreduction in advanced stages and have better overall outcomes in comparison with those patients treated by general gynecologists or general surgeons. However, the access and referral to gynecologic oncologists for women with suspected gynecological cancer is scarce. Therefore, a major impediment to appropriate referral patterns is the challenge of identifying which subgroup of women with a pelvic mass is most likely to have EOC. The cancer antigen 125 test (CA125) is currently utilized as a marker of EOC. However, it is non-specific, with high false positive rates and is elevated in many different conditions, including menstruation, pregnancy, uterine fibroids, endometriosis, appendicitis and other malignancies. Many attempts have been made to improve the specificity of CA125. Approaches have included adding other serum proteins, such as beta2 microglobulin in the OVA1 test (Vermillion labs) or adding transvaginal ultrasonography for ovarian assessment (Risk of Malignancy Index). Nonetheless, these serum protein and imaging-based approaches have largely been inadequate as they have not yielded a shift in the diagnosis of EOC, especially at the earlier stages. In addition, they lack the sensitivity and specificity to be used for screening.

Accordingly, there is a need for a new method of discriminating EOC from benign pelvic masses and for screening for EOC in asymptomatic women that is more sensitive and has higher specificity than previous methods. The present disclosure satisfies these needs.

### SUMMARY OF THE INVENTION

Women who develop pelvic masses face the fear and uncertainty of ovarian cancer. Every year tens of thousands of women undergo surgery to remove pelvic masses—the only way to confirm ovarian cancer. Many surgeries may be unnecessary or delayed, as 80% of pelvic masses are benign. Additionally, most women with EOC are not referred to a gynecologic oncologists, which is needed for patients to get the proper surgical management of EOC, including applying proper cytoreductive techniques that leads to better overall outcomes. With current diagnostic criteria, a major challenge to proper referral of a women for surgery is identifying which subgroup with a pelvic mass is most likely to have ovarian cancer and benefit from surgery. The cancer antigen 125 test (CA125) is currently utilized as a marker of ovarian cancer. However, it is non-specific, with high false positive rates (especially in early stages when cancer is curable) and is elevated in many different conditions, including uterine fibroids and endometriosis.

The ability to distinguish benign from malignant pelvic masses preoperatively, and detecting EOC in asymptomatic women, especially at early stages, is of significant clinical benefit. To solve this problem, a minimally invasive tumor-specific cell-free (cf)DNA methylation test was designed to diagnose ovarian cancer preoperatively and definitively in women with a known pelvic mass by measuring DNA methylation levels of certain genes as an indication of tumorigenicity. DNA methylation is a centrally important modification for the maintenance of large genomes. There are several advantages to utilizing aberrant DNA methylation over other molecular alterations such as point mutations or serum-based protein markers. First, DNA methylation changes occur early in tumorigenesis and are highly chemically stable marks. Second, enhanced detection sensitivity of aberrantly methylated DNA is afforded by its frequency and distribution. Third, DNA methylation measurements incorporate numerous regions, each with multiple CpG positions, allowing better limits of detection than for protein-based markers or DNA mutations. Fourth, aberrant CpG island hypermethylation rarely occurs in normal cells. Therefore, the DNA methylation signal can be detected with a notable degree of sensitivity, even in the presence of background methylation derived from normal cells. Fifth, large-scale DNA methylation alterations are tissue- and cancer-type specific and therefore potentially have greater ability to detect and classify cancers in patients with early-stage disease. The development and implementation of this liquid biopsy assay fills the void of a clinically unmet need and would greatly enhance EOC screening and diagnosis. Thus, this disclosure will give doctors the tools they need to appropriately select women with pelvic masses for surgery.

Accordingly, the disclosure provides for embodiments for determining the likelihood of having or developing epithelial ovarian cancer, the presence or absence of epithelial ovarian cancer, determining the presence of high grade serous epithelial ovarian cancer, determine the severity of epithelial ovarian cancer, determine the histological subtype of the epithelial ovarian cancer, differentiate between high grade serous epithelial ovarian cancer and non-high grade serous epithelial ovarian cancer.

In one embodiment, a method for determining whether a subject is likely to have or develop epithelial ovarian cancer in a subject comprising: measuring the level of nucleic acid methylation of a plurality of target genomic region listed in Table 1 from a cell-free nucleic acid sample from the subject; comparing the level of nucleic acid methylation of the plurality of target genomic region in the sample to the level of nucleic acid methylation of the plurality of target genomic regions in a sample isolated from a cancer-free subject, a cancer-free reference standard, or a cancer-free reference cutoff value; determining that the subject is like to have or develop epithelial ovarian cancer based on a change in the level of nucleic acid methylation in the plurality of target genomic regions in the sample derived from the subject, wherein the change is greater or lower than the level of nucleic acid methylation of the target genomic regions in the sample isolated from a cancer-free subject, a normal reference standard, or a normal reference cutoff value.

In some embodiments, the method determines a presence of stage 1, stage II, stage III, or stage IV epithelial ovarian cancer of any epithelial histological subtype. In some embodiments, the epithelial histological subtype is selected from the group consisting of endometrioid ovarian cancer, mucinous ovarian cancer, clear cell ovarian cancer, and serous ovarian cancer.

In some embodiments, the methylation level is determined using one or more of enzymatic treatment, bisulfite amplicon sequencing (BSAS), bisulfite treatment of DNA, methylation sensitive PCR, bisulfite conversion combined with bisulfite restriction analysis, post whole genome library hybrid probe capture, and TRollCamp sequencing.

In some embodiments, the methylation level of the target genomic regions is determined using hybrid probe capture. Hybrid prob capture may comprise one or more probes that hybridize to the one or more target genomic regions, wherein the one or more target genomic regions comprise an uracil at each position corresponding to an unmethylated cytosine in the DNA molecule. The probes can be configured to hybridize to: a) a nucleotide sequence of the one or more target genomic regions comprising uracil at each position corresponding to a cytosine of a CpG site of the nucleic acid molecule; or b) a nucleotide sequence of the one or more target genomic regions comprising cytosine at each position corresponding to a cytosine of a CpG site of the nucleic acid molecule.

In some embodiments, the hybrid capture probes comprise ribonucleic acid, and each of the probes also may comprise and affinity tag such as biotin or streptavidin.

In some embodiments, the plurality of target genomic regions comprises at least 10%, at least 20%, at least 30%, at least 40%, at least 50%, at least 60%, at least 70%, at least 80%, at least 90%, at least 95% or greater than 95% of the target genomic regions listed in Table 1.

In some embodiments, the plurality of target genomic regions excludes the genomic target regions Chr2: 38323997-38324203, Chr2: 113712408-113712611, Chr3:20029245-20029704, Chr8:58146211-58146673, Chr8:124995553-124995624, Chr9:89438825-89439085, Chr11:63664463-63664769, Chr11:120496972-120497256, and Chr20:5452392-5452552.

In some embodiments, the methods disclosed herein further comprising treating the epithelial ovarian cancer in the subject, wherein the treatment comprises one or more of radiation therapy, surgery to remove the cancer and, administering a therapeutic agent to the patient.

In some embodiments, a trained machine learning algorithm is used to determine whether the subject is likely to have or develop the epithelial ovarian cancer, the presence or absence of epithelial ovarian cancer, determining the presence of high grade serous epithelial ovarian cancer, determine the severity of epithelial ovarian cancer, determine the histological subtype of the epithelial ovarian cancer, differentiate between high grade serous epithelial ovarian cancer and non-high grade serous epithelial ovarian cancer.

In some embodiments, the machine learning algorithm comprises a Random Forest, a support vector machine (SVM), a neural network, or a deep learning algorithm.

In some embodiments, the trained machine learning algorithm is trained using samples comprising known epithelial ovarian cancer samples and known cancer-free ovarian and/or fallopian tubes samples and the target genomic regions listed in Table 1 are examined to train the algorithm.

These and other features and advantages of this invention will be more fully understood from the following detailed description of the invention taken together with the accompanying claims. It is noted that the scope of the claims is defined by the recitations therein and not by the specific discussion of features and advantages set forth in the present description.

### BRIEF DESCRIPTION OF THE DRAWINGS

The following drawings form part of the specification and are included to further demonstrate certain embodiments or various aspects of the invention. In some instances, embodiments of the invention can be best understood by referring to the accompanying drawings in combination with the detailed description presented herein. The description and accompanying drawings may highlight a certain specific example, or a certain aspect of the invention. However, one skilled in the art will understand that portions of the example or aspect may be used in combination with other examples or aspects of the invention.

FIG. 1. Dimensionality reduction using uniform manifold approximation and projection (UMAP), a form of multidimensional scaling (MDS), which simplifies multivariate data to a 2-dimensional plane. The UMAP visually shows how separable the classes under consideration are with respect to the selected group of features. It is a 2D plot and represents each class as a cluster of points in a unique shape. Each point represents one samples' methylation profile from reduced representation bisulfite sequencing (RRBS). The UMAP was generated from average (mean) beta values extracted from each RRBS sample across the 1677 regions identified by DMR analysis.

FIG. 2. Classifier model built from cfDNA methylation levels of select DMRs predicts ovarian cancer disease status. (A) DNA methylation values of plasma cfDNA were assayed in 35 amplicons. The samples were randomly split into training (70%) and testing (30%) datasets for machine learning classification. C5.0 decision tree algorithm was used to build a predictive model from the training dataset. The model was then used to predict probability of having ovarian cancer in the testing set. Dot plots show the aggregated predictions from both training and testing sets based on stage. The final model utilized 20/35 of the selected regions. 2/4 of the samples were false positives that did not classify correctly (circled red) had either a history of other cancers or developed them later on in time. (B) The 2 false positive samples were dropped and the classifier model was rebuilt. The dot plot shows the new predictions from the updated model. 2_8_GTFR_632-54yo with 34 cm mucinous cystadenoma (2013), interestingly also with VIN3 at that time (of sample acquisition in 2013) and developed stage IA SCC vulva by 2017, currently NED. 1a_65_139369A3_Dx-Benign—53yo serous cystadenoma (size not included) but on looking at the original information sheet she has a history of “malignant neoplasm of the uterus” and reported chemo meds in the med list.

FIG. 3. Performance metrics of classifier model shows high accuracy of prediction. Receiver operating characteristic (ROC) curve and performance metrics of the classifier model run on plasma cfDNA. ROC curve and metrics were derived from predictions of the either (A) the initial model containing all samples or (B) the updated model with the 2 false positive samples removed. Area under the curve (AOC) calculated from the ROC curve was high, indicating our model is a strong predictor for ovarian cancer status. Abbreviations: PPV—positive predictive value; NPV—negative predictive value.

FIG. 4. Reproducibility of bisulfite amplicon sequencing (A) and hybrid probe capture (B). A) Scatterplot of bisulfite amplicon sequencing data displaying the correlation of the average methylation (beta) levels of each region across two biological replicates in two different samples (top and bottom panels). Replicates show high correlation, with Pearson correlation equal to 0.99 B) Scatter plots comparing samples captured multiple times. Hybrid probe capture shows high beta value consistency between different captures (x and y). R² values are high indicating high reproducibility between different captures in 8 different samples represented (each panel is a unique sample).

### DETAILED DESCRIPTION OF THE INVENTION

### Definitions

The following definitions are included to provide a clear and consistent understanding of the specification and claims. As used herein, the recited terms have the following meanings. All other terms and phrases used in this specification have their ordinary meanings as one of skill in the art would understand. Such ordinary meanings may be obtained by reference to technical dictionaries, such as 14^{th} Edition, by R. J. Lewis, John Wiley & Sons, New York, N.Y., 2001.

References in the specification to “one embodiment”, “an embodiment”, etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The phrases “one or more” and “at least one” are readily understood by one of skill in the art, particularly when read in context of its usage. For example, the phrase can mean one, two, three, four, five, six, ten, 100, or any upper limit approximately 10, 100, or 1000 times higher than a recited lower limit. For example, one or more substituents on a phenyl ring refers to one to five substituents on the ring.

As will be understood by the skilled artisan, all numbers, including those expressing quantities of ingredients, properties such as molecular weight, reaction conditions, and so forth, are approximations and are understood as being optionally modified in all instances by the term “about.” These values can vary depending upon the desired properties sought to be obtained by those skilled in the art utilizing the teachings of the descriptions herein. It is also understood that such values inherently contain variability necessarily resulting from the standard deviations found in their respective testing measurements. When values are expressed as approximations, by use of the antecedent “about,” it will be understood that the particular value without the modifier “about” also forms a further aspect.

The terms “about” and “approximately” are used interchangeably. Both terms can refer to a variation of ±5%, ±10%, ±20%, or ±25% of the value specified. For example, “about 50” percent can in some embodiments carry a variation from 45 to 55 percent, or as otherwise defined by a particular claim. For integer ranges, the term “about” can include one or two integers greater than and/or less than a recited integer at each end of the range. Unless indicated otherwise herein, the terms “about” and “approximately” are intended to include values, e.g., weight percentages, proximate to the recited range that are equivalent in terms of the functionality of the individual ingredient, composition, or embodiment. The terms “about” and “approximately” can also modify the endpoints of a recited range as discussed above in this paragraph.

As will be understood by one skilled in the art, for any and all purposes, particularly in terms of providing a written description, all ranges recited herein also encompass any and all possible sub-ranges and combinations of sub-ranges thereof, as well as the individual values making up the range, particularly integer values. It is therefore understood that each unit between two particular units are also disclosed. For example, if 10 to 15 is disclosed, then 11, 12, 13, and 14 are also disclosed, individually, and as part of a range. A recited range (e.g., weight percentages or carbon groups) includes each specific value, integer, decimal, or identity within the range. Any listed range can be easily recognized as sufficiently describing and enabling the same range being broken down into at least equal halves, thirds, quarters, fifths, or tenths. As a non-limiting example, each range discussed herein can be readily broken down into a lower third, middle third and upper third, etc. As will also be understood by one skilled in the art, all language such as “up to”, “at least”, “greater than”, “less than”, “more than”, “or more”, and the like, include the number recited and such terms refer to ranges that can be subsequently broken down into sub-ranges as discussed above. In the same manner, all ratios recited herein also include all sub-ratios falling within the broader ratio. Accordingly, specific values recited for radicals, substituents, and ranges, are for illustration only; they do not exclude other defined values or other values within defined ranges for radicals and substituents. It will be further understood that the endpoints of each of the ranges are significant both in relation to the other endpoint, and independently of the other endpoint.

This disclosure provides ranges, limits, and deviations to variables such as volume, mass, percentages, ratios, etc. It is understood that a range, such as “number 1” to “number 2”, implies a continuous range of numbers that includes the whole numbers and fractional numbers. For example, 1 to 10 means 1, 2, 3, 4, 5, . . . 9, 10. It also means 1.0, 1.1, 1.2. 1.3, . . . , 9.8, 9.9, 10.0, and also means 1.01, 1.02, 1.03, and so on. If the variable disclosed is a number less than “number10”, it implies a continuous range that includes whole numbers and fractional numbers less than number10, as discussed above. Similarly, if the variable disclosed is a number greater than “number10”, it implies a continuous range that includes whole numbers and fractional numbers greater than number10. These ranges can be modified by the term “about”, whose meaning has been described above.

One skilled in the art will also readily recognize that where members are grouped together in a common manner, such as in a Markush group, the invention encompasses not only the entire group listed as a whole, but each member of the group individually and all possible subgroups of the main group. Additionally, for all purposes, the invention encompasses not only the main group, but also the main group absent one or more of the group members. The invention therefore envisages the explicit exclusion of any one or more of members of a recited group. Accordingly, provisos may apply to any of the disclosed categories or embodiments whereby any one or more of the recited elements, species, or embodiments, may be excluded from such categories or embodiments, for example, for use in an explicit negative limitation.

The term “contacting” refers to the act of touching, making contact, or of bringing to immediate or close proximity, including at the cellular or molecular level, for example, to bring about a physiological reaction, a chemical reaction, or a physical change, e.g., in a solution, in a reaction mixture, in vitro, or in vivo.

An “effective amount” refers to an amount effective to treat a disease, disorder, and/or condition, or to bring about a recited effect. For example, an effective amount can be an amount effective to reduce the progression or severity of the condition or symptoms being treated. Determination of a therapeutically effective amount is well within the capacity of persons skilled in the art. The term “effective amount” is intended to include an amount of a compound described herein, or an amount of a combination of compounds described herein, e.g., that is effective to treat or prevent a disease or disorder, or to treat the symptoms of the disease or disorder, in a host. Thus, an “effective amount” generally means an amount that provides the desired effect.

Alternatively, the terms “effective amount” or “therapeutically effective amount,” as used herein, refer to a sufficient amount of an agent or a composition or combination of compositions being administered which will relieve to some extent one or more of the symptoms of the disease or condition being treated. The result can be reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case may be determined using techniques, such as a dose escalation study. The dose could be administered in one or more administrations. However, the precise determination of what would be considered an effective dose may be based on factors individual to each patient, including, but not limited to, the patient's age, size, type or extent of disease, stage of the disease, route of administration of the compositions, the type or extent of supplemental therapy used, ongoing disease process and type of treatment desired (e.g., aggressive vs. conventional treatment).

The terms “treating”, “treat” and “treatment” include (i) preventing a disease, pathologic or medical condition from occurring (e.g., prophylaxis); (ii) inhibiting the disease, pathologic or medical condition or arresting its development; (iii) relieving the disease, pathologic or medical condition; and/or (iv) diminishing symptoms associated with the disease, pathologic or medical condition. Thus, the terms “treat”, “treatment”, and “treating” can extend to prophylaxis and can include prevent, prevention, preventing, lowering, stopping, or reversing the progression or severity of the condition or symptoms being treated. As such, the term “treatment” can include medical, therapeutic, and/or prophylactic administration, as appropriate.

As used herein, “subject” or “patient” means an individual having symptoms of, or at risk for, a disease or other malignancy. A patient may be human or non-human and may include, for example, animal strains or species used as “model systems” for research purposes, such a mouse model as described herein. Likewise, patient may include either adults or juveniles (e.g., children). Moreover, patient may mean any living organism, preferably a mammal (e.g., human or non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish, and the like. In one embodiment of the methods provided herein, the mammal is a human.

As used herein, the terms “providing”, “administering,” “introducing,” are used interchangeably herein and refer to the placement of a compound of the disclosure into a subject by a method or route that results in at least partial localization of the compound to a desired site. The compound can be administered by any appropriate route that results in delivery to a desired location in the subject.

The terms “inhibit”, “inhibiting”, and “inhibition” refer to the slowing, halting, or reversing the growth or progression of a disease, infection, condition, or group of cells. The inhibition can be greater than about 20%, 40%, 60%, 80%, 90%, 95%, or 99%, for example, compared to the growth or progression that occurs in the absence of the treatment or contacting.

The term “gene” refers to a polynucleotide containing at least one open reading frame (ORF) that can be transcribed into an RNA (e.g., miRNA, siRNA, mRNA, tRNA, and rRNA) that may encode a particular polypeptide or protein after being transcribed and translated. Any of the polynucleotide or polypeptide sequences described herein may be used to identify larger fragments or full-length coding sequences of the gene with which they are associated. Methods of isolating larger fragment sequences are known to those of skill in the art.

The term “asymptomatic” refers to a subject that has epithelial ovarian cancer or malignant tumor but is unaware of the presence of the epithelial ovarian cancer or the malignant tumor, or a subject that does not have epithelial ovarian cancer but will develop the epithelial ovarian cancer in the future.

The term “amplicon” refers to nucleic acid products resulting from the amplification of a target nucleic acid sequence. Amplification is often performed by PCR. Amplicons can range in size from 20 base pairs to 15000 base pairs in the case of long-range PCR but are more commonly 100-1000 base pairs for bisulfite-treated DNA used for methylation analysis.

The term “amplification” refers to an increase in the number of copies of a nucleic acid molecule. The resulting amplification products are called “amplicons.” Amplification of a nucleic acid molecule (such as a DNA or RNA molecule) refers to use of a technique that increases the number of copies of a nucleic acid molecule in a sample. An example of amplification is the polymerase chain reaction (PCR), in which a sample is contacted with a pair of oligonucleotide primers under conditions that allow for the hybridization of the primers to a nucleic acid template in the sample. The product of amplification can be characterized by such techniques as electrophoresis, restriction endonuclease cleavage patterns, oligonucleotide hybridization or ligation, and/or nucleic acid sequencing. In some embodiments, the methods provided herein can include a step of producing an amplified nucleic acid under isothermal or thermal variable conditions.

The term “biological sample” refers to a sample obtained from an individual. As used herein, biological samples include all clinical samples containing genomic DNA (such as cell-free genomic DNA) useful for cancer diagnosis and prognosis, including, but not limited to, cells, tissues, and bodily fluids, such as: blood, derivatives and fractions of blood (such as serum or plasma), buccal epithelium, saliva, urine, stools, bronchial aspirates, sputum, biopsy (such as tumor biopsy), and CVS samples. A “biological sample” obtained or derived from an individual includes any such sample that has been processed in any suitable manner (for example, processed to isolate genomic DNA for bisulfite treatment) after being obtained from the individual.

The term “bisulfite treatment” refers to the treatment of DNA with bisulfite or a salt thereof, such as sodium bisulfite (NaHSO₃). Bisulfite reacts readily with the 5,6-double bond of cytosine, but poorly with methylated cytosine. Cytosine reacts with the bisulfite ion to form a sulfonated cytosine reaction intermediate which is susceptible to deamination, giving rise to a sulfonated uracil. The sulfonate group can be removed under alkaline conditions, resulting in the formation of uracil. Uracil is recognized as a thymine by polymerases and amplification will result in an adenine-thymine base pair instead of a cytosine-guanine base pair.

The term “cancer” refers to a biological condition in which a malignant tumor or other neoplasm has undergone characteristic anaplasia with loss of differentiation, increased rate of growth, invasion of surrounding tissue, and which is capable of metastasis. A neoplasm is a new and abnormal growth, particularly a new growth of tissue or cells in which the growth is uncontrolled and progressive. A tumor is an example of a neoplasm. Non-limiting examples of types of cancer include lung cancer, stomach cancer, colon cancer, breast cancer, uterine cancer, bladder, head and neck, kidney, liver, ovarian, pancreas, prostate, and rectal cancer. In some embodiments, the cancer is a type of ovarian cancer, and more particularly, an epithelial ovarian cancer. Exemplary epithelial ovarian cancers include, but not limited to, high-grade serous ovarian cancer (HGSOC), high-grade serous carcinomas, low grade serous carcinomas, primary peritoneal carcinomas, fallopian tube cancer, clear cell carcinomas, endometrioid carcinomas, squamous cell carcinomas, and mucinous carcinomas

The term “DNA (deoxyribonucleic acid)” refers to a long chain polymer which comprises the genetic material of most living organisms. The repeating units in DNA polymers are four different nucleotides, each of which comprises one of the four bases, adenine, guanine, cytosine, and thymine bound to a deoxyribose sugar to which a phosphate group is attached. Triplets of nucleotides (referred to as codons) code for each amino acid in a polypeptide, or for a stop signal. The term codon is also used for the corresponding (and complementary) sequences of three nucleotides in the mRNA into which the DNA sequence is transcribed.

The term “cell-free nucleic acid” or “cell-free polynucleotides” are used interchangeably and refer to any extracellular nucleic acid that is not attached to a cell. A cell-free nucleic acid can be a nucleic acid circulating in blood. Alternatively, a cell-free nucleic acid can be a nucleic acid in other bodily fluid disclosed herein, e.g., urine. A cell-free nucleic acid can be a deoxyribonucleic acid (“DNA”), e.g., genomic DNA, mitochondrial DNA, or a fragment thereof. A cell-free nucleic acid can be a ribonucleic acid (“RNA”), e.g., mRNA, short-interfering RNA (siRNA), microRNA (miRNA), circulating RNA (cRNA), transfer RNA (tRNA), ribosomal RNA (rRNA), small nucleolar RNA (snoRNA), Piwi-interacting RNA (piRNA), long non-coding RNA (long ncRNA), or a fragment thereof. In some cases, a cell-free nucleic acid is a DNA/RNA hybrid. A cell-free nucleic acid can be double-stranded, single-stranded, or a hybrid thereof. A cell-free nucleic acid can be released into bodily fluid through secretion or cell death processes, e.g., cellular necrosis and apoptosis.

A cell-free nucleic acid can comprise one or more epigenetically modifications. For example, a cell-free nucleic acid can be acetylated, methylated, ubiquitylated, phosphorylated, sumoylated, ribosylated, and/or citrullinated. For example, a cell-free nucleic acid can be methylated cell-free DNA.

The term “polynucleotide” refers to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, or EST), exons, introns, messenger RNA (mRNA), transfer RNA, ribosomal RNA, ribozymes, cDNA, RNAi, siRNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any embodiment of this invention that is a polynucleotide encompasses both the double-stranded form and each of two complementary single-stranded forms known or predicted to make up the double-stranded form. A polynucleotide is composed of a specific sequence of four nucleotide bases: adenine (A); cytosine (C); guanine (G); thymine (T); and uracil (U) for thymine when the polynucleotide is RNA. Thus, the term “polynucleotide sequence” is the alphabetical representation of a polynucleotide molecule. This alphabetical representation can be input into databases in a computer having a central processing unit and used for bioinformatics applications such as functional genomics and homology searching.

The term “methylation level” refers to the state of DNA methylation (methylated or not methylated) of the cytosine nucleotide of one or more CpG sites within a genomic sequence.

The term “CpG island” refers to a region of DNA with a high frequency and/or enrichment of CpG sites. Algorithms can be used to identify CpG islands (Han, L. et al. (2008) Genome Biology, 9(5): R79). Generally, enrichment is defined as a ratio of observed-to-expected CpGs for a given DNA sequence greater than about 40%, about 50%, about 60%, about 70%, about 80%, or about 90-100%. The term “CpG Site” refers to a di-nucleotide DNA sequence comprising a cytosine followed by a guanine in the 5′ to 3′ direction. The cytosine nucleotides of CpG sites in genomic DNA are the target of intracellular methyltransferases and can have a methylation status of methylated or not methylated. Reference to “methylated CpG site” or similar language refers to a CpG site in genomic DNA having a 5-methylcytosine nucleotide.

“Homology” or “identity” or “similarity” are synonymously and refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, or alternatively less than 25% identity, with one of the sequences of the present invention.

A polynucleotide or polynucleotide region (or a polypeptide or polypeptide region) has a certain percentage (for example, 60%, 65%, 70%, 75%, 80%, 85%, 90%, 95%, 98% or 99%) of “sequence identity” to another sequence means that, when aligned, that percentage of bases (or amino acids) are the same in comparing the two sequences. This alignment and the percent homology or sequence identity can be determined using software programs known in the art, for example those described in Ausubel et al. eds. (2007) Current Protocols in Molecular Biology. Preferably, default parameters are used for alignment. One alignment program is BLAST, using default parameters. In particular, programs are BLASTN and BLASTP, using the following default parameters: Genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+SwissProtein+SPupdate+PIR. Details of these programs can be found at the following Internet address: www.ncbi.nlm.nih.goviblast/Blast.cgi. Biologically equivalent polynucleotides are those having the specified percent homology and encoding a polypeptide having the same or similar biological activity.

The term “complement” as used herein means the complementary sequence to a nucleic acid according to standard Watson/Crick base pairing rules. A complement sequence can also be a sequence of RNA complementary to the DNA sequence or its complement sequence and can also be a cDNA. The term “substantially complementary” as used herein means that two sequences hybridize under stringent hybridization conditions. The skilled artisan will understand that substantially complementary sequences need not hybridize along their entire length. In particular, substantially complementary sequences comprise a contiguous sequence of bases that do not hybridize to a target or marker sequence, positioned 3′ or 5′ to a contiguous sequence of bases that hybridize under stringent hybridization conditions to a target or marker sequence.

“Hybridization” refers to a reaction in which one or more polynucleotides react to form a complex that is stabilized via hydrogen bonding between the bases of the nucleotide residues. The hydrogen bonding may occur by Watson-Crick base pairing, Hoogstein binding, or in any other sequence-specific manner. The complex may comprise two strands forming a duplex structure, three or more strands forming a multi-stranded complex, a single self-hybridizing strand, or any combination of these. A hybridization reaction may constitute a step in a more extensive process, such as the initiation of a PC reaction, or the enzymatic cleavage of a polynucleotide by a ribozyme.

Examples of stringent hybridization conditions include incubation temperatures of about 25° C. to about 37° C.; hybridization buffer concentrations of about 6×SSC to about 10×SSC; formamide concentrations of about 0% to about 25%; and wash solutions from about 4×SSC to about 8×SSC. Examples of moderate hybridization conditions include incubation temperatures of about 40° C. to about 50° C.; buffer concentrations of about 9×SSC to about 2×SSC; formamide concentrations of about 30% to about 50%; and wash solutions of about 5×SSC to about 2×SSC. Examples of high stringency conditions include incubation temperatures of about 55° C. to about 68° C.; buffer concentrations of about 1×SSC to about 0.1×SSC; formamide concentrations of about 55% to about 75%; and wash solutions of about 1×SSC, 0.1×SSC, or deionized water. In general, hybridization incubation times are from 5 minutes to 24 hours, with 1, 2, or more washing steps, and wash incubation times are about 1, 2, or 15 minutes. SSC is 0.15 M NaCl and 15 mM citrate buffer. It is understood that equivalents of SSC using other buffer systems can be employed.

The term “genomic region” refers to a specific locus in a subject's genome. In some embodiments, the size of the genomic region can range from one base pair to 10⁷ base pairs in length. In particular embodiments, the size of the genomic region is between 10 base pairs and 10,000 base pairs.

As used herein, the term “reference genome” refers to any particular known, sequenced or characterized genome, whether partial or complete, of any organism or virus that may be used to reference identified sequences from a subject. Exemplary reference genomes used for human subjects as well as many other organisms are provided in the on-line genome browser hosted by the National Center for Biotechnology Information (“NCBI”) or the University of California, Santa Cruz (UCSC). A “genome” refers to the complete genetic information of an organism or virus, expressed in nucleic acid sequences. As used herein, a reference sequence or reference genome often is an assembled or partially assembled genomic sequence from an individual or multiple individuals. In some embodiments, a reference genome is an assembled or partially assembled genomic sequence from one or more human individuals. The reference genome can be viewed as a representative example of a species' set of genes. In some embodiments, a reference genome comprises sequences assigned to chromosomes. One exemplary human reference genome is GRCh38 (UCSC equivalent: hg38).

As used herein, the term “normal reference standard” intends a control level, degree, or range of DNA methylation at a particular genomic region or gene in a sample that is not associated with cancer. The term “normal reference cutoff value” refers to a control threshold level of DNA methylation at a particular genomic region or gene or a differential methylation value (DMV). In some embodiments, DNA methylation levels enriched above the normal reference cutoff value are associated with having or developing cancer. In some embodiments, DNA methylation levels at or below the normal reference cutoff value are associated with not having or developing cancer.

“Detecting” as used herein refers to determining the presence and/or degree of methylation in a nucleic acid of interest in a sample. Detection does not require the method to provide 100% sensitivity and/or 100% specificity.

The term “substantially” as used herein, is a broad term and is used in its ordinary sense, including, without limitation, being largely but not necessarily wholly that which is specified. For example, the term could refer to a numerical value that may not be 100% the full numerical value. The full numerical value may be less by about 1%, about 2%, about 3%, about 4%, about 5%, about 6%, about 7%, about 8%, about 9%, about 10%, about 15%, or about 20%.

Wherever the term “comprising” is used herein, options are contemplated wherein the terms “consisting of” or “consisting essentially of” are used instead. As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the aspect element. As used herein, “consisting essentially of” does not exclude materials or steps that do not materially affect the basic and novel characteristics of the aspect. In each instance herein any of the terms “comprising”, “consisting essentially of” and “consisting of” may be replaced with either of the other two terms. The disclosure illustratively described herein may be suitably practiced in the absence of any element or elements, limitation, or limitations not specifically disclosed herein.

### Embodiments of the Invention

The disclosure provides for a panel assay and various methods for detecting a change in methylation levels of a target genomic region where the change of methylation levels of a sample for a subject is analyzed using a trained machine learning algorithm that is trained using differentially methylated target genomic regions of cancerous and non-cancerous control samples. The differences in methylation levels of the target genomic sequences of the sample can indicate, for example, the presence or absence of epithelial ovarian cancer, the severity of epithelial ovarian cancer, the histological subtype of epithelial ovarian cancer, the susceptibility to epithelial ovarian cancer, differentiate between high grade serous epithelial ovarian cancer and non-high grade serous epithelial ovarian cancer, differentiate between a benign tumor and epithelial ovarian cancer, and indicate the presence of an epithelial ovarian cancer in an asymptomatic subject or in a subject genetically predisposed to a type of cancer. Generally, embodiments of the disclosure comprise the steps of bisulfite conversion of the nucleic acids from a cell-free nucleic acid sample of a subject using, for example, Reduced Representation Bisulfite Sequencing (RBSS) or hybrid probe capture; next generation sequencing the converted and enriched nucleic acids; collecting the differential methylation pattern data from the targeted genomic regions (e.g., the target genomic regions listed in Table 1); and using a trained machine learning algorithm to determine, for example, the presence or absence of epithelial ovarian cancer, the severity of epithelial ovarian cancer, the histological subtype of epithelial ovarian cancer, or the susceptibility to epithelial ovarian cancer.

In some embodiments, the biological sample containing the DNA or other nucleic acid that may be examined for methylation levels is collected from a patient having, for example, a tumor or a mass or is suspected of having a tumor or mass. Preferably, the biological sample is collected through a standard biopsy or a liquid biopsy and the nucleic acid in the liquid biopsy is tumor/mass derived cell-free nucleic acid (e.g., cell-free DNA). The cell-free nucleic acid may be collected from whole blood, plasma, serum, or urine.

Isolation and extraction of cell-free nucleic acid may be performed through collection of bodily fluids using a variety of techniques. In some cases, collection may comprise aspiration of a bodily fluid from a subject using a syringe. In other cases, collection may comprise pipetting or direct collection of fluid into a collecting vessel.

After collection of bodily fluid, cell-free nucleic acid may be isolated and extracted using a variety of techniques known to a person of ordinary skill in the art. In some cases, cell-free nucleic acid may be isolated, extracted and prepared using commercially available kits such as the Qiagen Qiamp® Circulating Nucleic Acid Kit protocol. In other examples, Qiagen Qubit™ dsDNA HS Assay kit protocol, Agilent™ DNA 1000 kit, or TruSeq™ Sequencing Library Preparation; Low-Throughput (LT) protocol.

Alternatively, cell free nucleic acids may be extracted and isolated by from bodily fluids through a partitioning step in which e.g., cell-free DNAs, as found in solution, are separated from cells and other non-soluble components of the bodily fluid. Partitioning may include, but is not limited to, techniques such as centrifugation or filtration. In other cases, cells may not be partitioned from cell-free DNA first, but rather lysed. For instance, the genomic DNA of intact cells may be partitioned through selective precipitation.

In some embodiments, the method used to determine the methylation level of the one or more target nucleic acids includes methylation sequencing.

For example, the methylation levels of CpG sites within the target genomic regions listed in Table 1 may be detected using DNA methylation sequencing. DNA methylation sequencing can involve, for example, treating DNA from a sample with bisulfite to convert unmethylated cytosine to uracil followed by amplification (such as PCR amplification) of a target nucleic acid within the treated genomic DNA, and sequencing of the resulting amplicon. Sequencing produces nucleotide reads that may be aligned to a genomic reference sequence that may be used to quantitate methylation levels of all the CpGs within an amplicon. Cytosines in non-CpG context may be used to track bisulfite conversion efficiency for each individual sample. The procedure is both time and cost-effective, as multiple samples may be sequenced in parallel using a 96 well plate and generates reproducible measurements of methylation when assayed in independent experiments.

Nucleic acid molecules may be subjected to conditions sufficient to convert unmethylated cytosines in the nucleic acid molecules to uracils (e.g., subsequent to extraction from a sample). For example, the nucleic acid molecules may be subjected to bisulfite processing. Bisulfite treatment of nucleic acid molecules deaminates unmethylated cytosine bases, converting them to uracil bases. This bisulfite conversion process does not deaminate methylated or hydroxymethylated cytosines (e.g., at the 5 position, such as 5 mC or 5 hmC). Nucleic acid molecules may be oxidized prior to undergoing bisulfite conversion to convert hydroxymethylated cytosine (e.g., 5 hmC) to formylcytosine and carboxylcytosine (e.g., 5-formyl cytosine and 5-carboxylcytosine). These oxidized products may be sensitive to bisulfite conversion. Nucleic acid molecules may also be subjected to further processing including other derivatization processes (e.g., to incorporate, modify, and/or delete one or more sequences, tags, or labels). In some cases, functional sequences (e.g., sequencing adapters, flow cell adapters, sequencing primers, etc.) may be added to nucleic acid molecules to facilitate nucleic acid sequencing. Accordingly, derivatives of nucleic acid molecules from a sample may comprise processed nucleic acid molecules including bisulfite-modified nucleic acid molecules, reverse-transcribed nucleic acid molecules, tagged nucleic acid molecules, barcoded nucleic acid molecules, and other modified nucleic acid molecules.

In some embodiments, methylation levels of a target gene(s) or target regions of the gene(s) may be determined using one or more of hybrid probe capture, targeted bisulfite amplicon sequencing, bisulfite DNA treatment, whole genome bisulfite sequencing, bisulfite conversion combined with bisulfite restriction analysis (COBRA), bisulfite PCR, bisulfite modification, bisulfite pyrosequencing, methylated CpG island amplification, CpG binding column based isolation of CpG islands, CpG island arrays with differential methylation hybridization, high performance liquid chromatography, DNA methyltransferase assay, methylation sensitive PCR, cloning differentially methylated sequences, methylation detection following restriction, restriction landmark genomic scanning, methylation sensitive restriction fingerprinting, or Southern blot analysis.

In some embodiments, the method used to determine the methylation level of the one or more target nucleic acids is targeted rolling circle amplicon (TRollCAmp) sequencing. TrollCAmp sequencing is a technique which enhances and improves standard targeted bisulfite amplicon sequencing. It can be used to enhance targeted or genome-wide bisulfite approaches techniques such as Whole Genome Bisulfite Sequencing (WGBS) or Reduced Representation Bisulfite Sequencing (RRBS). Briefly, it encompasses bisulfite conversion, circular ligation, whole genome amplification/Dnase I digestion, multiplex PCR, library preparation, and sequencing.

TRollCAmp sequencing requires no more than 3 ng of input DNA into the bisulfite conversion. TrollCAmp can produce enough amplified product to run over 1000 separate multiplex PCR reactions, generating data on 5,000-20,000 individual amplicons which is vastly superior to other methods. Furthermore, TRollCAmp-seq exhibits a large dynamic range and generates methylation values that more faithfully recapitulate those observed by other methods. Consequently, TRollCAmp-seq is able to pick up small, statistically significant changes which would be lost due to ratio compression exhibited by other methods. Often, biomarkers and disease specific signatures rely on the presence of many small changes; as such, in some instances TRollCAmp is a favorable option for assay development and clinical translation.

Other methods to assay the methylation status of CpG sites can also be used. Numerous DNA methylation detection methods are known in the art, including but not limited to hybrid probe capture (REF), methylation-specific enzyme digestion (Singer-Sam et al., Nucleic Acids Res. 18(3): 687, 1990; Taylor et al., Leukemia 15(4): 583-9, 2001), methylation-specific PCR (MSP or MSPCR) (Herman et al., Proc Natl Acad Sci USA 93(18): 9821-6, 1996), methylation-sensitive single nucleotide primer extension (MS-SnuPE) (Gonzalgo et al., Nucleic Acids Res. 25(12): 2529-31, 1997), restriction landmark genomic scanning (RLGS) (Kawai, Mol Cell Biol. 14(11): 7421-7, 1994; Akama, et al., Cancer Res. 57(15): 3294-9, 1997), whole genome bisulfite sequencing (Frommer et al., Proc Natl Acad Sci USA 89(5): 1827-31, 1992), and differential methylation hybridization (DMH) (Huang et al., Hum Mol Genet. 8(3): 459-70, 1999). In some embodiments, the methylation levels may be determined using one or more DNA methylation sequencing assays with or without bisulfite treatment of DNA.

In one embodiment, Reduced Representation Bisulfite Sequencing is used to measure methylation levels of a target region. Generally, RRBS begins with the treatment of nucleic acid with bisulfite to convert all unmethylated cytosines into uracil, followed by restriction enzyme digestion (for example, by an enzyme that recognizes a site that includes a CG sequence such as MspI) and complete fragment sequencing after coupling with an adapter ligand. The selection of the restriction enzyme enriches the fragments of the dense regions in CpG, reducing the number of redundant sequences that can map multiple positions of the gene during the analysis. Therefore, RRBS reduces the sample complexity of the nucleic acid sample by selecting a subset (e.g., by size selection using preparative gel electrophoresis) of restriction fragments for sequencing. In opposition to the sequencing of the complete genome with bisulfite, each fragment produced by restriction enzyme digestion contains information on DNA methylation for at least one CpG dinucleotide. Therefore, RRBS enriches the sample in promoters, CpG islands, and other genomic characteristics with a high frequency of restriction enzyme cleavage sites in these regions and, thus, provides an assay to assess the methylation status of one or more genomic loci.

A typical protocol for RRBS comprises the steps of digesting a sample of nucleic acid with a restriction enzyme such as Mspl, filling with projections and A-tails, ligating adapters, conversion with bisulfite, and PCR. See, for example, Gu et al. (2010), Nat Methods 7: 133-6; Meissner et al (2005), Nucleic Acids Res. 33: 5868-77.

In another embodiment, a quantitative assay for target amplification and allele-specific real-time serial (QuARTS) is used to evaluate the methylation status. Three reactions are sequentially produced in each QuARTS assay, including amplification (reaction 1) and cleavage of the target probe (reaction 2) in the primary reaction; and FRET cleavage and generation of the fluorescent signal (reaction 3) in the secondary reaction. When the target nucleic acid is amplified with specific primers, a specific detection probe with a fin sequence binds loosely to the amplicon. The presence of the specific invasive oligonucleotide at the site of binding to the target causes cleavage to release the fin sequence by cutting between the detection probe and the fin sequence. The fin sequence is complementary to a non-fork portion of the corresponding FRET cassette. Accordingly, the fin sequence functions as an invasive oligonucleotide of the FRET cassette and makes a cleavage between the fluorophore of the FRET cassette and an inactivator, which produces a fluorescence signal. The splitting reaction can cut multiple probes per target and thus release multiple fluorophores per fin, providing an exponential signal amplification. QuARTS can detect multiple targets in a single reaction well using FRET cassettes with different dyes. See, for example, in Zou et al. (2010) Clin Chem 56: A199; U.S. patent application Ser. Nos. 12/946,737, 12/946,745, and 12/946,752.

In some embodiments, identifying the presence and/or severity of ovarian cancer in a subject may comprise using hybrid capture probes configured to selectively enrich nucleic acid molecules (e.g., DNA or RNA molecules) or sequences thereof. Such probes may be pull-down probes (e.g., bait sets). Selectively enriched nucleic acid molecules or sequences thereof may correspond to one or more genomic regions in the methylation profile of the data set. The presence of particular sequences, modifications (e.g., methylation states), deletions, additions, single nucleotide polymorphisms, copy number variations, or other features in the selectively enriched nucleic acid molecules or sequences thereof may be indicative of a presence and/or severity of an ovarian cancer. The probes may be selective for a subset of certain target genomic regions of Table 1 in the cell-free biological sample and/or for differentially methylated regions (e.g., CpG sites, CpA, sites, CpT sites, and/or CpC sites). The probes may be configured to selectively enrich nucleic acid molecules (e.g., DNA or RNA molecules) or sequences thereof corresponding to a plurality of target nucleic acid of target genomic sequences, such as the subset of the one or more genomic regions in the cell-free biological sample and/or differentially methylated regions (e.g., CpG sites, CpA, sites, CpT sites, and/or CpC sites). The probes may be nucleic acid molecules (e.g., DNA or RNA molecules) having sequence complementarity with target nucleic acid sequences. These nucleic acid molecules may be primers or enrichment sequences. The assaying of the nucleic acid molecules of the sample (e.g., cell-free biological sample) using probes that are selected for target nucleic acid sequences may comprise use of array hybridization, polymerase chain reaction (PCR), or nucleic acid sequencing (e.g., DNA sequencing or RNA sequencing). The number of target nucleic acid sequences selectively enriched using such a scheme may comprise at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 50, at least 100, at least 150, at least 200, at least 300, at least 500, or more than 500 different target nucleic acid sequences of the target genomic regions. Use of such probes for enrichment of target nucleic acids may be termed “hybrid capture.” Use of such hybrid capture probes may take place prior to or after bisulfite conversion (if applicable). Examples of target nucleic acid sequences include those associated with the genomic regions included in Table 1.

In some embodiments, nucleic acid sample may be collected from plasma samples in a subject having or suspected of having an ovarian cancer or having a benign pelvic mass. The extracted nucleic acids are contacted with a bisulfite compound to undergo bisulfite conversion. A genomic library may then be prepared from the bisulfite converted nucleic acids. A portion of the genomic library may then be hybridized with various capture probes in which the capture probes are complementary to one or more DNA strands of a target genomic region or complementary to the target genomic sequence in which the CpG islands and the like are modified because of bisulfite conversion.

Nonlimiting examples of methods for preparing the library include using a transposome-mediated protocol with dual indexing, and/or a kit (e.g., TruSeq Methyl Capture EPIC Library Prep Kit, Illumina, CA, USA, Kapa Hyper Prep Kit (Kapa Biosystems). Adapters such as TruSeq DNA LT adapters (Illumina) can be used for indexing. Sequencing is performed on the library using a sequencer platform (e.g., MiSeq or HiSeq, Illumina).

Preferably, the capture probe is an RNA probe that is complementary to at least a portion of a nucleic acid sequence of a target genomic region or complementary to at least a portion of a nucleic acid sequence of a target genomic region that is modified because of bisulfite conversion. In some embodiments, several capture probes may be used that overlap one or more portions of each target genomic region (i.e., tiling). In this way, numerous capture probes may be used to saturate a target genomic region to ensure enrichment of that target genomic region. Capture probes may be designed using publicly available software or purchased commercially.

In some embodiments, a capture probe may be tagged with an affinity tag such as biotin, streptavidin, digitonin or other tags that are known in the art. After hybridization to target genomic region, the biotinylated capture probes may be “pulled-down” from the library using streptavidin beads or other streptavidin coated surface, thus causing enrichment of the targeted genomic region. In other embodiments, the probes may be immobilized on a solid surface such as a glass microarray slide.

The enriched target genomic region then may be sequenced using next generation sequencing techniques, such as pyrosequencing, single-molecule real-time sequencing, sequencing by synthesis, sequencing by ligation (SOLID sequencing), and nanopore sequencing.

Nucleic acid molecules (e.g., extracted nucleic acid molecules) or derivatives thereof may be subjected to sequencing to provide a plurality of sequencing reads. Sequencing reads may be aligned with and/or analyzed with regard to a reference genome. Based at least in part on sequencing reads, an absolute amount or relative amount of nucleic acid molecules (including an absolute or relative level of methylation within said molecules) corresponding to one or more genomic regions may be measured. Alternatively, sequencing reads may not be used to determine an amount or relative amount of nucleic acid molecules. A data set comprising a genomic profile (e.g., methylation profile) of one or more genomic regions of a sample may be generated based at least in part on sequencing reads. Sequencing reads may be processed to identify differentially methylated target genomic regions such as hypomethylated and/or hypermethylated regions of the one or more genomic regions.

Sequence identification may be performed by sequencing, array hybridization (e.g., Affymetrix), or nucleic acid amplification (e.g., PCR), for example. Sequencing may be performed by any suitable sequencing methods, such as massively parallel sequencing (MPS), paired-end sequencing, high-throughput sequencing, next-generation sequencing (NGS), shotgun sequencing, single-molecule sequencing, nanopore sequencing, nanopore sequencing with direct detection or inference of methylation status, semiconductor sequencing, pyrosequencing, sequencing-by-synthesis (SBS), sequencing-by-ligation, sequencing-by hybridization, and RNA-Seq (Illumina). Sequencing may comprise bisulfite sequencing (BS-Seq), such as whole genome bisulfite sequencing (WGBS) and/or oxidative bisulfite sequencing (oxBS-Seq).

Sequencing and/or preparing a nucleic acid sample for sequencing may comprise performing one or more nucleic acid reactions such as one or more nucleic acid amplification processes (e.g., of DNA or RNA molecules). Nucleic acid amplification may comprise, for example, reverse transcription, primer extension, asymmetric amplification, rolling circle amplification, ligase chain reaction, polymerase chain reaction (PCR), and multiple displacement amplification. Examples of PCR methods include digital PCR (dPCR), emulsion PCR (ePCR), quantitative PCR (qPCR), real-time PCR (RT-PCR), hot start PCR, multiplex PCR, asymmetric PCR, nested PCR, and assembly PCR. A suitable number of rounds of nucleic acid amplification (e.g., PCR, such as qPCR, RT-PCR, dPCR, etc.) may be performed to sufficiently amplify an initial amount of nucleic acid molecule (e.g., DNA molecule) or derivative thereof to a desired input quantity for subsequent sequencing. In some cases, the PCR may be used for global amplification of nucleic acid molecules. This may comprise using adapter sequences that may be first ligated to different molecules followed by PCR amplification using universal primers. PCR may be performed using any of a number of commercial kits, e.g., provided by Life Technologies, Affymetrix, Promega, Qiagen, etc. In other cases, only certain target nucleic acids within a population of nucleic acids may be amplified. Specific primers, possibly in conjunction with adapter ligation, may be used to selectively amplify certain targets for downstream sequencing. In some cases, nested primers may be used to target specific genomic regions. Nucleic acid amplification may comprise targeted amplification of one or more genetic loci, genomic regions, or differentially methylated regions (e.g., CpG sites, CpA, sites, CpT sites, and/or CpC sites), and in particular, the target genomic regions listed in Table 1 (below). In some cases, nucleic acid amplification is performed after bisulfite conversion. Such a procedure may be termed targeted bisulfite amplicon sequencing (TBAS). Nucleic acid amplification may comprise the use of one or more primers, probes, enzymes (e.g., polymerases), buffers, and deoxyribonucleotides. Nucleic acid amplification may be isothermal or may comprise thermal cycling. Thermal cycling may involve changing a temperature associated with various processes of nucleic acid amplification including, for example, initialization, denaturation, annealing, and extension. Sequencing may comprise use of simultaneous reverse transcription (RT) and PCR, such as a OneStep RT-PCR kit protocol by Qiagen, NEB, Thermo Fisher Scientific, or Bio-Rad.

Nucleic acid molecules (e.g., DNA or RNA molecules) or derivatives thereof may be labeled or tagged, e.g., with identifiable tags, to allow for multiplexing of a plurality of samples. For example, every nucleic acid molecule or derivative thereof associated with a given sample or subject may be tagged or labeled (e.g., with a barcode such as a nucleic acid barcode sequence or a fluorescent label). Nucleic acid molecules or derivatives thereof associated with other samples or subjects may be tagged or labels with different tags or labels such that nucleic acid molecules or derivatives thereof may be associated with the sample or subject from which they derive. Such tagging or labeling also facilitates multiplexing such that nucleic acid molecules or derivatives thereof from multiple samples and/or subjects may be analyzed (e.g., sequenced) at the same time. Any number of samples may be multiplexed. For example a multiplexed reaction may contain nucleic acid molecules or derivatives thereof from at least about 2, 3, 4, 5, 6, 7, 8, 9, 10, 11, 12, 13, 14, 15, 16, 17, 18, 19, 20, 25, 30, 35, 40, 45, 50, 55, 60, 65, 70, 75, 80, 85, 90, 95, 100, or more than 100 initial samples. Such samples may be derived from the same or different subjects. For example, a plurality of samples may be tagged with sample barcodes (e.g., nucleic acid barcode sequences) such that each nucleic acid molecule (e.g., DNA molecule) or derivative thereof may be traced back to the sample (and/or the subject) from which the nucleic acid molecule originated. Sample barcodes may permit samples from multiple subject to be differentiated from one another, which may permit sequences in such samples to be identified simultaneously, such as in a pool. Tags, labels, and/or barcodes may be attached to nucleic acid molecules or derivatives thereof by ligation, primer extension, nucleic acid amplification, or another process. In some cases, nucleic acid molecules or derivatives thereof of a particular sample may be tagged, labeled, or barcoded with different tags, labels, or barcodes (e.g., unique molecular identifiers) such that different nucleic acid molecules or derivatives thereof deriving from the same sample may be differentially tagged, labeled, or barcoded. In some cases, nucleic acid molecules or derivatives thereof from a given sample may be labeled with both different labels and identical labels, such that each nucleic acid molecule or derivative thereof associated with the sample includes both a unique label and a shared label.

After subjecting the nucleic acid molecules or derivatives thereof to sequencing, suitable bioinformatics processes may be performed on the sequence reads to generate the data set comprising the methylation profile of one or more genomic regions of the cell-free biological sample. For example, sequence reads may be aligned to one or more reference genomes (e.g., a human genome). The aligned sequence reads may be quantified at one or more genomic loci to generate the data set comprising the methylation profile of one or more genomic regions of the cell-free biological sample. Quantification of sequences may be expressed as un-normalized or normalized values.

In some embodiments, Alignment of bisulfite converted DNA is performed using a software program such as Bismark (Krueger et al. (2011) Bioinformatics, 27(11): 157171). Bismark performs both read mapping and methylation calling in a single step and its output discriminates between cytosines in CpG, CHG and CHH contexts. Bismark is released under the GNU GPLv3+license. The source code is freely available at bioinformatics.bbsrc.ac.uk/projects/bismark/. In some embodiments, differential methylation is calculated for specific loci/regions using, for example, one or more publicly available programs to analyze and/or determine methylation levels or a target polynucleotide region. In some embodiments, the method used to analyze and/or determine methylation levels of a target polynucleotide region include Metilene (Juhling et al., Genome Res., 2016; 26(2): 256-262) or GenomeStudio Software available online from Illumina, Inc. Other methods of determining differentially methylated target polynucleotide regions are described in Hovestadt et al., 2014; Nature, 510(7506), 537-541.

In some embodiments, the target genomic regions that are examined to determine the presence or absence of ovarian cancer in a subject comprise at least 5%, at least 10%, at least 15%, at least 20%, at least 25%, at least 30%, at least 40%, at least 45%, at least 50%, at least 55%, at least 60%, at least 65%, at least 70%, at least 75%, at least 80%, a least 85%, at least 90%, at least 95%, at least 96%, at least 97%, at least 98%, at least 99%, or 100% of the target genomic regions listed in Table 1.

In some embodiments, the target genomic regions that are examined to determine the severity of ovarian cancer (i.e., stage I, stage II, stage III, or stage IV cancer) subject comprise at least 5%, at least 10%, at least 15%, at least 20%, at least 25%, at least 30%, at least 40%, at least 45%, at least 50%, at least 55%, at least 60%, at least 65%, at least 70%, at least 75%, at least 80%, a least 85%, at least 90%, at least 95%, at least 96%, at least 97%, at least 98%, at least 99%, or 100% of the target genomic regions listed in Table 1.

In some embodiments, the target genomic regions that are examined to preoperatively determine if a pelvic mass is cancerous or benign in a subject comprise at least 5%, at least 10%, at least 15%, at least 20%, at least 25%, at least 30%, at least 40%, at least 45%, at least 50%, at least 55%, at least 60%, at least 65%, at least 70%, at least 75%, at least 80%, a least 85%, at least 90%, at least 95%, at least 96%, at least 97%, at least 98%, at least 99%, or 100% of the target genomic regions listed in Table 1.

In some embodiments, the target genomic regions that are examined to identify a histological subtype of an ovarian cancer in a subject comprise at least 5%, at least 10%, at least 15%, at least 20%, at least 25%, at least 30%, at least 40%, at least 45%, at least 50%, at least 55%, at least 60%, at least 65%, at least 70%, at least 75%, at least 80%, a least 85%, at least 90%, at least 95%, at least 96%, at least 97%, at least 98%, at least 99%, or 100% of the target genomic regions listed in Table 1. In some embodiments, the histological subtype comprises or consists of histological endometrioid ovarian cancer, mucinous ovarian cancer, clear cell ovarian cancer, and serous ovarian cancer.

In some embodiments, the target genomic regions that are examined detect high grade serous ovarian cancer in an asymptomatic subject or subjects a high risk (i.e., having a hereditary predisposition for cancer such as, but not limited to, having one or more mutant alleles of BRCA1, BRCA2, RB, P53, APC, PTEN, or strong family history of cancer) of developing cancer comprise at least 5%, at least 10%, at least 15%, at least 20%, at least 25%, at least 30%, at least 40%, at least 45%, at least 50%, at least 55%, at least 60%, at least 65%, at least 70%, at least 75%, at least 80%, a least 85%, at least 90%, at least 95%, at least 96%, at least 97%, at least 98%, at least 99%, or 100% of the target genomic regions listed in Table 1.

In some embodiments, the methods described herein are useful in non-invasive screening of subjects for epithelial ovarian cancers. For example, target genomic regions are used to screen for epithelial ovarian a cancer in a subject having a tumor mass but who is not symptomatic of cancer during an annual doctor's visit. In another embodiment, the methods described here are useful to screen a subject for epithelial ovarian wherein the subject does not have a tumor mas but has an epithelial ovarian cancer below the standard level of detection using standard means known in the art. Screening using the methods described herein are also useful in a subject at high risk of developing cancer due to a genetic predisposition or strong family history of a cancer.

In some embodiments, the target genomic regions that are examined to exclude the presence of high grade serous ovarian cancer in a subject comprise at least 5%, at least 10%, at least 15%, at least 20%, at least 25%, at least 30%, at least 40%, at least 45%, at least 50%, at least 55%, at least 60%, at least 65%, at least 70%, at least 75%, at least 80%, a least 85%, at least 90%, at least 95%, at least 96%, at least 97%, at least 98%, at least 99%, or 100% of the target genomic regions listed in Table 1.

Some embodiments may be used to determine the presence of minimum residual disease. Minimum residual disease is the name given to small numbers of cancer cells that remain in the person during treatment, or after treatment when the patient is in remission. It is the major cause of relapse in cancer.

Target genomic regions that are examined to determine the presence of minimum residual disease in a subject comprise at least 5%, at least 10%, at least 15%, at least 20%, at least 25%, at least 30%, at least 40%, at least 45%, at least 50%, at least 55%, at least 60%, at least 65%, at least 70%, at least 75%, at least 80%, a least 85%, at least 90%, at least 95%, at least 96%, at least 97%, at least 98%, at least 99%, or 100% of the target genomic regions listed in Table 1.

TABLE 1
Target Genomic Regions. Table 1 including the chromosome numbers, start and stop positions, wilcox
p-value, Differentially Methylated Value (DMR Value), and nearest gene provided relative to known
human reference genome hg38, which is available from Genome Refence Consortium with a reference
number GRCh38/hg38, which is incorporated herein in its entirely, and may be accessed at, for
example, www.ncbi.nlm.nih.gov/grc/human or www.ncbi.nlm.nih.gov/genome/tools/remap.

| seqnames | start | end | wilcox_pval | dmr value | nearest_gene |
|---|---|---|---|---|---|
| chr1 | 779699 | 780069 | 1.04116E−09 | −0.430556482 | LOC100288069 |
| chr1 | 898425 | 898599 | 3.44869E−14 | −0.485213747 | LINC02593 |
| chr1 | 977181 | 977262 | 8.96843E−15 | −0.486587014 | PERM1 |
| chr1 | 977852 | 978434 | 1.28517E−14 | −0.416227571 | PERM1 |
| chr1 | 1024627 | 1024990 | 6.9467E−11 | −0.428342183 | AGRN |
| chr1 | 1050707 | 1050891 | 2.57927E−08 | −0.386851261 | AGRN |
| chr1 | 1163406 | 1164272 | 6.19469E−15 | −0.505847363 | MIR200B |
| chr1 | 1304408 | 1304717 | 1.36237E−12 | 0.401067971 | ACAP3 |
| chr1 | 1304523 | 1304717 | 6.19469E−15 | 0.43440229 | ACAP3 |
| chr1 | 1357450 | 1357844 | 3.12829E−07 | 0.355844511 | MXRA8 |
| chr1 | 1418152 | 1418623 | 6.9467E−11 | 0.468171775 | ANKRD65 |
| chr1 | 1462534 | 1462801 | 3.01215E−06 | 0.31555839 | ATAD3C |
| chr1 | 1513540 | 1513952 | 1.70548E−12 | −0.418028077 | ATAD3A |
| chr1 | 1550340 | 1550663 | 3.69832E−16 | 0.392275381 | SSU72 |
| chr1 | 1630331 | 1631309 | 1.47656E−13 | 0.396513317 | MIB2 |
| chr1 | 1758531 | 1759182 | 1.47656E−13 | −0.4191427 | NADK |
| chr1 | 1764263 | 1764643 | 5.32836E−13 | −0.475844439 | NADK |
| chr1 | 2073182 | 2074331 | 9.2458E−17 | 0.489625343 | PRKCZ |
| chr1 | 2116064 | 2116335 | 1.28517E−14 | 0.440433033 | PRKCZ |
| chr1 | 2320052 | 2320314 | 1.62897E−11 | 0.427260308 | MORN1 |
| chr1 | 2847633 | 2847737 | 4.24652E−10 | −0.409524823 | TTC34 |
| chr1 | 3160915 | 3161368 | 1.70548E−12 | −0.40913903 | PRDM16 |
| chr1 | 3188479 | 3188689 | 1.61457E−10 | −0.450133198 | PRDM16 |
| chr1 | 3258772 | 3258834 | 6.32413E−14 | 0.466586376 | PRDM16 |
| chr1 | 3391698 | 3392188 | 4.69687E−14 | 0.430761649 | PRDM16 |
| chr1 | 5142890 | 5143194 | 6.47206E−16 | −0.416496194 | AJAP1 |
| chr1 | 6105187 | 6105437 | 4.16801E−13 | −0.426617184 | CHD5 |
| chr1 | 6111633 | 6111812 | 9.2458E−17 | −0.402542725 | CHD5 |
| chr1 | 6220320 | 6220448 | 1.3454E−11 | −0.510500915 | RNF207 |
| chr1 | 6427121 | 6427652 | 1.08444E−12 | −0.41518851 | ESPN |
| chr1 | 6454195 | 6454290 | 1.7567E−15 | 0.492900238 | ESPN |
| chr1 | 6454795 | 6455753 | 9.2458E−17 | 0.443407196 | ESPN |
| chr1 | 6460537 | 6461304 | 1.28517E−14 | 0.406394922 | TNFRSF25 |
| chr1 | 6961465 | 6961875 | 4.16061E−15 | −0.43952655 | CAMTA1 |
| chr1 | 7574506 | 7574692 | 6.9467E−11 | 0.403683438 | CAMTA1 |
| chr1 | 8876618 | 8876723 | 1.12059E−13 | −0.528876038 | ENO1 |
| chr1 | 9426714 | 9427020 | 6.9467E−11 | −0.369941498 | LINC02606 |
| chr1 | 9522246 | 9522460 | 9.2458E−17 | −0.427697004 | SLC25A33 |
| chr1 | 10032019 | 10032210 | 1.3454E−11 | −0.456640893 | UBE4B |
| chr1 | 10473426 | 10473579 | 1.78134E−08 | −0.407496023 | PEX14 |
| chr1 | 10779238 | 10779518 | 8.96843E−15 | −0.502352221 | CASZ1 |
| chr1 | 10976445 | 10976641 | 8.96843E−15 | −0.447737475 | C1orf127 |
| chr1 | 11501400 | 11501552 | 5.32836E−13 | 0.424868849 | DISP3 |
| chr1 | 12053417 | 12053802 | 4.16061E−15 | 0.427618362 | TNFRSF8 |
| chr1 | 15123853 | 15124087 | 3.07638E−10 | −0.379380274 | TMEM51-AS1 |
| chr1 | 15214700 | 15214853 | 3.4753E−07 | 0.377535634 | TMEM51 |
| chr1 | 15427126 | 15427236 | 1.06021E−06 | 0.359085511 | EFHD2 |
| chr1 | 16143466 | 16144191 | 3.72444E−09 | −0.448356664 | EPHA2 |
| chr1 | 16500058 | 16500178 | 1.84916E−16 | −0.412153329 | CROCCP3 |
| chr1 | 16623926 | 16624445 | 1.46991E−07 | −0.3732013 | CROCCP2 |
| chr1 | 18927284 | 18927586 | 8.59398E−13 | −0.402178467 | IFFO2 |
| chr1 | 19984613 | 19984776 | 1.70548E−12 | −0.397963817 | PLA2G2A |
| chr1 | 22114498 | 22114674 | 7.87923E−07 | 0.282178617 | WNT4 |
| chr1 | 22239673 | 22239875 | 2.12644E−12 | −0.39822544 | MIR4418 |
| chr1 | 22809031 | 22809263 | 3.44869E−14 | 0.489197751 | EPHB2 |
| chr1 | 25771760 | 25771901 | 1.70548E−12 | −0.425344987 | MAN1C1 |
| chr1 | 30910068 | 30910263 | 4.16061E−15 | −0.512112845 | SDC3 |
| chr1 | 36258000 | 36258135 | 1.7567E−15 | −0.46830557 | THRAP3 |
| chr1 | 37475596 | 37475796 | 6.65919E−10 | −0.475739055 | ZC3H12A |
| chr1 | 37932460 | 37932663 | 8.59398E−13 | −0.394681538 | INPP5B |
| chr1 | 39467087 | 39467223 | 8.25256E−11 | 0.445877644 | MACF1 |
| chr1 | 39498363 | 39498671 | 1.36237E−12 | 0.44628821 | BMP8A |
| chr1 | 39606707 | 39606865 | 4.16801E−13 | −0.422167602 | HEYL |
| chr1 | 39819646 | 39819737 | 2.46079E−09 | −0.38243779 | TRIT1 |
| chr1 | 40312574 | 40313052 | 4.89446E−11 | −0.427838835 | COL9A2 |
| chr1 | 41501474 | 41501929 | 1.12059E−13 | −0.473733708 | HIVEP3 |
| chr1 | 45622296 | 45623241 | 9.2458E−17 | 0.50300194 | CCDC17 |
| chr1 | 46305035 | 46305173 | 1.17715E−07 | −0.409501155 | UQCRH |
| chr1 | 46331839 | 46332671 | 3.07638E−10 | −0.435867221 | NSUN4 |
| chr1 | 53526868 | 53526957 | 3.24158E−13 | 0.456262382 | GLIS1 |
| chr1 | 54781125 | 54781356 | 1.7567E−15 | 0.451133877 | TTC22 |
| chr1 | 58814094 | 58814794 | 1.70548E−12 | −0.417648463 | LINC01135 |
| chr1 | 61447566 | 61447883 | 1.36869E−10 | −0.419775302 | NFIA |
| chr1 | 62194712 | 62196015 | 2.51486E−14 | 0.343955544 | L1TD1 |
| chr1 | 67754073 | 67754277 | 1.70548E−12 | −0.473903165 | GNG12 |
| chr1 | 79006722 | 79006852 | 2.50931E−13 | 0.512986917 | ADGRL4 |
| chr1 | 95955839 | 95956002 | 5.72228E−10 | −0.410470206 | LINC02790 |
| chr1 | 96589184 | 96589321 | 6.10121E−12 | 0.47712808 | PTBP2 |
| chr1 | 105294151 | 105294358 | 1.9296E−13 | −0.438090631 | LINC01676 |
| chr1 | 116494438 | 116494622 | 1.36237E−12 | −0.493651277 | LINC01762 |
| chr1 | 118993119 | 118993137 | 3.45806E−08 | 0.394924392 | TBX15 |
| chr1 | 121010837 | 121010898 | 2.77374E−15 | 0.44280318 | PDE4DIPP4 |
| chr1 | 121116929 | 121117076 | 3.44869E−14 | 0.53207203 | H2BP1 |
| chr1 | 145168928 | 145169080 | 4.16061E−15 | 0.51914024 | LINC01145 |
| chr1 | 145872129 | 145872347 | 2.01703E−08 | 0.382429257 | ANKRD35 |
| chr1 | 147077001 | 147077607 | 4.16801E−13 | 0.428221897 | NUDT4B |
| chr1 | 147224669 | 147224807 | 1.61457E−10 | −0.446335508 | CHD1L |
| chr1 | 147423320 | 147423664 | 2.50931E−13 | −0.516501001 | NUDT4B |
| chr1 | 147587078 | 147587401 | 3.44869E−14 | −0.485407962 | BCL9 |
| chr1 | 147618877 | 147618982 | 2.07662E−06 | 0.299544812 | BCL9 |
| chr1 | 148048532 | 148049023 | 4.97202E−12 | 0.391105099 | NUDT4B |
| chr1 | 148310102 | 148310320 | 8.59398E−13 | 0.459218986 | NUDT4B |
| chr1 | 148361472 | 148361564 | 1.2884E−06 | 0.380276069 | NUDT4B |
| chr1 | 149699079 | 149699238 | 5.22319E−12 | 0.441707368 | LOC644634 |
| chr1 | 150158642 | 150159507 | 1.12059E−13 | 0.466845165 | PLEKHO1 |
| chr1 | 151046991 | 151047101 | 3.27265E−12 | −0.436925695 | BNIPL |
| chr1 | 151194903 | 151195676 | 1.28517E−14 | −0.415393904 | VPS72 |
| chr1 | 151837962 | 151839144 | 1.46991E−07 | 0.352119916 | C2CD4D |
| chr1 | 153987276 | 153987441 | 4.16801E−13 | −0.433090696 | RAB13 |
| chr1 | 154713063 | 154713288 | 4.69687E−14 | −0.481026685 | KCNN3 |
| chr1 | 154970114 | 154970487 | 8.29042E−09 | 0.405243294 | SHC1 |
| chr1 | 155188692 | 155188745 | 6.65919E−10 | −0.417114072 | MUC1 |
| chr1 | 155191975 | 155192144 | 9.78564E−11 | −0.464978048 | MUC1 |
| chr1 | 155192834 | 155193008 | 1.84916E−16 | −0.412019547 | MUC1 |
| chr1 | 156697051 | 156697200 | 1.15832E−10 | −0.394813423 | CRABP2 |
| chr1 | 158069011 | 158069418 | 5.25922E−07 | 0.267327585 | KIRREL1 |
| chr1 | 160263205 | 160263511 | 1.22038E−08 | −0.381809838 | PEX19 |
| chr1 | 161022562 | 161022783 | 1.47656E−13 | −0.42673455 | F11R |
| chr1 | 161079645 | 161079771 | 1.7567E−15 | 0.452356979 | NECTIN4 |
| chr1 | 161133943 | 161134171 | 3.24158E−13 | −0.416265537 | DEDD |
| chr1 | 164575967 | 164576930 | 9.2458E−17 | 0.542201599 | PBX1 |
| chr1 | 164712328 | 164712685 | 6.78457E−13 | 0.550956718 | PBX1 |
| chr1 | 167539976 | 167540110 | 2.13847E−09 | −0.364723862 | CREG1 |
| chr1 | 168085486 | 168085885 | 2.51486E−14 | 0.528811941 | GPR161 |
| chr1 | 172431755 | 172432042 | 8.59398E−13 | −0.406337321 | C1orf105 |
| chr1 | 172851303 | 172851516 | 4.69687E−14 | 0.414897824 | FASLG |
| chr1 | 176904140 | 176904405 | 2.85148E−11 | 0.411824439 | ASTN1 |
| chr1 | 178486818 | 178487169 | 2.37133E−11 | 0.429184915 | RASAL2 |
| chr1 | 183160282 | 183160478 | 1.7567E−15 | −0.516721422 | LAMC1 |
| chr1 | 184621554 | 184621841 | 5.83673E−11 | 0.448024902 | C1orf21 |
| chr1 | 197914008 | 197914099 | 0.0249542 | 0.26709962 | LHX9 |
| chr1 | 200058522 | 200058605 | 3.4212E−11 | −0.445254682 | NR5A2 |
| chr1 | 200873627 | 200874246 | 5.32836E−13 | 0.39455633 | GPR25 |
| chr1 | 200916308 | 200916444 | 9.2458E−17 | 0.439635995 | INAVA |
| chr1 | 201283923 | 201284304 | 1.28517E−14 | 0.439698797 | PKP1 |
| chr1 | 202010404 | 202010673 | 3.44869E−14 | −0.527603896 | ELF3 |
| chr1 | 202012703 | 202013334 | 8.45991E−14 | −0.445007722 | ELF3 |
| chr1 | 202168089 | 202168439 | 2.62456E−10 | 0.390374813 | PTPRVP |
| chr1 | 202198980 | 202199667 | 8.45991E−14 | −0.419228869 | LGR6 |
| chr1 | 203631851 | 203631994 | 1.10836E−11 | −0.405438357 | ATP2B4 |
| chr1 | 204089131 | 204089717 | 6.78457E−13 | 0.340833805 | SOX13 |
| chr1 | 204698405 | 204698600 | 1.62897E−11 | −0.427599099 | LRRN2 |
| chr1 | 204752219 | 204752391 | 3.27265E−12 | −0.421583013 | LRRN2 |
| chr1 | 205304315 | 205304496 | 3.4212E−11 | −0.408309929 | NUAK2 |
| chr1 | 205320454 | 205320560 | 9.10841E−12 | −0.52874841 | NUAK2 |
| chr1 | 205360321 | 205360425 | 1.05228E−07 | 0.354571032 | KLHDC8A |
| chr1 | 206052778 | 206053225 | 6.10121E−12 | −0.550308923 | RHEX |
| chr1 | 206063625 | 206063990 | 5.32836E−13 | −0.424866584 | RHEX |
| chr1 | 209231500 | 209232127 | 6.47206E−16 | 0.399928027 | LINC01696 |
| chr1 | 209652155 | 209652442 | 2.37133E−11 | −0.455814915 | LAMB3 |
| chr1 | 210252733 | 210253070 | 1.15832E−10 | −0.429760563 | SERTAD4 |
| chr1 | 212514766 | 212515098 | 6.32413E−14 | 0.455473162 | LINC01740 |
| chr1 | 217631904 | 217632039 | 3.24158E−13 | −0.431957127 | SPATA17 |
| chr1 | 220882301 | 220882501 | 3.44869E−14 | 0.44833908 | MTARC1 |
| chr1 | 220884176 | 220885050 | 2.91289E−08 | 0.340130338 | MTARC1 |
| chr1 | 220894741 | 220894934 | 4.97202E−12 | 0.422667632 | HLX |
| chr1 | 223993817 | 223994333 | 2.12644E−12 | −0.420038067 | SEPTIN7P13 |
| chr1 | 224175709 | 224175823 | 9.43988E−09 | 0.429153876 | DEGS1 |
| chr1 | 225887747 | 225888130 | 3.69832E−16 | 0.444494544 | LEFTY1 |
| chr1 | 226123019 | 226123152 | 2.50931E−13 | −0.468452357 | ACBD3 |
| chr1 | 226132791 | 226133168 | 3.24158E−13 | −0.410256286 | ACBD3 |
| chr1 | 226637681 | 226638399 | 8.45991E−14 | 0.449781805 | ITPKB |
| chr1 | 227560815 | 227561535 | 9.2458E−17 | 0.419087092 | ZNF678 |
| chr1 | 228013420 | 228013656 | 5.32836E−13 | −0.45537014 | WNT3A |
| chr1 | 228212132 | 228212875 | 1.70548E−12 | 0.40109331 | OBSCN |
| chr1 | 228371115 | 228372112 | 3.24158E−13 | −0.452087904 | OBSCN |
| chr1 | 228374742 | 228375359 | 4.97202E−12 | 0.419306811 | OBSCN |
| chr1 | 229138633 | 229138937 | 2.13847E−09 | −0.376132928 | LOC105373159 |
| chr1 | 229570086 | 229570481 | 6.9467E−11 | −0.427062205 | ABCB10 |
| chr1 | 230130444 | 230130658 | 1.15832E−10 | −0.415794283 | GALNT2 |
| chr1 | 231340083 | 231340224 | 1.5621E−06 | −0.401448688 | SPRTN |
| chr1 | 233329181 | 233329579 | 3.4212E−11 | −0.468403043 | MAP3K21 |
| chr1 | 234309474 | 234309666 | 3.69832E−16 | −0.429015073 | SLC35F3 |
| chr1 | 234531446 | 234531565 | 1.04116E−09 | 0.397040812 | LINC01354 |
| chr1 | 235048093 | 235048526 | 8.45991E−14 | 0.477788372 | TOMM20 |
| chr1 | 235073527 | 235074121 | 1.70548E−12 | −0.478286199 | TOMM20 |
| chr1 | 242662344 | 242662554 | 3.24158E−13 | −0.437574546 | PLD5 |
| chr1 | 242786322 | 242786529 | 8.45991E−14 | −0.431576947 | PLD5 |
| chr1 | 242910272 | 242910484 | 5.32836E−13 | −0.418537207 | LINC01347 |
| chr1 | 247347909 | 247348262 | 3.69832E−16 | 0.525901554 | ZNF496 |
| chr2 | 443317 | 443522 | 3.6005E−10 | −0.389679213 | LINC01874 |
| chr2 | 3585300 | 3585818 | 2.77374E−15 | 0.519514875 | RPS7 |
| chr2 | 3594612 | 3595185 | 6.32413E−14 | 0.448511966 | COLEC11 |
| chr2 | 3844337 | 3844456 | 2.12644E−12 | −0.475999799 | DCDC2C |
| chr2 | 7032099 | 7032270 | 4.04042E−12 | −0.444718344 | RNF144A |
| chr2 | 8457714 | 8458014 | 2.77374E−15 | −0.433609932 | LINC01814 |
| chr2 | 8573453 | 8574055 | 3.24158E−13 | −0.411192032 | LINC01814 |
| chr2 | 8575481 | 8575549 | 7.46442E−12 | 0.406292139 | LINC01814 |
| chr2 | 9386921 | 9387139 | 4.26687E−09 | −0.481196459 | ASAP2 |
| chr2 | 10427581 | 10427718 | 0.000480394 | 0.248965409 | HPCAL1 |
| chr2 | 10757220 | 10757563 | 6.19469E−15 | −0.414439246 | ATP6V1C2 |
| chr2 | 15820043 | 15820424 | 7.27381E−09 | −0.396765867 | LINC01804 |
| chr2 | 16226864 | 16227116 | 1.78134E−08 | −0.322795026 | GACAT3 |
| chr2 | 16652592 | 16652775 | 2.38077E−10 | 0.539454724 | CYRIA |
| chr2 | 20348700 | 20348822 | 6.9467E−11 | −0.438506814 | PUM2 |
| chr2 | 20640909 | 20641063 | 6.19469E−15 | 0.400430091 | HS1BP3 |
| chr2 | 23502068 | 23502414 | 1.46991E−07 | −0.304797142 | KLHL29 |
| chr2 | 23554375 | 23554522 | 3.4212E−11 | 0.426247257 | KLHL29 |
| chr2 | 24861633 | 24861827 | 2.77374E−15 | 0.429512822 | ADCY3 |
| chr2 | 25215998 | 25216503 | 0.292995805 | 0.195574377 | LINC01381 |
| chr2 | 26302787 | 26303214 | 1.08444E−12 | −0.402267893 | ADGRF3 |
| chr2 | 26946967 | 26947115 | 6.32413E−14 | −0.426011501 | DPYSL5 |
| chr2 | 27078432 | 27079217 | 1.08444E−12 | 0.436545661 | EMILIN1 |
| chr2 | 27079648 | 27080286 | 1.12059E−13 | 0.499788296 | EMILIN1 |
| chr2 | 28342702 | 28342881 | 2.51486E−14 | 0.484195074 | BABAM2 |
| chr2 | 28436560 | 28437143 | 4.09634E−11 | −0.40630912 | FOSL2 |
| chr2 | 34753189 | 34753370 | 1.08444E−12 | −0.521984506 | LINC01320 |
| chr2 | 38073718 | 38075233 | 6.78457E−13 | 0.409813375 | CYP1B1-AS1 |
| chr2 | 38239521 | 38239839 | 5.83673E−11 | −0.481526176 | CYP1B1-AS1 |
| chr2 | 38323997 | 38324203 | 1.15551E−07 | −0.379695687 | ATL2 |
| chr2 | 39439132 | 39439338 | 2.38506E−10 | 0.50165796 | MAP4K3-DT |
| chr2 | 43169786 | 43170004 | 1.9296E−13 | −0.484658765 | LINC02580 |
| chr2 | 43187020 | 43187201 | 4.89446E−11 | 0.364339282 | ZFP36L2 |
| chr2 | 47041207 | 47041419 | 6.78457E−13 | 0.463820389 | TTC7A |
| chr2 | 47041233 | 47041419 | 6.78457E−13 | 0.45972996 | TTC7A |
| chr2 | 47370357 | 47370665 | 9.2458E−17 | −0.556495653 | EPCAM |
| chr2 | 49933246 | 49933382 | 1.80293E−14 | −0.438523296 | NRXN1 |
| chr2 | 54937001 | 54937226 | 1.70548E−12 | −0.40827817 | EML6 |
| chr2 | 56020667 | 56020803 | 6.19469E−15 | −0.473815647 | MIR217HG |
| chr2 | 57260421 | 57260591 | 6.19469E−15 | −0.424061124 | VRK2 |
| chr2 | 57297779 | 57297981 | 4.16061E−15 | −0.416080865 | VRK2 |
| chr2 | 61793714 | 61793789 | 2.57927E−08 | 0.395588461 | FAM161A |
| chr2 | 62292985 | 62293534 | 2.85148E−11 | −0.467490919 | B3GNT2 |
| chr2 | 64857830 | 64858048 | 2.67355E−09 | 0.447607037 | LINC01800 |
| chr2 | 70896836 | 70896943 | 2.51486E−14 | 0.460249881 | VAX2 |
| chr2 | 71050156 | 71050722 | 2.51486E−14 | −0.39457104 | NAGK |
| chr2 | 71128690 | 71128846 | 8.59398E−13 | −0.424477519 | MCEE |
| chr2 | 71160866 | 71161022 | 4.97202E−12 | −0.548629633 | MPHOSPH10 |
| chr2 | 71160866 | 71161070 | 1.62897E−11 | −0.544478481 | MPHOSPH10 |
| chr2 | 71700229 | 71700603 | 2.51486E−14 | −0.420743625 | DYSF |
| chr2 | 74177110 | 74177278 | 3.07638E−10 | −0.455435456 | MOB1A |
| chr2 | 74177182 | 74177278 | 3.07638E−10 | −0.455200254 | MOB1A |
| chr2 | 85584217 | 85584791 | 2.12644E−12 | 0.397142724 | VAMP5 |
| chr2 | 85765665 | 85765761 | 1.9296E−13 | −0.511228615 | ATOH8 |
| chr2 | 85773151 | 85773401 | 1.84916E−16 | 0.495941113 | ATOH8 |
| chr2 | 87409813 | 87410169 | 1.12059E−13 | −0.388747079 | CYTOR |
| chr2 | 88284078 | 88284507 | 1.84916E−16 | 0,48405646 | THNSL2 |
| chr2 | 94735635 | 94735755 | 2.12644E−12 | 0.366668828 | ANKRD20A8P |
| chr2 | 94807950 | 94808071 | 1.1095E−15 | −0.443694635 | ANKRD20A8P |
| chr2 | 94923119 | 94923724 | 1.08444E−12 | −0.402329479 | LOC442028 |
| chr2 | 95274688 | 95274880 | 8.45991E−14 | −0.401491638 | PROM2 |
| chr2 | 95649305 | 95649453 | 1.60932E−09 | 0.430527923 | TRIM43 |
| chr2 | 96109129 | 96109306 | 4.16061E−15 | 0.469667004 | ADRA2B |
| chr2 | 96500794 | 96500855 | 1.53101E−09 | 0.423848537 | NEURL3 |
| chr2 | 96761568 | 96761811 | 6.3755E−09 | −0.34764551 | CNNM4 |
| chr2 | 98822469 | 98823367 | 9.2458E−17 | 0.509271351 | CRACDL |
| chr2 | 99182276 | 99182471 | 9.43988E−09 | −0.411582346 | MRPL30 |
| chr2 | 100196989 | 100197241 | 1.84916E−16 | 0.453198864 | AFF3 |
| chr2 | 101630344 | 101630736 | 1.62897E−11 | −0.476562115 | MAP4K4 |
| chr2 | 102051010 | 102051390 | 1.1095E−15 | 0.483840218 | IL1R1 |
| chr2 | 106066853 | 106067059 | 1.08444E−12 | −0.409419505 | ECRG4 |
| chr2 | 108579819 | 108580189 | 4.16061E−15 | 0.397759389 | LIMS1 |
| chr2 | 110732047 | 110732159 | 2.62456E−10 | −0.391117406 | ACOXL |
| chr2 | 113712408 | 113712611 | 1.08444E−12 | −0.41395646 | SLC35F5 |
| chr2 | 119678099 | 119678757 | 4.09634E−11 | −0.388975813 | TMEM177 |
| chr2 | 120399028 | 120399505 | 1.36237E−12 | −0.427938065 | INHBB |
| chr2 | 120820369 | 120820591 | 2.91289E−08 | 0.34522902 | GLI2 |
| chr2 | 120868111 | 120868264 | 1.80293E−14 | 0.454420182 | GLI2 |
| chr2 | 127073071 | 127073161 | 9.2458E−17 | 0.459540489 | BIN1 |
| chr2 | 128681298 | 128681453 | 3.6005E−10 | −0.419369826 | HS6ST1 |
| chr2 | 131126450 | 131126697 | 0.000125823 | 0.206121104 | PLEKHB2 |
| chr2 | 131178729 | 131179016 | 8.45991E−14 | 0.437184109 | PLEKHB2 |
| chr2 | 132256329 | 132257112 | 1.7567E−15 | 0.400242226 | ANKRD30BL |
| chr2 | 154699414 | 154699557 | 6.78457E−13 | 0.377328311 | KCNJ3 |
| chr2 | 156322837 | 156323001 | 1.96776E−11 | 0.44733052 | NR4A2 |
| chr2 | 158867636 | 158867963 | 1.78134E−08 | −0.372250427 | DAPL1 |
| chr2 | 160205431 | 160205882 | 4.09634E−11 | −0.408481844 | ITGB6 |
| chr2 | 160648139 | 160648684 | 1.1095E−15 | −0.47671485 | RBMS1 |
| chr2 | 169716708 | 169716983 | 1.90143E−10 | 0.433731501 | PHOSPHO2-KLHL23 |
| chr2 | 170716558 | 170716760 | 6.3755E−09 | 0.401933344 | SP5 |
| chr2 | 172465626 | 172465697 | 4.17463E−08 | 0.442434587 | ITGA6 |
| chr2 | 176099439 | 176099452 | 7.3195E−05 | 0.304226816 | HOXD12 |
| chr2 | 176158199 | 176158460 | 4.16061E−15 | −0.440020537 | HOXD3 |
| chr2 | 185738818 | 185739210 | 2.13847E−09 | 0.339601427 | FSIP2 |
| chr2 | 197501486 | 197501592 | 4.89446E−11 | −0.411160152 | HSPE1 |
| chr2 | 199464782 | 199464859 | 1.1286E−05 | 0.231295601 | SATB2 |
| chr2 | 204561755 | 204561893 | 2.50931E−13 | −0.443826358 | PARD3B |
| chr2 | 207811209 | 207811494 | 4.89446E−11 | −0.433474426 | PLEKHM3 |
| chr2 | 208124334 | 208124601 | 2.85148E−11 | 0.454877134 | CRYGD |
| chr2 | 208529926 | 208530492 | 3.07638E−10 | −0.365858492 | PTH2R |
| chr2 | 217816451 | 217816839 | 8.96843E−15 | 0.509277012 | TNS1 |
| chr2 | 218849911 | 218850119 | 3.44869E−14 | −0.387348694 | WNT6 |
| chr2 | 218871524 | 218871666 | 3.07638E−10 | 0.348069179 | WNT6 |
| chr2 | 222297051 | 222297097 | 1.03594E−08 | 0.33459198 | PAX3 |
| chr2 | 222788012 | 222788148 | 7.46442E−12 | −0.49508184 | ACSL3 |
| chr2 | 223054314 | 223054500 | 4.16061E−15 | 0.500347199 | KCNE4 |
| chr2 | 226795404 | 226796386 | 1.38562E−08 | 0.384560492 | IRS1 |
| chr2 | 230990569 | 230990629 | 4.16801E−13 | 0.423067693 | SPATA3-AS1 |
| chr2 | 231680107 | 231680704 | 8.25256E−11 | −0.422373585 | PTMA |
| chr2 | 232381343 | 232381894 | 3.44869E−14 | 0.41190614 | ALPP |
| chr2 | 232386550 | 232388669 | 9.2458E−17 | 0.51380476 | ECEL1P2 |
| chr2 | 232419233 | 232420023 | 1.36869E−10 | 0.386018227 | ALPG |
| chr2 | 232606745 | 232606822 | 1.1095E−15 | 0.404303893 | EFHD1 |
| chr2 | 233084594 | 233084712 | 3.6005E−10 | −0.427238935 | INPP5D |
| chr2 | 233388089 | 233388198 | 3.27265E−12 | 0.495475788 | DGKD |
| chr2 | 234809102 | 234809474 | 1.36237E−12 | −0.396064435 | LINC01173 |
| chr2 | 234978235 | 234978550 | 3.4212E−11 | −0.472060341 | SH3BP4 |
| chr2 | 234978443 | 234978550 | 4.97202E−12 | −0.490714728 | SH3BP4 |
| chr2 | 236550855 | 236551156 | 1.70548E−12 | −0.452489908 | ACKR3 |
| chr2 | 236633897 | 236634327 | 4.91037E−10 | −0.446266447 | ACKR3 |
| chr | 237278427 | 237278523 | 3.6005E−10 | 0.397237187 | COL6A3 |
| chr2 | 237600452 | 237600680 | 3.69832E−16 | 0.473092012 | RAB17 |
| chr2 | 238573973 | 238574158 | 6.3755E−09 | −0.413125558 | LINC01107 |
| chr2 | 239308241 | 239309189 | 3.44869E−14 | 0.490171029 | HDAC4 |
| chr2 | 239312803 | 239313116 | 6.19469E−15 | 0.452355283 | HDAC4 |
| chr2 | 239465247 | 239465373 | 4.20778E−10 | −0.407825018 | HDAC4-AS1 |
| chr2 | 239736989 | 239737067 | 2.50931E−13 | −0.380248694 | LOC150935 |
| chr2 | 240322787 | 240323088 | 2.12644E−12 | −0.41381055 | GPC1 |
| chr2 | 240520225 | 240520493 | 9.2458E−17 | 0.566619173 | ANKMY1 |
| chr2 | 241065475 | 241065660 | 6.74429E−06 | 0.344700068 | SNED1 |
| chr2 | 241123965 | 241124044 | 1.22038E−08 | −0.424082849 | PASK |
| chr2 | 241700749 | 241700787 | 9.2458E−17 | −0.454824269 | ING5 |
| chr3 | 8996698 | 8996917 | 6.47206E−16 | 0.496828402 | SRGAP3 |
| chr3 | 10552125 | 10552167 | 9.58194E−11 | −0.528327479 | ATP2B2 |
| chr3 | 12441098 | 12441183 | 2.82847E−09 | 0.404248273 | PPARG |
| chr3 | 12924610 | 12924782 | 1.04116E−09 | −0.388042043 | IQSEC1 |
| chr3 | 13484397 | 13484687 | 8.25256E−11 | −0.395614165 | HDAC11 |
| chr3 | 20029245 | 20029704 | 1.62897E−11 | −0.423002042 | KAT2B |
| chr3 | 20049021 | 20049393 | 2.64282E−12 | −0.423782177 | KAT2B |
| chr3 | 23743157 | 23743586 | 2.81406E−07 | −0.3013175 | UBE2E1-AS1 |
| chr3 | 46464618 | 46464742 | 4.91037E−10 | 0.390659135 | LTF |
| chr3 | 47703956 | 47704094 | 2.37133E−11 | −0.38927247 | SMARCC1 |
| chr3 | 52077188 | 52077663 | 6.47206E−16 | 0.509775376 | POC1A |
| chr3 | 54600645 | 54600837 | 3.07638E−10 | 0.409791473 | CACNA2D3 |
| chr3 | 64438933 | 64439074 | 1.84916E−16 | 0.523065334 | PRICKLE2 |
| chr3 | 69198991 | 69199197 | 2.51486E−14 | −0.535107789 | FRMD4B |
| chr3 | 75361762 | 75362322 | 1.80293E−14 | −0.544766447 | FAM86DP |
| chr3 | 75604385 | 75604506 | 6.19469E−15 | −0.441050049 | MIR1324 |
| chr3 | 98770405 | 98770530 | 2.13847E−09 | −0.471771007 | ST3GAL6 |
| chr3 | 101511979 | 101512116 | 3.44869E−14 | −0.451347248 | SENP7 |
| chr3 | 112332319 | 112332449 | 2.46139E−09 | −0.563638894 | CD200 |
| chr3 | 116535725 | 116535862 | 4.69687E−14 | 0.444558962 | LSAMP |
| chr3 | 122005270 | 122005333 | 2.27256E−07 | −0.405718644 | ILDR1 |
| chr3 | 122565233 | 122565369 | 2.62456E−10 | −0.468377614 | DTX3L |
| chr3 | 123330835 | 123331138 | 2.62456E−10 | 0.442442715 | ADCY5 |
| chr3 | 123412924 | 123413071 | 4.16801E−13 | 0.510325993 | ADCY5 |
| chr3 | 123698874 | 123699750 | 4.69687E−14 | 0.45516923 | MYLK |
| chr3 | 126474107 | 126474226 | 5.27153E−10 | 0.449449883 | ZXDC |
| chr3 | 126541659 | 126542603 | 4.69687E−14 | 0.451961167 | CHST13 |
| chr3 | 127043118 | 127043241 | 3.27265E−12 | −0.419284351 | PLXNA1 |
| chr3 | 127304125 | 127304594 | 8.96843E−15 | −0.419843335 | PRR20G |
| chr3 | 127566913 | 127567261 | 3.24158E−13 | −0.431651649 | TPRA1 |
| chr3 | 127750332 | 127750618 | 1.85622E−09 | −0.427467053 | MGLL |
| chr3 | 128282298 | 128282916 | 2.51486E−14 | 0.492067838 | EEFSEC |
| chr3 | 129561072 | 129561419 | 5.58249E−09 | 0.35801755 | PLXND1 |
| chr3 | 129974512 | 129975112 | 1.15832E−10 | 0.368817901 | TRH |
| chr3 | 130119612 | 130120034 | 3.4212E−11 | 0.414975004 | LINC02021 |
| chr3 | 130144167 | 130144733 | 1.28517E−14 | −0.539986818 | LINC02021 |
| chr3 | 133040306 | 133040497 | 1.70548E−12 | −0.431904936 | TMEM108 |
| chr3 | 140627217 | 140627411 | 4.09634E−11 | −0.428329008 | CLSTN2 |
| chr3 | 141292386 | 141292556 | 1.47656E−13 | 0.503935879 | PXYLP1 |
| chr3 | 141797707 | 141797771 | 9.89883E−09 | 0.509068693 | GRK7 |
| chr3 | 141797707 | 141797771 | 9.89883E−09 | 0.509068693 | GRK7 |
| chr3 | 147384920 | 147385070 | 6.19469E−15 | 0.426665209 | ZIC4 |
| chr3 | 147410326 | 147411186 | 3.44869E−14 | 0.349445005 | ZIC1 |
| chr3 | 149547449 | 149547583 | 1.69867E−12 | 0.596081612 | WWTR1 |
| chr3 | 157542784 | 157542976 | 4.97202E−12 | 0.518816505 | SLC66A1L |
| chr3 | 160450090 | 160450396 | 4.04042E−12 | 0.486575657 | TRIM59-IFT80 |
| chr3 | 165713403 | 165713730 | 1.10836E−11 | −0.3960125 | BCHE |
| chr3 | 170419227 | 170420143 | 2.74694E−06 | 0.302211363 | CLDN11 |
| chr3 | 170585743 | 170585764 | 7.87492E−09 | 0.356841789 | SLC7A14 |
| chr3 | 177245302 | 177245921 | 2.85148E−11 | −0.452843593 | TBL1XR1 |
| chr3 | 181141171 | 181141373 | 3.6005E−10 | −0.412975381 | SOX2-OT |
| chr3 | 184338681 | 184339082 | 3.12829E−07 | 0.429832219 | FAM131A |
| chr3 | 184691896 | 184691959 | 4.20778E−10 | 0.487107435 | MAGEF1 |
| chr3 | 184750851 | 184751021 | 2.37133E−11 | −0.394850964 | LINC02069 |
| chr3 | 185836968 | 185837224 | 1.15832E−10 | −0.433694537 | IGF2BP2 |
| chr3 | 186193681 | 186194566 | 8.25256E−11 | 0.433358198 | DGKG |
| chr3 | 186406591 | 186407181 | 8.96843E−15 | 0.47937331 | LINC02020 |
| chr3 | 186406591 | 186407406 | 1.28517E−14 | 0.4509227 | LINC02020 |
| chr3 | 186435754 | 186436093 | 6.32413E−14 | −0.513505726 | LINC02020 |
| chr3 | 186663558 | 186663684 | 2.77374E−15 | 0.529128932 | HRG |
| chr3 | 193772524 | 193772682 | 9.10841E−12 | −0.434299795 | LINC02038 |
| chr3 | 194322182 | 194322534 | 1.15832E−10 | −0.462436585 | LINC00887 |
| chr3 | 195080439 | 195080901 | 7.46442E−12 | −0.485788433 | XXYLT1 |
| chr3 | 196183520 | 196183673 | 5.83673E−11 | −0.47325741 | ZDHHC19 |
| chr3 | 196660504 | 196660989 | 3.27265E−12 | −0.390293439 | PIGX |
| chr4 | 625651 | 626091 | 6.10121E−12 | −0.38384805 | PDE6B |
| chr4 | 1173416 | 1173582 | 2.77374E−15 | 0.523246195 | SPON2 |
| chr4 | 1710775 | 1710914 | 4.69687E−14 | −0.362610193 | SLBP |
| chr4 | 2430467 | 2430566 | 8.96843E−15 | −0.536552216 | CFAP99 |
| chr4 | 2795504 | 2795683 | 1.61103E−07 | −0.419014307 | SH3BP2 |
| chr4 | 3335463 | 3336046 | 1.08444E−12 | −0.401022077 | RGS12 |
| chr4 | 3463988 | 3464841 | 9.39953E−08 | −0.374091065 | DOK7 |
| chr4 | 4168193 | 4168683 | 4.26687E−09 | −0.380917387 | OTOP1 |
| chr4 | 4759255 | 4759401 | 1.28517E−14 | −0.544102096 | STX18-AS1 |
| chr4 | 5866073 | 5866472 | 1.15832E−10 | −0.41610419 | CRMP1 |
| chr4 | 6128608 | 6128673 | 2.37133E−11 | −0.419324733 | C4orf50 |
| chr4 | 6385943 | 6386207 | 1.70548E−12 | 0.412590083 | PPP2R2C |
| chr4 | 6576546 | 6576888 | 6.66914E−08 | −0.369960488 | MAN2B2 |
| chr4 | 6746604 | 6746738 | 9.78564E−11 | −0.447795256 | BLOC1S4 |
| chr4 | 6767293 | 6767857 | 1.7567E−15 | −0.425252533 | KIAA0232 |
| chr4 | 7050518 | 7050791 | 6.10121E−12 | 0.427139425 | TADA2B |
| chr4 | 7147780 | 7147869 | 6.10121E−12 | −0.470123612 | LINC02447 |
| chr4 | 7263317 | 7263496 | 1.9296E−13 | −0.452523964 | SORCS2 |
| chr4 | 7324447 | 7324559 | 6.32413E−14 | −0.43651471 | SORCS2 |
| chr4 | 7518727 | 7519172 | 1.08444E−12 | −0.468179131 | SORCS2 |
| chr4 | 8865409 | 8865593 | 1.2053E−09 | 0.358152199 | HMX1 |
| chr4 | 8945980 | 8946140 | 9.2458E−17 | −0.491968016 | HMX1 |
| chr4 | 8976739 | 8977198 | 1.84916E−16 | −0.409708347 | HMX1 |
| chr4 | 8992970 | 8993174 | 4.97202E−12 | −0.384041403 | HMX1 |
| chr4 | 9102429 | 9102996 | 1.47656E−13 | −0.504739636 | FAM90A26 |
| chr4 | 9455511 | 9455622 | 1.09584E−09 | −0.416185962 | DEFB131A |
| chr4 | 13524034 | 13524373 | 1.84916E−16 | 0.43098171 | LINC01097 |
| chr4 | 16228381 | 16228638 | 5.58249E−09 | −0.454616624 | TAPT1-AS1 |
| chr4 | 19455379 | 19456110 | 2.51486E−14 | −0.501954609 | SLIT2 |
| chr4 | 19456661 | 19457247 | 2.77374E−15 | −0.527031965 | SLIT2 |
| chr4 | 22475453 | 22475584 | 2.01703E−08 | −0.374119755 | ADGRA3 |
| chr4 | 37584037 | 37584445 | 1.9296E−13 | −0.511576126 | C4orf19 |
| chr4 | 38702373 | 38702722 | 1.84916E−16 | −0.417030806 | KLF3 |
| chr4 | 40283931 | 40284064 | 9.78564E−11 | 0.389204959 | LINC02265 |
| chr4 | 40514805 | 40514967 | 1.29927E−08 | −0.40393831 | RBM47 |
| chr4 | 40630345 | 40630976 | 1.80293E−14 | −0.419617997 | RBM47 |
| chr4 | 40749496 | 40749592 | 3.24158E−13 | −0.40677759 | NSUN7 |
| chr4 | 40908431 | 40908598 | 1.61457E−10 | −0.407713862 | APBB2 |
| chr4 | 43366608 | 43366748 | 3.24158E−13 | −0.402981402 | LINC02383 |
| chr4 | 43537223 | 43537545 | 1.08444E−12 | −0.443807927 | LINC02383 |
| chr4 | 43833481 | 43833684 | 8.45991E−14 | −0.409358008 | LINC02475 |
| chr4 | 48490734 | 48491289 | 9.10841E−12 | 0.366098248 | ZAR1 |
| chr4 | 48944586 | 48944783 | 4.89446E−11 | −0.415224609 | OCIAD2 |
| chr4 | 56975799 | 56975972 | 6.47206E−16 | −0.412620815 | NOA1 |
| chr4 | 60611739 | 60611943 | 9.2458E−17 | −0.427816987 | MIR548AG1 |
| chr4 | 70837796 | 70838013 | 1.70548E−12 | −0.428410942 | GRSF1 |
| chr4 | 99652490 | 99653322 | 1.62897E−11 | −0.379107783 | C4orf54 |
| chr4 | 116980698 | 116980762 | 0.01304035 | −0.143382187 | TRAM1L1 |
| chr4 | 129096891 | 129097066 | 1.9296E−13 | −0.480830889 | C4orf33 |
| chr4 | 139735513 | 139735842 | 1.80293E−14 | 0.432100163 | MAML3 |
| chr4 | 163493744 | 163494083 | 1.07383E−08 | −0.335521912 | TMA16 |
| chr4 | 168877776 | 168878349 | 0.064895828 | 0.153129857 | CBR4 |
| chr4 | 170091199 | 170091289 | 2.01703E−08 | −0.368723321 | AADAT |
| chr4 | 184382425 | 184382622 | 2.70642E−09 | −0.418231768 | LINC02362 |
| chr4 | 190041127 | 190041220 | 1.4261E−13 | 0.477412996 | FRG2 |
| chr5 | 554461 | 554749 | 9.2458E−17 | −0.43754069 | MIR4456 |
| chr5 | 2546803 | 2547247 | 1.1095E−15 | −0.453043931 | LSINCT5 |
| chr5 | 2576979 | 2577846 | 1.28517E−14 | −0.416540278 | LSINCT5 |
| chr5 | 2699624 | 2699976 | 3.69832E−16 | −0.423785735 | LSINCT5 |
| chr5 | 2750643 | 2751696 | 1.36237E−12 | 0.389076584 | IRX2 |
| chr5 | 2755568 | 2755581 | 1.13901E−09 | 0.424054883 | C5orf38 |
| chr5 | 2996324 | 2996956 | 2.62456E−10 | −0.415750827 | LINC01377 |
| chr5 | 3599291 | 3600252 | 2.51486E−14 | 0.350615606 | IRX1 |
| chr5 | 3622946 | 3623094 | 2.77374E−15 | 0.506433069 | IRX1 |
| chr5 | 3740847 | 3741102 | 2.50931E−13 | −0.459678659 | IRX1 |
| chr5 | 4223089 | 4223295 | 2.91216E−10 | −0.456777166 | LINC02114 |
| chr5 | 6349065 | 6349845 | 3.44869E−14 | −0.453273128 | LINC02145 |
| chr5 | 6766826 | 6767036 | 1.47656E−13 | −0.440873272 | LINC02236 |
| chr5 | 10649328 | 10650140 | 1.47656E−13 | 0.435103974 | ANKRD33B |
| chr5 | 31692817 | 31692981 | 2.51486E−14 | 0.472602946 | PDZD2 |
| chr5 | 50607559 | 50607677 | 1.84916E−16 | −0.563958355 | PARP8 |
| chr5 | 55223226 | 55223684 | 1.08444E−12 | 0.403603157 | MCIDAS |
| chr5 | 55903458 | 55903626 | 5.83673E−11 | −0.391129454 | IL31RA |
| chr5 | 63961011 | 63961070 | 1.59534E−10 | 0.424210102 | HTR1A |
| chr5 | 77077330 | 77077861 | 0.000154226 | 0.297391431 | ZBED3 |
| chr5 | 93587523 | 93588676 | 0.000116218 | 0.259326352 | NR2F1 |
| chr5 | 105413608 | 105413873 | 5.83673E−11 | −0.455413621 | RAB9BP1 |
| chr5 | 115963294 | 115963308 | 0.001400282 | 0.192202424 | LVRN |
| chr5 | 132814054 | 132814578 | 2.27256E−07 | 0.30785792 | SOWAHA |
| chr5 | 139224087 | 139224290 | 4.16801E−13 | −0.514525906 | SIL1 |
| chr5 | 139394667 | 139395301 | 7.46442E−12 | 0.48540835 | PROB1 |
| chr5 | 139526124 | 139526571 | 5.83673E−11 | −0.414521984 | UBE2D2 |
| chr5 | 139543568 | 139543675 | 4.20778E−10 | 0.354275215 | UBE2D2 |
| chr5 | 140672398 | 140672545 | 1.15832E−10 | 0.430574779 | WDR55 |
| chr5 | 140672398 | 140672545 | 1.15832E−10 | 0.430574779 | WDR55 |
| chr5 | 140718910 | 140719249 | 9.10841E−12 | −0.442350868 | VTRNA1-2 |
| chr5 | 140834579 | 140834776 | 1.28517E−14 | 0.431029397 | PCDHA1 |
| chr5 | 140841586 | 140841819 | 1.9296E−13 | 0.389103004 | PCDHA1 |
| chr5 | 141364436 | 141364619 | 8.59398E−13 | 0.395844997 | PCDHGA1 |
| chr5 | 141399317 | 141400134 | 1.1095E−15 | 0.409912217 | PCDHGA8 |
| chr5 | 141409927 | 141410045 | 1.7567E−15 | 0.403981894 | PCDHGA8 |
| chr5 | 141430780 | 141432109 | 1.7567E−15 | 0.412648479 | PCDHGA8 |
| chr5 | 141836113 | 141836419 | 7.73912E−10 | 0.371311733 | PCDH1 |
| chr5 | 145558976 | 145559127 | 9.2458E−17 | −0.574287223 | PRELID2 |
| chr5 | 145558976 | 145559127 | 9.2458E−17 | −0.574287223 | PRELID2 |
| chr5 | 149824931 | 149825095 | 9.2458E−17 | 0.547054474 | PPARGC1B |
| chr5 | 150647951 | 150648212 | 1.10836E−11 | 0.453777071 | SYNPO |
| chr5 | 154835728 | 154835877 | 8.45991E−14 | −0.432188031 | FAXDC2 |
| chr5 | 156975702 | 156975897 | 4.69687E−14 | −0.374502585 | TIMD4 |
| chr5 | 157574552 | 157574584 | 3.44869E−14 | 0.411147054 | ADAM19 |
| chr5 | 168288267 | 168288539 | 3.24158E−13 | −0.430441355 | WWC1 |
| chr5 | 168819342 | 168819458 | 4.09634E−11 | −0.470755824 | SLIT3 |
| chr5 | 172570035 | 172570116 | 0.011371463 | −0.136331336 | LINC01944 |
| chr5 | 172872738 | 172873342 | 1.28517E−14 | 0.55573151 | ERGIC1 |
| chr5 | 173303896 | 173304319 | 2.77374E−15 | −0.415890383 | STC2 |
| chr5 | 173304025 | 173304229 | 9.2458E−17 | −0.501114335 | STC2 |
| chr5 | 173989774 | 173989921 | 5.58249E−09 | 0.410779296 | C5orf47 |
| chr5 | 175255443 | 175255646 | 1.36869E−10 | −0.406510082 | DRD1 |
| chr5 | 176544462 | 176544717 | 6.19469E−15 | −0.379985987 | CDHR2 |
| chr5 | 176798758 | 176798933 | 4.73786E−06 | −0.29785887 | UNC5A |
| chr5 | 176823301 | 176823647 | 4.69895E−08 | −0.362133171 | UNC5A |
| chr5 | 177479666 | 177479878 | 2.70642E−09 | 0.487504481 | PDLIM7 |
| chr5 | 178121454 | 178121628 | 2.50931E−13 | 0.455003142 | N4BP3 |
| chr5 | 178129838 | 178130069 | 1.70548E−12 | −0.412741988 | RMND5B |
| chr5 | 178301805 | 178302260 | 2.64282E−12 | −0.508960523 | COL23A1 |
| chr5 | 178428945 | 178429316 | 7.46442E−12 | −0.420485389 | COL23A1 |
| chr5 | 179051862 | 179052019 | 8.59398E−13 | −0.544023364 | ZNF354C |
| chr5 | 179191042 | 179191732 | 3.24158E−13 | −0.406957028 | ADAMTS2 |
| chr5 | 179204613 | 179204810 | 2.64282E−12 | −0.490177135 | ADAMTS2 |
| chr5 | 179275588 | 179275876 | 1.15832E−10 | 0.413198408 | ADAMTS2 |
| chr5 | 179677480 | 179677830 | 4.09634E−11 | −0.481884962 | CBY3 |
| chr5 | 179807852 | 179808288 | 4.04042E−12 | −0.411026752 | SQSTM1 |
| chr5 | 180127370 | 180127854 | 6.47206E−16 | −0.447262115 | RASGEFIC |
| chr5 | 180170222 | 180170340 | 2.07662E−06 | 0.31907529 | RASGEFIC |
| chr5 | 180603622 | 180603761 | 2.12644E−12 | −0.471216754 | FLT4 |
| chr5 | 180607358 | 180607497 | 2.50931E−13 | −0.419065336 | FLT4 |
| chr5 | 180619942 | 180620312 | 5.32836E−13 | −0.489480968 | FLT4 |
| chr5 | 181058942 | 181059892 | 6.10121E−12 | 0.436172185 | BTNL9 |
| chr5 | 181164615 | 181165137 | 2.50931E−13 | 0.411541652 | LINC01962 |
| chr6 | 386902 | 387097 | 1.08444E−12 | −0.45201981 | IRF4 |
| chr6 | 446953 | 447052 | 7.46442E−12 | −0.421073172 | IRF4 |
| chr6 | 1039118 | 1039506 | 6.32413E−14 | −0.418405327 | LINC01622 |
| chr6 | 1523502 | 1523682 | 2.85148E−11 | −0.448513201 | FOXCUT |
| chr6 | 1593986 | 1594645 | 6.19469E−15 | −0.487766844 | FOXCUT |
| chr6 | 2875368 | 2875442 | 2.02784E−09 | −0.348502656 | SERPINB9P1 |
| chr6 | 4078818 | 4079223 | 5.32836E−13 | 0.404762923 | C6orf201 |
| chr6 | 6803537 | 6803861 | 3.44869E−14 | −0.468196981 | LY86 |
| chr6 | 6803628 | 6803861 | 1.80293E−14 | −0.478641686 | LY86 |
| chr6 | 6820747 | 6821000 | 1.62897E−11 | −0.396510962 | LY86 |
| chr6 | 7203986 | 7204195 | 4.04042E−12 | −0.542525827 | RREB1 |
| chr6 | 10113016 | 10113517 | 1.7567E−15 | 0.446306055 | TFAP2A |
| chr6 | 10381360 | 10382070 | 1.2053E−09 | 0.386899277 | TFAP2A |
| chr6 | 10390212 | 10390321 | 6.19469E−15 | 0.508466911 | TFAP2A |
| chr6 | 10390859 | 10391102 | 2.50931E−13 | 0.409550117 | TFAP2A |
| chr6 | 10393061 | 10393265 | 4.69687E−14 | 0.405074052 | TFAP2A |
| chr6 | 10416103 | 10417819 | 1.08444E−12 | 0.416232295 | TFAP2A |
| chr6 | 11215941 | 11216240 | 4.97202E−12 | −0.390298754 | NEDD9 |
| chr6 | 12288171 | 12288525 | 3.69832E−16 | −0.508779559 | EDN1 |
| chr6 | 13273900 | 13273964 | 1.36237E−12 | −0.493093892 | PHACTR1 |
| chr6 | 14622079 | 14622195 | 1.2053E−09 | −0.395294012 | LINC01108 |
| chr6 | 14998064 | 14998202 | 4.97202E−12 | −0.513832622 | JARID2 |
| chr6 | 16337429 | 16337691 | 1.28517E−14 | 0.555407991 | ATXN1 |
| chr6 | 19892181 | 19892374 | 3.27265E−12 | −0.458544603 | ID4 |
| chr6 | 24776258 | 24776438 | 2.62456E−10 | −0.34041506 | GMNN |
| chr6 | 26225039 | 26225345 | 9.2458E−17 | 0.571505586 | H3C6 |
| chr6 | 26743451 | 26743550 | 1.04116E−09 | 0.431182187 | ZNF322 |
| chr6 | 26745488 | 26745521 | 2.37133E−11 | 0.395766793 | ZNF322 |
| chr6 | 27205565 | 27206110 | 2.77374E−15 | 0.433813626 | PRSS16 |
| chr6 | 27260321 | 27260552 | 6.19469E−15 | 0.453849467 | PRSS16 |
| chr6 | 27283730 | 27284089 | 1.28517E−14 | 0.441998871 | POM121L2 |
| chr6 | 27725982 | 27726153 | 8.96843E−15 | −0.400219201 | LINC01012 |
| chr6 | 27815240 | 27815722 | 0.004745562 | 0.25353853 | H2BC14 |
| chr6 | 27864262 | 27864452 | 3.72444E−09 | 0.386467081 | H2AC16 |
| chr6 | 28091066 | 28091196 | 6.47206E−16 | 0.529025019 | ZSCAN12P1 |
| chr6 | 33599126 | 33599825 | 2.64282E−12 | −0.44526926 | LINC00336 |
| chr6 | 34551011 | 34551164 | 4.69895E−08 | 0.377676104 | SPDEF |
| chr6 | 34555450 | 34555985 | 3.6005E−10 | −0.389064858 | SPDEF |
| chr6 | 35017496 | 35017848 | 5.32836E−13 | 0.428338553 | ANKS1A |
| chr6 | 35722467 | 35722668 | 1.70548E−12 | 0.418473592 | FKBP5 |
| chr6 | 37104028 | 37104306 | 2.77374E−15 | −0.419359441 | PIM1 |
| chr6 | 37577502 | 37577705 | 2.37133E−11 | −0.372912331 | MIR4462 |
| chr6 | 41451592 | 41451680 | 1.84916E−16 | 0.467619596 | FOXP4-AS1 |
| chr6 | 42104294 | 42104890 | 1.7567E−15 | 0.487171926 | C6orf132 |
| chr6 | 42137090 | 42137497 | 8.45991E−14 | −0.457451443 | C6orf132 |
| chr6 | 42178087 | 42178479 | 1.84916E−16 | 0.41316415 | GUCA1A |
| chr6 | 43123721 | 43124097 | 1.80293E−14 | 0.461352968 | PTK7 |
| chr6 | 43576920 | 43577094 | 1.47656E−13 | −0.48762734 | POLH |
| chr6 | 51224995 | 51225108 | 3.07638E−10 | −0.351128179 | LOC101927082 |
| chr6 | 53665704 | 53665872 | 9.78564E−11 | 0.409980308 | KLHL31 |
| chr6 | 70282346 | 70282440 | 6.3755E−09 | 0.364579059 | COL9A1 |
| chr6 | 70283081 | 70283405 | 8.98223E−10 | 0.381201852 | COL9A1 |
| chr6 | 73450714 | 73450847 | 9.43988E−09 | −0.432300252 | CGAS |
| chr6 | 73481791 | 73482230 | 1.33479E−05 | −0.30493438 | MTO1 |
| chr6 | 73523124 | 73523403 | 2.62456E−10 | −0.329251559 | EEF1A1 |
| chr6 | 100447583 | 100448246 | 4.97202E−12 | 0.376834532 | SIM1 |
| chr6 | 100469434 | 100469631 | 1.1095E−15 | 0.392424876 | SIM1 |
| chr6 | 104940393 | 104941085 | 1.7567E−15 | 0.416346432 | LIN28B |
| chr6 | 107634342 | 107635271 | 0.005524665 | 0.21418957 | SOBP |
| chr6 | 111260012 | 111260409 | 2.57927E−08 | −0.375844523 | MFSD4B |
| chr6 | 111290004 | 111290366 | 1.90143E−10 | −0.373534702 | REV3L |
| chr6 | 116682252 | 116682302 | 1.1095E−15 | −0.460345293 | KPNA5 |
| chr6 | 117547798 | 117548362 | 8.59398E−13 | 0.430664875 | GOPC |
| chr6 | 136068575 | 136068622 | 3.27265E−12 | −0.462848412 | LOC644135 |
| chr6 | 136922991 | 136923949 | 2.64282E−12 | 0.336482635 | SLC35D3 |
| chr6 | 137775710 | 137776501 | 1.61457E−10 | −0.381277782 | LINC02539 |
| chr6 | 149718966 | 149719041 | 7.48307E−08 | −0.364457843 | LOC645967 |
| chr6 | 156980627 | 156980738 | 8.63351E−09 | −0.496289557 | ARID1B |
| chr6 | 158631829 | 158631936 | 1.70548E−12 | 0.456599639 | TMEM181 |
| chr6 | 159098744 | 159099008 | 1.96776E−11 | −0.423094444 | TAGAP |
| chr6 | 166557049 | 166557387 | 6.19469E−15 | −0.453246646 | RPS6KA2 |
| chr6 | 166588754 | 166588849 | 1.10836E−11 | −0.409153256 | RPS6KA2 |
| chr6 | 167318913 | 167319000 | 6.37133E−06 | −0.377542185 | UNC93A |
| chr6 | 167546763 | 167547166 | 1.5621E−06 | −0.325578973 | LINC02538 |
| chr6 | 168999470 | 168999809 | 8.45991E−14 | −0.413307574 | LOC101929460 |
| chr6 | 169340625 | 169340705 | 3.44869E−14 | −0.402770916 | WDR27 |
| chr6 | 169420855 | 169421059 | 4.88309E−09 | −0.386079636 | WDR27 |
| chr7 | 539421 | 539684 | 1.36869E−10 | −0.431114226 | PRKAR1B |
| chr7 | 666055 | 666282 | 1.70548E−12 | 0.45257055 | PRKAR1B |
| chr7 | 711399 | 711700 | 3.07638E−10 | 0.422422858 | PRKAR1B |
| chr7 | 771320 | 771440 | 4.91037E−10 | 0.438735535 | DNAAF5 |
| chr7 | 781173 | 781665 | 2.64282E−12 | 0.415069409 | DNAAF5 |
| chr7 | 867154 | 867278 | 1.85668E−05 | 0.289675555 | GET4 |
| chr7 | 1455538 | 1455648 | 9.10841E−12 | −0.41360042 | MICALL2 |
| chr7 | 1558935 | 1559403 | 3.4212E−11 | −0.39855703 | TMEM184A |
| chr7 | 1692000 | 1692327 | 5.93912E−08 | −0.34462014 | ELFN1 |
| chr7 | 2168745 | 2169258 | 3.28689E−08 | −0.383274222 | MAD1L1 |
| chr7 | 2460068 | 2460577 | 7.73912E−10 | −0.398770207 | CHST12 |
| chr7 | 2603228 | 2603405 | 2.50373E−06 | −0.311133779 | IQCE |
| chr7 | 2734839 | 2735042 | 6.32413E−14 | 0.431010869 | GNA12 |
| chr7 | 2993626 | 2993775 | 2.01703E−08 | −0.282219911 | CARD11 |
| chr7 | 3239430 | 3239630 | 2.07662E−06 | −0.33643081 | SDK1 |
| chr7 | 4650790 | 4650903 | 6.3755E−09 | 0.387130398 | FOXK1 |
| chr7 | 4879136 | 4879178 | 4.20778E−10 | −0.393366319 | RADIL |
| chr7 | 5248187 | 5248538 | 6.19469E−15 | 0.45385803 | WIPI2 |
| chr7 | 15396275 | 15396619 | 2.85148E−11 | 0.416352437 | AGMO |
| chr7 | 21200909 | 21201110 | 8.38991E−08 | 0.35540434 | LINC01162 |
| chr7 | 23532439 | 23532522 | 4.04042E−12 | −0.415787665 | TRA2A |
| chr7 | 27108112 | 27109061 | 4.69687E−14 | 0.431903308 | HOXA3 |
| chr7 | 29350773 | 29350965 | 4.09634E−11 | −0.38513598 | CHN2 |
| chr7 | 30149075 | 30149456 | 4.89446E−11 | −0.411383042 | MTURN |
| chr7 | 30470296 | 30470499 | 1.36869E−10 | 0.363963062 | NOD1 |
| chr7 | 30988893 | 30988932 | 3.24158E−13 | −0.472982083 | GHRHR |
| chr7 | 31082206 | 31082340 | 1.36237E−12 | −0.454288464 | ADCYAP1R1 |
| chr7 | 32428247 | 32428330 | 1.96776E−11 | 0.403880439 | PDE1C |
| chr7 | 33722397 | 33722642 | 2.37133E−11 | −0.469575576 | BBS9 |
| chr7 | 34487037 | 34487131 | 1.60932E−09 | −0.478486454 | NPSR1-AS1 |
| chr7 | 36313018 | 36313273 | 4.09634E−11 | −0.419355973 | KIAA0895 |
| chr7 | 37211121 | 37211342 | 1.99283E−08 | 0.423917429 | ELMO1 |
| chr7 | 40117204 | 40117409 | 6.10121E−12 | −0.412155423 | MPLKIP |
| chr7 | 43059195 | 43059400 | 6.66914E−08 | −0.38779183 | HECW1 |
| chr7 | 45564672 | 45564785 | 6.32413E−14 | 0.467427969 | ADCY1 |
| chr7 | 46682025 | 46682160 | 1.84916E−16 | 0.427171915 | LOC730338 |
| chr7 | 47969959 | 47970090 | 9.78564E−11 | −0.406781502 | HUS1 |
| chr7 | 54833157 | 54833392 | 4.97202E−12 | −0.529722962 | SEC61G-DT |
| chr7 | 55026547 | 55026650 | 8.98209E−13 | 0.437593357 | EGFR |
| chr7 | 63208346 | 63208397 | 9.59996E−11 | −0.433100292 | ZNF733P |
| chr7 | 66414298 | 66414625 | 6.19469E−15 | −0.495769721 | TPST1 |
| chr7 | 66700233 | 66700488 | 2.51486E−14 | 0.48256725 | RABGEF1 |
| chr7 | 70729112 | 70730043 | 9.2458E−17 | 0.394187099 | AUTS2 |
| chr7 | 72435548 | 72435716 | 3.4212E−11 | 0.397737473 | CALN1 |
| chr7 | 73636131 | 73636272 | 2.50931E−13 | −0.408550563 | MLXIPL |
| chr7 | 74042283 | 74043177 | 4.97202E−12 | 0.407153155 | ELN |
| chr7 | 76251466 | 76251670 | 2.77374E−15 | −0.412309351 | SRRM3 |
| chr7 | 92425101 | 92425334 | 2.62456E−10 | −0.390074569 | TMBIM7P |
| chr7 | 95396483 | 95396726 | 8.74351E−06 | 0.358010916 | PON3 |
| chr7 | 97951642 | 97951767 | 1.08444E−12 | −0.485751784 | ASNS |
| chr7 | 98354383 | 98354863 | 2.51486E−14 | 0.498361762 | BAIAP2L1 |
| chr7 | 98818901 | 98819300 | 4.7463E−07 | −0.419723799 | TMEM130 |
| chr7 | 99274546 | 99274766 | 1.61457E−10 | −0.484875217 | MYH16 |
| chr7 | 99274546 | 99274766 | 1.61457E−10 | −0.484875217 | MYH16 |
| chr7 | 100337984 | 100338352 | 4.16801E−13 | −0.44994391 | STAG3L5P-PVRIG2P- |
| | | | | | PILRB |
| chr7 | 101303385 | 101303635 | 1.3454E−11 | 0.402895896 | LNCPRESS1 |
| chr7 | 101934742 | 101935012 | 2.50931E−13 | 0.46975159 | CUX1 |
| chr7 | 101936437 | 101937119 | 1.08444E−12 | 0.52957723 | CUX1 |
| chr7 | 102300611 | 102301187 | 9.2458E−17 | 0.436539592 | SH2B2 |
| chr7 | 120110224 | 120110387 | 4.16061E−15 | −0.44438753 | KCND2 |
| chr7 | 123390367 | 123390558 | 1.96776E−11 | −0.425009731 | IQUB |
| chr7 | 123390367 | 123390558 | 1.96776E−11 | −0.425009731 | IQUB |
| chr7 | 128270873 | 128271234 | 1.47656E−13 | 0.422665702 | LEP |
| chr7 | 128858911 | 128859458 | 1.9296E−13 | 0.438344161 | FLNC |
| chr7 | 128915606 | 128916487 | 4.16061E−15 | 0.432750145 | KCP |
| chr7 | 129954820 | 129954883 | 1.1479E−09 | −0.506003024 | UBE2H |
| chr7 | 130008522 | 130009216 | 1.1095E−15 | −0.401562529 | ZC3HC1 |
| chr7 | 135208431 | 135208891 | 1.2053E−09 | −0.424882709 | WDR91 |
| chr7 | 140477481 | 140477767 | 1.85622E−09 | −0.307663536 | MKRN1 |
| chr7 | 140485720 | 140485881 | 1.80293E−14 | −0.428431329 | MKRN1 |
| chr7 | 143408073 | 143408683 | 2.77374E−15 | −0.455585902 | EPHA1-AS1 |
| chr7 | 143885165 | 143885496 | 2.23571E−10 | 0.436022902 | TCAF1 |
| chr7 | 146398022 | 146398526 | 1.36237E−12 | −0.426068307 | CNTNAP2 |
| chr7 | 148966330 | 148966548 | 1.80293E−14 | −0.454870226 | RNY4 |
| chr7 | 149820900 | 149821122 | 8.98223E−10 | −0.367026937 | SSPOP |
| chr7 | 151113711 | 151114582 | 1.47656E−13 | −0.434656819 | AGAP3 |
| chr7 | 151115840 | 151115939 | 2.23571E−10 | −0.44618769 | AGAP3 |
| chr7 | 151349883 | 151350091 | 1.7567E−15 | −0.46294304 | NUB1 |
| chr7 | 151712059 | 151712728 | 3.27265E−12 | −0.437679725 | PRKAG2 |
| chr7 | 151932943 | 151933078 | 9.2458E−17 | −0.480940161 | GALNTL5 |
| chr7 | 155233719 | 155233824 | 1.2884E−06 | 0.350593284 | INSIG1 |
| chr7 | 155806075 | 155806720 | 4.16801E−13 | 0.423658485 | SHH |
| chr7 | 155813824 | 155814536 | 5.32836E−13 | 0.386697633 | SHH |
| chr7 | 155951540 | 155951973 | 5.32836E−13 | −0.47461504 | LOC389602 |
| chr7 | 156127578 | 156127802 | 1.39359E−09 | −0.385390315 | LOC389602 |
| chr7 | 157488455 | 157488515 | 8.98223E−10 | −0.461299567 | LOC101927914 |
| chr7 | 157630472 | 157630585 | 1.47656E−13 | −0.479595517 | PTPRN2 |
| chr7 | 158000437 | 158000664 | 2.51486E−14 | −0.433298917 | PTPRN2 |
| chr7 | 158001562 | 158001943 | 1.28517E−14 | −0.438141602 | PTPRN2 |
| chr7 | 158712416 | 158712701 | 4.20778E−10 | −0.44616954 | NCAPG2 |
| chr7 | 158712416 | 158712701 | 4.20778E−10 | −0.44616954 | NCAPG2 |
| chr8 | 11473976 | 11474170 | 4.16801E−13 | −0.404067638 | FAM167A |
| chr8 | 11679480 | 11680747 | 7.73912E−10 | 0.282553135 | GATA4 |
| chr8 | 16456521 | 16456590 | 2.37133E−11 | −0.403304952 | MSR1 |
| chr8 | 17628523 | 17628732 | 1.39359E−09 | −0.431467244 | PDGFRL |
| chr8 | 23071048 | 23071271 | 1.70548E−12 | 0.439806329 | LOC286059 |
| chr8 | 23225642 | 23225781 | 4.91037E−10 | −0.427769271 | LOC389641 |
| chr8 | 23420910 | 23421133 | 8.59398E−13 | −0.37842518 | ENTPD4 |
| chr8 | 34918507 | 34918741 | 6.65919E−10 | −0.415319789 | LINC01288 |
| chr8 | 37782038 | 37782183 | 1.9296E−13 | 0.472132327 | ADGRA2 |
| chr8 | 37787737 | 37788211 | 9.2458E−17 | 0.427581415 | ADGRA2 |
| chr8 | 37898676 | 37898751 | 4.88309E−09 | −0.32313116 | RAB11FIP1 |
| chr8 | 38650812 | 38651296 | 1.7567E−15 | 0.461620462 | RNF5P1 |
| chr8 | 38729158 | 38729425 | 8.45991E−14 | 0.421500742 | TACC1 |
| chr8 | 42297152 | 42297434 | 1.60932E−09 | 0.383760725 | IKBKB |
| chr8 | 42501189 | 42501251 | 7.27381E−09 | 0.383540879 | SLC20A2 |
| chr8 | 48544972 | 48545294 | 1.1095E−15 | −0.448927427 | LOC101929268 |
| chr8 | 48555062 | 48555498 | 7.46442E−12 | 0.482988456 | LOC101929268 |
| chr8 | 51809264 | 51809381 | 2.13847E−09 | 0.431583919 | PXDNL |
| chr8 | 54023048 | 54023246 | 3.69832E−16 | −0.497416888 | TCEA1 |
| chr8 | 54554931 | 54555108 | 8.59398E−13 | −0.503561348 | RP1 |
| chr8 | 54913806 | 54913983 | 6.32413E−14 | −0.42855176 | RP1 |
| chr8 | 57114138 | 57114214 | 6.10121E−12 | −0.408592485 | LINC01606 |
| chr8 | 58146211 | 58146673 | 1.28517E−14 | −0.459274168 | FAM110B |
| chr8 | 66926500 | 66926705 | 5.72228E−10 | −0.435640221 | SNHG6 |
| chr8 | 73370576 | 73371002 | 2.81406E−07 | 0.366160411 | RDH10-AS1 |
| chr8 | 73934215 | 73934647 | 1.96776E−11 | −0.487244978 | ELOC |
| chr8 | 74600350 | 74600668 | 2.12644E−12 | −0.393567486 | MIR2052HG |
| chr8 | 79499927 | 79500150 | 1.7567E−15 | −0.430495806 | STMN2 |
| chr8 | 80201580 | 80201785 | 2.37133E−11 | −0.510269836 | TPD52 |
| chr8 | 86069449 | 86069591 | 1.84916E−16 | 0.411510942 | PSKH2 |
| chr8 | 91448565 | 91448685 | 6.19469E−15 | −0.443906199 | SLC26A7 |
| chr8 | 95026187 | 95026353 | 9.39953E−08 | −0.442304934 | NDUFAF6 |
| chr8 | 97757782 | 97758067 | 2.51486E−14 | −0.558337117 | LAPTM4B |
| chr8 | 98184271 | 98184527 | 4.16061E−15 | 0.531851358 | NIPAL2 |
| chr8 | 98948896 | 98949041 | 0.001134999 | 0.27051607 | OSR2 |
| chr8 | 102627211 | 102627453 | 1.3454E−11 | 0.419498072 | KLF10 |
| chr8 | 102685994 | 102686161 | 6.93983E−08 | 0.430341208 | LOC101927245 |
| chr8 | 111730695 | 111730837 | 4.69687E−14 | −0.408189914 | LINC02237 |
| chr8 | 117944324 | 117944584 | 2.64282E−12 | −0.482068214 | EXT1 |
| chr8 | 120125974 | 120126259 | 6.47206E−16 | 0.45192583 | COL14A1 |
| chr8 | 122887709 | 122887889 | 1.80293E−14 | 0.533424815 | ZHX2 |
| chr8 | 123276514 | 123276648 | 1.47656E−13 | −0.457614342 | ZHX1 |
| chr8 | 123865701 | 123865904 | 1.34026E−05 | −0.574048756 | FER1L6 |
| chr8 | 124827194 | 124827442 | 2.51486E−14 | −0.403927237 | LINC00964 |
| chr8 | 124895274 | 124895397 | 1.47656E−13 | −0.468107488 | LINC00964 |
| chr8 | 124906872 | 124907245 | 7.46442E−12 | −0.466945978 | LINC00964 |
| chr8 | 124906872 | 124907245 | 7.46442E−12 | −0.466945978 | LINC00964 |
| chr8 | 124995553 | 124995624 | 1.15832E−10 | −0.492348002 | SQLE-DT |
| chr8 | 125576208 | 125576444 | 2.62456E−10 | −0.455006105 | TRIB1 |
| chr8 | 126581286 | 126581535 | 3.27265E−12 | −0.541282426 | LRATD2 |
| chr8 | 126833716 | 126833939 | 3.07638E−10 | 0.444036019 | PCAT1 |
| chr8 | 126833716 | 126833939 | 3.07638E−10 | 0.444036019 | PCAT1 |
| chr8 | 127926355 | 127926468 | 1.9296E−13 | 0.49395014 | PVT1 |
| chr8 | 128031197 | 128031434 | 3.21678E−10 | −0.582576033 | PVT1 |
| chr8 | 132674749 | 132674866 | 6.74429E−06 | 0.339406447 | DNAAF11 |
| chr8 | 139601046 | 139601227 | 4.04042E−12 | −0.43700897 | KCNK9 |
| chr8 | 139789185 | 139789244 | 1.46686E−08 | 0.360969557 | TRAPPC9 |
| chr8 | 139918931 | 139919232 | 6.9467E−11 | −0.388723365 | TRAPPC9 |
| chr8 | 140090316 | 140090660 | 1.62897E−11 | −0.436822585 | TRAPPC9 |
| chr8 | 140491193 | 140491389 | 1.96776E−11 | −0.463014117 | CHRAC1 |
| chr8 | 141227609 | 141227959 | 1.12059E−13 | −0.473035876 | SLC45A4 |
| chr8 | 141230856 | 141231013 | 2.64282E−12 | −0.506494196 | SLC45A4 |
| chr8 | 141303157 | 141303293 | 7.73912E−10 | −0.400769903 | SLC45A4 |
| chr8 | 141531345 | 141531402 | 8.59398E−13 | −0.408983882 | MROH5 |
| chr8 | 141605650 | 141605818 | 1.85622E−09 | −0.386433313 | MROH5 |
| chr8 | 141865679 | 141865860 | 8.96843E−15 | −0.421816205 | MIR1302-7 |
| chr8 | 142015982 | 142016288 | 3.28689E−08 | −0.417834469 | MIR4472-1 |
| chr8 | 142205461 | 142205650 | 4.69687E−14 | −0.455690809 | LINC00051 |
| chr8 | 42235290 | 142235413 | 5.58249E−09 | −0.44336842 | TSNARE1 |
| chr8 | 142253018 | 142253241 | 1.08444E−12 | −0.385535043 | TSNARE1 |
| chr8 | 142375332 | 142375593 | 2.50931E−13 | −0.428273079 | TSNARE1 |
| chr8 | 142393790 | 142394409 | 1.15832E−10 | −0.474322903 | TSNARE1 |
| chr8 | 142476620 | 142476921 | 2.50931E−13 | −0.447633044 | ADGRB1 |
| chr8 | 142773177 | 142773524 | 1.3454E−11 | −0.47474337 | LYNX1 |
| chr8 | 143261975 | 143262100 | 3.27265E−12 | −0.472536339 | ZFP41 |
| chr8 | 143877264 | 143877807 | 6.10121E−12 | −0.453170091 | EPPK1 |
| chr8 | 143905987 | 143906197 | 3.44869E−14 | −0.455752376 | PLEC |
| chr8 | 143936232 | 143936760 | 1.2053E−09 | −0.381222699 | PLEC |
| chr8 | 143938737 | 143939004 | 3.4212E−11 | −0.419222803 | PLEC |
| chr8 | 144269308 | 144269437 | 1.10836E−11 | −0.467947747 | BOP1 |
| chr8 | 144472024 | 144472216 | 2.77374E−15 | −0.400591118 | KIFC2 |
| chr8 | 144514993 | 144515236 | 1.47656E−13 | −0.521984046 | RECQL4 |
| chr9 | 18099729 | 18099922 | 4.16061E−15 | −0.43621326 | ADAMTSL1 |
| chr9 | 21967297 | 21967491 | 2.51486E−14 | 0.461246999 | CDKN2A-DT |
| chr9 | 21990048 | 21990100 | 1.84916E−16 | 0.518201978 | MTAP |
| chr9 | 27588506 | 27588688 | 1.9296E−13 | 0.537437371 | C9orf72 |
| chr9 | 29779992 | 29780271 | 1.10836E−11 | −0.415917764 | LINC01242 |
| chr9 | 35036128 | 35036738 | 3.4212E−11 | 0.410942687 | C9orf131 |
| chr9 | 38070076 | 38070205 | 3.4212E−11 | −0.411193063 | SHB |
| chr9 | 38646798 | 38646934 | 3.4212E−11 | −0.474530929 | FAM201A |
| chr9 | 63346325 | 63346606 | 6.78457E−13 | −0.43844503 | LOC286297 |
| chr9 | 69120416 | 69120523 | 8.25256E−11 | −0.458892302 | TJP2 |
| chr9 | 77012161 | 77012259 | 1.71857E−06 | 0.35385357 | FOXB2 |
| chr9 | 77014025 | 77014377 | 2.50931E−13 | 0.451505711 | FOXB2 |
| chr9 | 77016073 | 77016803 | 5.83673E−11 | 0.383850729 | FOXB2 |
| chr9 | 77020766 | 77020997 | 1.36869E−10 | 0.416944962 | FOXB2 |
| chr9 | 77022730 | 77023246 | 3.24158E−13 | 0.442708066 | FOXB2 |
| chr9 | 86959460 | 86959596 | 5.72228E−10 | −0.408193317 | GAS1RR |
| chr9 | 88706237 | 88707016 | 1.36869E−10 | −0.458859034 | MIR4289 |
| chr9 | 89438825 | 89439085 | 8.25256E−11 | −0.417911769 | SEMA4D |
| chr9 | 89639382 | 89639802 | 5.32836E−13 | 0.413030269 | LOC100129066 |
| chr9 | 89664938 | 89665315 | 1.08444E−12 | −0.501429547 | LOC100129066 |
| chr9 | 90227256 | 90227817 | 4.16061E−15 | −0.477239264 | LINC01508 |
| chr9 | 90755565 | 90755671 | 1.17059E−10 | −0.457915862 | SYK |
| chr9 | 94669716 | 94669776 | 3.4212E−11 | −0.414121484 | FBP1 |
| chr9 | 96033889 | 96034198 | 4.28069E−07 | −0.320220029 | ERCC6L2 |
| chr9 | 97854747 | 97854995 | 4.69687E−14 | 0.396980956 | FOXE1 |
| chr9 | 98087579 | 98088067 | 6.78457E−13 | 0.436639828 | TRIM14 |
| chr9 | 107639838 | 107640042 | 1.77972E−08 | −0.468170277 | KLF4 |
| chr9 | 122119345 | 122119605 | 1.1095E−15 | −0.392000935 | MIR4478 |
| chr9 | 122371737 | 122371962 | 2.82847E−09 | −0.406177722 | PTGS1 |
| chr9 | 123373370 | 123373783 | 1.96776E−11 | 0.398833165 | CRB2 |
| chr9 | 123568713 | 123568775 | 1.36237E−12 | 0.531717381 | DENND1A |
| chr9 | 124267355 | 124267455 | 4.16061E−15 | −0.497151708 | NEK6 |
| chr9 | 125328870 | 125328941 | 6.3755E−09 | −0.479717238 | GAPVD1 |
| chr9 | 125425561 | 125425737 | 7.46442E−12 | 0.480686066 | MAPKAP1 |
| chr9 | 126421902 | 126422248 | 1.61457E−10 | −0.409213162 | MVB12B |
| chr9 | 126662670 | 126662844 | 6.78457E−13 | −0.429147602 | LMX1B |
| chr9 | 127133778 | 127134103 | 6.19469E−15 | 0.507637821 | RALGPS1 |
| chr9 | 127197723 | 127198179 | 1.80293E−14 | 0.435773161 | RALGPS1 |
| chr9 | 127721384 | 127721958 | 1.08444E−12 | −0.386314108 | TTC16 |
| chr9 | 127825253 | 127825349 | 3.70579E−08 | −0.342901255 | ENG |
| chr9 | 128132072 | 128132249 | 8.45991E−14 | 0.433638267 | PTGES2-AS1 |
| chr9 | 128455101 | 128455411 | 1.36869E−10 | −0.3952683 | ODF2 |
| chr9 | 129709470 | 129709878 | 3.27265E−12 | −0.428419489 | PRRX2 |
| chr9 | 130038075 | 130038558 | 2.64282E−12 | −0.445572314 | FNBP1 |
| chr9 | 130194000 | 130194116 | 6.9467E−11 | 0.372333438 | NCS1 |
| chr9 | 130989934 | 130990143 | 2.04015E−07 | −0.411723574 | LAMC3 |
| chr9 | 133038247 | 133038543 | 5.57287E−05 | −0.254842783 | GTF3C5 |
| chr9 | 133129107 | 133129998 | 4.91037E−10 | −0.37840363 | RALGDS |
| chr9 | 133449830 | 133449977 | 9.10841E−12 | −0.389156178 | ADAMTS13 |
| chr9 | 133505957 | 133506655 | 8.45991E−14 | 0.411500911 | MYMK |
| chr9 | 133565717 | 133565917 | 1.2053E−09 | 0.278489457 | ADAMTSL2 |
| chr9 | 133835630 | 133835724 | 7.48307E−08 | 0.323061391 | VAV2 |
| chr9 | 134367841 | 134368120 | 0.004281986 | 0.151676808 | RXRA |
| chr9 | 134438622 | 134438834 | 8.0226E−06 | −0.288657346 | RXRA |
| chr9 | 134637849 | 134637955 | 5.83673E−11 | −0.393381366 | COL5A1 |
| chr9 | 135126542 | 135126666 | 1.15832E−10 | −0.399782169 | OLFM1 |
| chr9 | 135243852 | 135245180 | 6.10121E−12 | −0.411137035 | LINC02907 |
| chr9 | 135438692 | 135438813 | 6.32413E−14 | −0.51221242 | PPP1R26-AS1 |
| chr9 | 135455689 | 135455976 | 1.7567E−15 | 0.479931492 | PPP1R26-AS1 |
| chr9 | 135769831 | 135770602 | 6.78457E−13 | −0.408694039 | KCNT1 |
| chr9 | 135927036 | 135927188 | 3.72444E−09 | −0.391683221 | UBAC1 |
| chr9 | 136017627 | 136018014 | 4.91037E−10 | −0.412215695 | NACC2 |
| chr9 | 136085892 | 136086043 | 1.71082E−05 | −0.247018427 | NACC2 |
| chr9 | 136565261 | 136565445 | 1.08444E−12 | 0.314302535 | NALT1 |
| chr9 | 136694953 | 136695916 | 2.50931E−13 | −0.409928122 | AGPAT2 |
| chr9 | 136821095 | 136821988 | 3.28689E−08 | 0.390047626 | RABL6 |
| chr9 | 137135566 | 137135912 | 4.16801E−13 | −0.449700791 | GRIN1 |
| chr9 | 137197896 | 137198204 | 1.96776E−11 | −0.417309761 | TPRN |
| chr9 | 137217017 | 137217324 | 2.51486E−14 | −0.397067388 | NDOR1 |
| chr9 | 137218200 | 137218451 | 1.07383E−08 | −0.391325023 | NDOR1 |
| chr9 | 137368536 | 137369018 | 7.73912E−10 | 0.42793862 | EXD3 |
| chr9 | 137436307 | 137436441 | 1.08444E−12 | −0.385223903 | ENTPD8 |
| chr9 | 137453787 | 137453851 | 0.153479698 | 0.102049423 | NSMF |
| chr9 | 137505839 | 137506346 | 9.2458E−17 | −0.455260478 | PNPLA7 |
| chr10 | 1464181 | 1464513 | 1.60932E−09 | −0.435249827 | ADARB2 |
| chr10 | 1670568 | 1670796 | 1.62897E−11 | −0.449142893 | ADARB2 |
| chr10 | 1671080 | 1671193 | 1.61457E−10 | −0.423907469 | ADARB2 |
| chr10 | 2225336 | 2225459 | 1.96776E−11 | −0.400804571 | LINC00701 |
| chr10 | 2761465 | 2761714 | 3.4212E−11 | −0.39093415 | LINC02645 |
| chr10 | 2950076 | 2950363 | 3.69832E−16 | −0.490028197 | PFKP |
| chr10 | 3288130 | 3288261 | 3.44869E−14 | −0.456332179 | PITRM1 |
| chr10 | 3333665 | 3333821 | 2.82847E−09 | −0.410954656 | LINC02669 |
| chr10 | 3763125 | 3763331 | 1.28517E−14 | −0.488773918 | KLF6 |
| chr10 | 6052813 | 6052942 | 4.28069E−07 | −0.383683373 | IL2RA |
| chr10 | 6140663 | 6140729 | 8.98223E−10 | −0.427767001 | PFKFB3 |
| chr10 | 6211871 | 6212089 | 3.44869E−14 | 0.42617629 | PFKFB3 |
| chr10 | 6275284 | 6275686 | 2.85148E−11 | −0.435403433 | LINC02649 |
| chr10 | 7576318 | 7576644 | 3.4212E−11 | −0.40258839 | ITIH5 |
| chr10 | 8055348 | 8056042 | 1.84916E−16 | 0.397664428 | GATA3 |
| chr10 | 11204477 | 11204774 | 8.45991E−14 | −0.458311795 | CELF2 |
| chr10 | 11363086 | 11363489 | 7.46442E−12 | −0.412647271 | CELF2 |
| chr10 | 11869555 | 11870537 | 3.24158E−13 | 0.467662551 | PROSER2 |
| chr10 | 11887011 | 11887130 | 2.23571E−10 | −0.472154152 | PROSER2-AS1 |
| chr10 | 11910243 | 11910568 | 1.47656E−13 | −0.460640826 | UPF2 |
| chr10 | 12334276 | 12334416 | 3.24158E−13 | −0.449325063 | CAMK1D |
| chr10 | 12785591 | 12785865 | 1.3454E−11 | −0.418506676 | CAMK1D |
| chr10 | 13729351 | 13729575 | 4.26687E−09 | −0.489165055 | FRMD4A |
| chr10 | 14990717 | 14991541 | 1.84916E−16 | −0.471950039 | MEIG1 |
| chr10 | 17642854 | 17643006 | 5.72228E−10 | −0.40741893 | STAM-DT |
| chr10 | 18096475 | 18096612 | 1.96776E−11 | −0.423342583 | CACNB2 |
| chr10 | 22278135 | 22278272 | 1.80293E−14 | −0.522783922 | LOC100130992 |
| chr10 | 23204834 | 23204989 | 2.64282E−12 | 0.438013874 | C10orf67 |
| chr10 | 24255150 | 24255419 | 1.2053E−09 | −0.420879156 | KIAA1217 |
| chr10 | 24574149 | 24574321 | 2.12644E−12 | −0.456055824 | ARHGAP21 |
| chr10 | 24574149 | 24574321 | 2.12644E−12 | −0.456055824 | ARHGAP21 |
| chr10 | 26439585 | 26439811 | 1.07383E−08 | −0.292444875 | APBB1IP |
| chr10 | 26919776 | 26919845 | 1.70548E−12 | −0.417847431 | FAM238C |
| chr10 | 28897851 | 28898099 | 2.85148E−11 | −0.401500522 | LINC01517 |
| chr10 | 33264449 | 33265059 | 9.10841E−12 | −0.419620558 | NRP1 |
| chr10 | 34933787 | 34933965 | 1.36869E−10 | 0.455081127 | CUL2 |
| chr10 | 34973682 | 34974334 | 6.78457E−13 | −0.454974765 | CUL2 |
| chr10 | 36756509 | 36756709 | 1.70548E−12 | −0.414186987 | ANKRD30A |
| chr10 | 42872955 | 42873149 | 1.70548E−12 | −0.488859697 | LINC02623 |
| chr10 | 43328894 | 43329088 | 8.96843E−15 | −0.414655592 | FXYD4 |
| chr10 | 43405452 | 43405581 | 4.97202E−12 | −0.441324516 | HNRNPF |
| chr10 | 43702238 | 43703170 | 3.24158E−13 | −0.444921688 | ZNF32-AS3 |
| chr10 | 43910402 | 43910550 | 3.24158E−13 | 0.515810255 | LINC00841 |
| chr10 | 43943613 | 43943865 | 2.13847E−09 | −0.429000858 | LINC00841 |
| chr10 | 44828640 | 44828824 | 8.45991E−14 | 0.487313333 | TMEM72-AS1 |
| chr10 | 44879596 | 44879765 | 6.32413E−14 | 0.420753772 | TMEM72-AS1 |
| chr10 | 48973132 | 48973602 | 8.96843E−15 | −0.501142775 | WDFY4 |
| chr10 | 58148625 | 58148785 | 1.1095E−15 | −0.490704432 | IPMK |
| chr10 | 58326977 | 58327260 | 9.2458E−17 | 0.479499845 | LOC112268068 |
| chr10 | 66516832 | 66517383 | 5.58249E−09 | −0.391567441 | CTNNA3 |
| chr10 | 69402348 | 69402638 | 8.96843E−15 | 0.408618927 | HK1 |
| chr10 | 71870172 | 71870251 | 1.99183E−07 | −0.393726738 | PSAP |
| chr10 | 72080814 | 72080990 | 2.62456E−10 | −0.413813153 | SPOCK2 |
| chr10 | 72084046 | 72084426 | 1.04116E−09 | −0.430085816 | SPOCK2 |
| chr10 | 75187836 | 75187943 | 3.27265E−12 | −0.419734856 | SAMD8 |
| chr10 | 77084484 | 77084667 | 1.76361E−10 | 0.613187843 | KCNMA1 |
| chr10 | 78913315 | 78914065 | 2.51486E−14 | −0.469563488 | ZMIZ1-AS1 |
| chr10 | 79139246 | 79139299 | 3.72444E−09 | 0.424936631 | ZMIZ1 |
| chr10 | 79401565 | 79401967 | 3.27265E−12 | 0.46374399 | ZCCHC24 |
| chr10 | 79407882 | 79408010 | 9.10841E−12 | 0.492236284 | ZCCHC24 |
| chr10 | 80206932 | 80207200 | 1.15832E−10 | −0.412434854 | LINC00857 |
| chr10 | 93060307 | 93061590 | 1.1095E−15 | 0.452031144 | CYP26C1 |
| chr10 | 93069204 | 93069437 | 1.08444E−12 | 0.409573007 | CYP26C1 |
| chr10 | 93180570 | 93180900 | 3.69832E−16 | 0.469626581 | CYP26A1 |
| chr10 | 93567842 | 93568390 | 1.36237E−12 | 0.443016601 | FFAR4 |
| chr10 | 96369989 | 96370172 | 6.47206E−16 | −0.424803549 | TLL2 |
| chr10 | 99913544 | 99914123 | 0.000133983 | −0.240962966 | DNMBP |
| chr10 | 100187002 | 100187100 | 5.83673E−11 | −0.445669003 | ERLIN1 |
| chr10 | 100826649 | 100826930 | 2.57927E−08 | 0.394512359 | PAX2 |
| chr10 | 100827029 | 100827735 | 8.38991E−08 | 0.350443482 | PAX2 |
| chr10 | 100827888 | 100828592 | 3.72444E−09 | 0.37488566 | PAX2 |
| chr10 | 100829670 | 100829862 | 1.15832E−10 | 0.371846595 | PAX2 |
| chr10 | 103479067 | 103479385 | 5.32836E−13 | −0.385106736 | CALHM3 |
| chr10 | 103616866 | 103617004 | 3.24158E−13 | 0.490164044 | SH3PXD2A |
| chr10 | 103663897 | 103664067 | 1.71857E−06 | −0.30752633 | SH3PXD2A |
| chr10 | 103747209 | 103747387 | 2.62456E−10 | −0.420269345 | SH3PXD2A |
| chr10 | 108735294 | 108735399 | 4.69687E−14 | −0.407132289 | LINC01435 |
| chr10 | 110460549 | 110460772 | 2.46079E−09 | −0.338127092 | DUSP5 |
| chr10 | 111078588 | 111079369 | 1.7567E−15 | 0.409388969 | ADRA2A |
| chr10 | 115826590 | 115826736 | 8.45991E−14 | −0.474131669 | ATRNL1 |
| chr10 | 117804285 | 117804572 | 6.47206E−16 | −0.404587163 | RAB11FIP2 |
| chr10 | 121303885 | 121304073 | 1.61457E−10 | −0.432717726 | FGFR2 |
| chr10 | 122018918 | 122019439 | 4.69687E−14 | −0.420070784 | TACC2 |
| chr10 | 122474284 | 122474565 | 5.28487E−08 | 0.324643266 | HTRA1 |
| chr10 | 124006200 | 124006300 | 1.15832E−10 | 0.410133193 | CHST15 |
| chr10 | 124381401 | 124381815 | 1.39359E−09 | −0.403843191 | OAT |
| chr10 | 124560633 | 124560763 | 1.36237E−12 | 0.486055651 | LHPP |
| chr10 | 124646505 | 124646645 | 4.09634E−11 | 0.463308164 | FAM53B |
| chr10 | 124703346 | 124703727 | 1.84916E−16 | 0.442621885 | FAM53B |
| chr10 | 124999930 | 125000137 | 3.44869E−14 | 0.492597578 | CTBP2 |
| chr10 | 127024657 | 127024875 | 4.16061E−15 | 0.457349392 | DOCK1 |
| chr10 | 128010621 | 128010887 | 8.25256E−11 | −0.44759429 | PTPRE |
| chr10 | 128047110 | 128047674 | 4.69687E−14 | −0.449326917 | PTPRE |
| chr10 | 129034093 | 129034155 | 1.62897E−11 | −0.463270317 | LINC02667 |
| chr10 | 129396152 | 129396532 | 3.27265E−12 | −0.481320661 | MGMT |
| chr10 | 129531513 | 129531690 | 6.9467E−11 | −0.448339974 | MGMT |
| chr10 | 130930064 | 130930296 | 1.70548E−12 | −0.420528008 | MIR378C |
| chr10 | 131029682 | 131029921 | 8.59398E−13 | −0.414359033 | TCERG1L |
| chr10 | 131136936 | 131137439 | 6.10121E−12 | −0.371673261 | TCERG1L |
| chr10 | 131322623 | 131322953 | 8.96843E−15 | −0.420711002 | TCERG1L |
| chr10 | 131431761 | 131432073 | 9.10841E−12 | −0.447051303 | TCERG1L |
| chr10 | 132107102 | 132107512 | 6.10121E−12 | −0.421236926 | JAKMIP3 |
| chr10 | 132282105 | 132282246 | 8.59398E−13 | −0.422318448 | STK32C |
| chr10 | 132398731 | 132398834 | 3.27265E−12 | −0.409126879 | PWWP2B |
| chr10 | 132520446 | 132520595 | 1.84916E−16 | 0.528766618 | LOC107984282 |
| chr10 | 132713446 | 132713766 | 1.57179E−08 | −0.301576187 | INPP5A |
| chr10 | 132714263 | 132714587 | 4.7463E−07 | −0.378591565 | INPP5A |
| chr10 | 132784304 | 132785308 | 1.80293E−14 | 0.394227518 | NKX6-2 |
| chr10 | 132796894 | 132797398 | 2.23571E−10 | −0.422147459 | NKX6-2 |
| chr10 | 132916437 | 132916700 | 8.25256E−11 | −0.399366812 | CFAP46 |
| chr10 | 132919406 | 132920318 | 3.30121E−06 | −0.390526288 | CFAP46 |
| chr10 | 133013742 | 133013824 | 1.61457E−10 | −0.466858456 | LINC01168 |
| chr10 | 133030218 | 133030290 | 6.47206E−16 | −0.456564548 | ADGRA1 |
| chr10 | 133131979 | 133132168 | 2.62456E−10 | −0.438158614 | ADGRA1 |
| chr10 | 133165216 | 133165494 | 5.32836E−13 | −0.387823871 | KNDC1 |
| chr10 | 133197423 | 133197856 | 8.59398E−13 | −0.447603582 | KNDC1 |
| chr10 | 133205115 | 133205706 | 2.37133E−11 | −0.452703539 | KNDC1 |
| chr10 | 133240837 | 133241677 | 3.27265E−12 | −0.43625189 | VENTX |
| chr10 | 133374220 | 133374377 | 3.4212E−11 | −0.41720533 | ECHS1 |
| chr10 | 133465212 | 133465602 | 1.1095E−15 | 0.41770453 | SCART1 |
| chr10 | 133640598 | 133640709 | 1.74047E−05 | 0.365106555 | FRG2B |
| chr11 | 267752 | 268048 | 6.32413E−14 | −0.446415805 | NLRP6 |
| chr11 | 393949 | 394380 | 6.10121E−12 | −0.413519247 | PKP3 |
| chr11 | 1863154 | 1863237 | 1.9296E−13 | −0.434341836 | LSP1 |
| chr11 | 2257177 | 2257558 | 1.96776E−11 | −0.365766655 | ASCL2 |
| chr11 | 2407100 | 2407342 | 9.10841E−12 | −0.376071442 | TRPM5 |
| chr11 | 2575001 | 2575231 | 9.78564E−11 | −0.401330442 | KCNQ1 |
| chr11 | 3476696 | 3477351 | 2.12644E−12 | −0.43626558 | LOC105376526 |
| chr11 | 6497254 | 6497348 | 2.77374E−15 | 0.454843776 | TIMM10B |
| chr11 | 6630015 | 6630876 | 1.61457E−10 | −0.404780775 | DCHS1 |
| chr11 | 8057262 | 8057392 | 8.25256E−11 | 0.390223887 | TUB |
| chr11 | 9572198 | 9572394 | 1.3454E−11 | −0.445986739 | WEE1 |
| chr11 | 10633234 | 10633371 | 2.77374E−15 | −0.493746992 | IRAG1 |
| chr11 | 10693627 | 10693899 | 3.07638E−10 | 0.399869921 | IRAG1 |
| chr11 | 11973632 | 11973736 | 8.58387E−08 | 0.339074993 | DKK3 |
| chr11 | 15941266 | 15941452 | 6.47206E−16 | 0.435363513 | SOX6 |
| chr11 | 18209094 | 18209378 | 9.2458E−17 | 0.438887266 | SLC25A51P4 |
| chr11 | 33906723 | 33906878 | 6.9467E−11 | −0.401788852 | LMO2 |
| chr11 | 34155371 | 34155596 | 2.12644E−12 | 0.437591197 | ABTB2 |
| chr11 | 34602158 | 34602520 | 1.2053E−09 | −0.396561527 | EHF |
| chr11 | 45161324 | 45161972 | 4.88309E−09 | 0.329565726 | PRDM11 |
| chr11 | 46366283 | 46366474 | 0.00201679 | −0.216988984 | DGKZ |
| chr11 | 47194298 | 47194653 | 9.2458E−17 | −0.410982274 | PACSIN3 |
| chr11 | 47589586 | 47590433 | 9.2458E−17 | 0.521404617 | C1QTNF4 |
| chr11 | 47853918 | 47854090 | 6.19469E−15 | −0.450941339 | NUP160 |
| chr11 | 60054002 | 60054173 | 4.04042E−12 | −0.387813088 | MS4A3 |
| chr11 | 61718760 | 61719026 | 4.26687E−09 | 0.404284242 | DAGLA |
| chr11 | 63435405 | 63435631 | 1.36869E−10 | −0.450263101 | SLC22A9 |
| chr11 | 63664463 | 63664769 | 6.10121E−12 | −0.405366416 | ATL3 |
| chr11 | 64017964 | 64018778 | 1.47656E−13 | −0.392642892 | MACROD1 |
| chr11 | 64101565 | 64101689 | 0.000249389 | 0.275637239 | MACROD1 |
| chr11 | 64554466 | 64555198 | 3.69832E−16 | 0.406220006 | SLC22A11 |
| chr11 | 64759797 | 64759918 | 2.62456E−10 | 0.372583052 | PYGM |
| chr11 | 64843019 | 64843649 | 1.47656E−13 | −0.429049283 | CDC42BPG |
| chr11 | 65047190 | 65047964 | 6.19469E−15 | 0.458073683 | NAALADL1 |
| chr11 | 65183169 | 65183877 | 6.10121E−12 | −0.465568815 | CAPN1 |
| chr11 | 65378458 | 65378987 | 5.72228E−10 | −0.419021765 | SLC25A45 |
| chr11 | 66543756 | 66543853 | 1.10836E−11 | 0.432220397 | ZDHHC24 |
| chr11 | 66718088 | 66718886 | 4.16801E−13 | −0.411645051 | SPTBN2 |
| chr11 | 66743890 | 66744152 | 2.64282E−12 | −0.427066919 | C11orf80 |
| chr11 | 67530067 | 67530223 | 4.69687E−14 | −0.417328573 | CABP2 |
| chr11 | 67726957 | 67727651 | 1.47656E−13 | −0.434034638 | ALDH3B2 |
| chr11 | 67848319 | 67848974 | 4.04042E−12 | −0.45582226 | FAM86C2P |
| chr11 | 67978391 | 67978950 | 3.69832E−16 | −0.420213073 | UNC93B1 |
| chr11 | 68008652 | 68008940 | 1.70548E−12 | −0.403530282 | ALDH3B1 |
| chr11 | 68373555 | 68373654 | 4.04042E−12 | 0.431829979 | LRP5 |
| chr11 | 68413197 | 68413330 | 1.08444E−12 | 0.425622341 | LRP5 |
| chr11 | 68890278 | 68890434 | 1.84916E−16 | 0.435459469 | MRPL21 |
| chr11 | 69014504 | 69014763 | 8.96843E−15 | 0.439790977 | MRGPRF-AS1 |
| chr11 | 69251759 | 69252386 | 2.64282E−12 | −0.401497056 | MYEOV |
| chr11 | 69301933 | 69302307 | 1.28517E−14 | −0.425285454 | MYEOV |
| chr11 | 69391446 | 69391798 | 4.89446E−11 | −0.392477129 | LOC102724265 |
| chr11 | 69465727 | 69465949 | 2.13847E−09 | −0.388833038 | LINC01488 |
| chr11 | 69546788 | 69546971 | 2.12644E−12 | −0.378192935 | LINC01488 |
| chr11 | 69940110 | 69940151 | 4.89446E−11 | 0.426125708 | ANO1 |
| chr11 | 70615441 | 70615831 | 1.15832E−10 | −0.453751338 | SHANK2 |
| chr11 | 71140454 | 71141101 | 1.9296E−13 | −0.473787674 | SHANK2 |
| chr11 | 71615079 | 71615751 | 9.2458E−17 | −0.42475581 | KRTAP5-11 |
| chr11 | 71736977 | 71737284 | 6.9467E−11 | −0.393183272 | ALG1L9P |
| chr11 | 71745603 | 71746258 | 2.77374E−15 | −0.555390711 | ALG1L9P |
| chr11 | 71765298 | 71765321 | 8.2251E−08 | 0.371868244 | ALG1L9P |
| chr11 | 71889636 | 71889876 | 1.47656E−13 | 0.47916879 | LOC100133315 |
| chr11 | 73228105 | 73228384 | 8.59398E−13 | −0.548307544 | P2RY2 |
| chr11 | 73981110 | 73981477 | 5.32836E−13 | −0.440438273 | UCP2 |
| chr11 | 73981128 | 73981495 | 5.83673E−11 | −0.438625789 | UCP2 |
| chr11 | 74535081 | 74535497 | 1.3454E−11 | −0.48008307 | POLD3 |
| chr11 | 76292298 | 76292426 | 6.78457E−13 | −0.472893096 | THAP12 |
| chr11 | 76582832 | 76583179 | 1.08444E−12 | −0.460609048 | EMSY |
| chr11 | 80699254 | 80699883 | 6.78457E−13 | −0.529976685 | LINC02720 |
| chr11 | 84212751 | 84212954 | 5.82391E−07 | 0.41592213 | DLG2 |
| chr11 | 89787081 | 89787203 | 1.08444E−12 | 0.42784435 | TRIM49 |
| chr11 | 94089075 | 94089454 | 1.10836E−11 | −0.393267835 | HEPHL1 |
| chr11 | 94650830 | 94651002 | 3.27265E−12 | 0.40489269 | PIWIL4-AS1 |
| chr11 | 95037571 | 95038029 | 6.9467E−11 | 0.399167715 | KDM4E |
| chr11 | 95107752 | 95108047 | 1.96776E−11 | −0.431739325 | ENDOD1 |
| chr11 | 111919199 | 111919301 | 1.36869E−10 | −0.458170165 | C11orf52 |
| chr11 | 112499849 | 112500109 | 8.25256E−11 | −0.372367761 | LINC02763 |
| chr11 | 112864858 | 112865069 | 1.10836E−11 | 0.46150752 | LOC101928847 |
| chr11 | 115582403 | 115582635 | 1.28517E−14 | −0.456781718 | CADM1 |
| chr11 | 117457516 | 117457955 | 8.59398E−13 | −0.424712393 | DSCAML1 |
| chr11 | 117872798 | 117872908 | 8.45991E−14 | 0.499854007 | FXYD6 |
| chr11 | 118624770 | 118624982 | 1.85291E−09 | −0.416143967 | PHLDB1 |
| chr11 | 119376059 | 119376290 | 0.000841222 | 0.181222324 | USP2 |
| chr11 | 120496972 | 120497256 | 2.12644E−12 | −0.509734879 | ARHGEF12 |
| chr11 | 124748511 | 124748790 | 1.84916E−16 | 0.450875601 | VSIG2 |
| chr11 | 125409661 | 125409783 | 1.47656E−13 | −0.39875108 | PKNOX2 |
| chr11 | 126165891 | 126166002 | 1.84916E−16 | 0.543833967 | RPUSD4 |
| chr11 | 127363138 | 127363393 | 1.88966E−06 | −0.395386206 | LINC02712 |
| chr11 | 129316122 | 129316233 | 6.10121E−12 | −0.403168077 | ARHGAP32 |
| chr11 | 130648624 | 130648889 | 6.3755E−09 | 0.414784813 | MIR8052 |
| chr11 | 131869214 | 131869460 | 1.3454E−11 | −0.40432192 | NTM |
| chr11 | 133974189 | 133974369 | 7.46442E−12 | −0.416706726 | IGSF9B |
| chr11 | 134038508 | 134038572 | 1.12059E−13 | −0.426631279 | LINC02731 |
| chr11 | 134701723 | 134701926 | 1.80293E−14 | −0.406593273 | LINC02714 |
| chr12 | 1576926 | 1577102 | 1.08444E−12 | 0.482879617 | WNT5B |
| chr12 | 1576926 | 1577102 | 1.08444E−12 | 0.482879617 | WNT5B |
| chr12 | 2633872 | 2634073 | 4.09634E−11 | −0.424754066 | CACNA1C |
| chr12 | 2858783 | 2859461 | 1.47656E−13 | 0.419712084 | ITFG2 |
| chr12 | 3252439 | 3252855 | 1.9296E−13 | 0.521373304 | TSPAN9 |
| chr12 | 3311257 | 3311512 | 1.12059E−13 | −0.480584917 | LOC100128253 |
| chr12 | 6088338 | 6088404 | 1.3454E−11 | −0.480904319 | VWF |
| chr12 | 6266852 | 6267075 | 5.32836E−13 | −0.390127973 | CD9 |
| chr12 | 6282094 | 6282249 | 2.91289E−08 | −0.402336436 | PLEKHG6 |
| chr12 | 6554973 | 6555437 | 1.9296E−13 | 0.518027004 | IFFO1 |
| chr12 | 6555602 | 6555857 | 1.7567E−15 | 0.542809973 | IFFO1 |
| chr12 | 6555932 | 6556221 | 1.1095E−15 | 0.56266995 | IFFO1 |
| chr12 | 6963221 | 6964182 | 8.45991E−14 | −0.448142731 | MIR200C |
| chr12 | 7639906 | 7640147 | 0.000124809 | 0.267948142 | APOBEC1 |
| chr12 | 7705207 | 7705380 | 1.36237E−12 | −0.464290018 | DPPA3 |
| chr12 | 7705962 | 7706372 | 8.25256E−11 | −0.518671058 | DPPA3 |
| chr12 | 7872696 | 7873290 | 1.17715E−07 | 0.368334043 | SLC2A14 |
| chr12 | 8285786 | 8286119 | 6.32413E−14 | −0.474213813 | LINC00937 |
| chr12 | 8682494 | 8682568 | 6.19469E−15 | −0.440737103 | RIMKLB |
| chr12 | 11547260 | 11547675 | 3.27265E−12 | −0.53799306 | LINC01252 |
| chr12 | 13377104 | 13377329 | 6.9467E−11 | −0.425469571 | LINC01559 |
| chr12 | 24902038 | 24902175 | 5.83673E−11 | −0.473813292 | BCAT1 |
| chr12 | 30169832 | 30170613 | 1.7567E−15 | 0.426331505 | TMTC1 |
| chr12 | 30201273 | 30201731 | 2.77374E−15 | 0.416921484 | TMTC1 |
| chr12 | 30409900 | 30410109 | 4.20778E−10 | 0.452159031 | IPO8 |
| chr12 | 31103630 | 31103814 | 6.10121E−12 | −0.441870624 | DDX11 |
| chr12 | 34107874 | 34108698 | 3.27265E−12 | 0.417248271 | ALG10 |
| chr12 | 34205008 | 34205143 | 6.32413E−14 | −0.478932528 | ALG10 |
| chr12 | 34326062 | 34326104 | 3.93843E−09 | −0.410564571 | ALG10 |
| chr12 | 34335373 | 34335427 | 4.97202E−12 | −0.427843019 | ALG10 |
| chr12 | 34337462 | 34337910 | 6.19469E−15 | −0.408893947 | ALG10 |
| chr12 | 34338022 | 34338335 | 3.69832E−16 | −0.405212689 | ALG10 |
| chr12 | 34398905 | 34399054 | 4.16061E−15 | −0.421739412 | ALG10 |
| chr12 | 46382382 | 46382658 | 6.10121E−12 | −0.415870847 | LOC100288798 |
| chr12 | 47737525 | 47737681 | 1.85622E−09 | 0.348908942 | RAPGEF3 |
| chr12 | 48553902 | 48554151 | 4.97202E−12 | −0.410937969 | LALBA |
| chr12 | 49336171 | 49336352 | 1.08444E−12 | 0.404888986 | C1QL4 |
| chr12 | 49955982 | 49956600 | 4.97202E−12 | −0.440715166 | AQP2 |
| chr12 | 51392183 | 51392881 | 1.3454E−11 | −0.397802949 | SLC4A8 |
| chr12 | 52191551 | 52191964 | 1.28517E−14 | −0.414964994 | LINC00592 |
| chr12 | 52258271 | 52258911 | 9.2458E−17 | 0.517921314 | KRT87P |
| chr12 | 52519992 | 52520514 | 6.65919E−10 | −0.387412316 | KRT5 |
| chr12 | 52902412 | 52902869 | 5.83673E−11 | −0.437258154 | KRT8 |
| chr12 | 53601603 | 53601754 | 1.28455E−08 | 0.48040175 | ATF7-NPFF |
| chr12 | 54019387 | 54020245 | 2.12644E−12 | 0.454672595 | HOXC6 |
| chr12 | 54021198 | 54021364 | 1.60932E−09 | 0.439965206 | HOXC6 |
| chr12 | 54030826 | 54031635 | 6.9467E−11 | 0.391425253 | HOXC5 |
| chr12 | 54288511 | 54288826 | 2.77374E−15 | −0.457218357 | HNRNPA1 |
| chr12 | 54417977 | 54418138 | 4.69687E−14 | 0.419587956 | ITGA5 |
| chr12 | 55935793 | 55936120 | 1.04116E−09 | 0.406420201 | DGKA |
| chr12 | 56685545 | 56685692 | 8.98223E−10 | −0.397626793 | PTGES3 |
| chr12 | 56689727 | 56689849 | 1.39359E−09 | −0.414878886 | PTGES3 |
| chr12 | 56780580 | 56780718 | 1.36869E−10 | −0.472684089 | HSD17B6 |
| chr12 | 57135917 | 57136139 | 1.1095E−15 | 0.522114836 | LRP1 |
| chr12 | 57183472 | 57183507 | 4.20778E−10 | −0.427579047 | LRP1 |
| chr12 | 57245530 | 57245666 | 3.69832E−16 | −0.49524378 | STAC3 |
| chr12 | 66303348 | 66303846 | 1.3454E−11 | −0.47308036 | HELB |
| chr12 | 70429387 | 70429473 | 2.37133E−11 | −0.447843348 | KCNMB4 |
| chr12 | 75207125 | 75208134 | 2.51486E−14 | 0.343605626 | KCNC2 |
| chr12 | 80708167 | 80708738 | 1.36237E−12 | 0.383633496 | MYF6 |
| chr12 | 94186996 | 94187077 | 5.65844E−06 | 0.325123962 | PLXNC1 |
| chr12 | 95472692 | 95472952 | 9.2458E−17 | −0.398722605 | METAP2 |
| chr12 | 95649670 | 95649807 | 5.72228E−10 | −0.412817287 | PGAM1P5 |
| chr12 | 96219828 | 96219990 | 3.27265E−12 | −0.522389646 | ELK3 |
| chr12 | 98517884 | 98518070 | 6.1791E−06 | −0.352516915 | TMPO |
| chr12 | 104115287 | 104115423 | 9.43988E−09 | −0.394575319 | NFYB |
| chr12 | 108911583 | 108912213 | 1.28517E−14 | −0.409789495 | SVOP |
| chr12 | 110345279 | 110345436 | 1.94812E−12 | −0.504554239 | ATP2A2 |
| chr12 | 110500900 | 110500951 | 1.12059E−13 | −0.437055018 | VPS29 |
| chr12 | 113890009 | 113890480 | 5.32836E−13 | 0.485554434 | RBM19 |
| chr12 | 116536913 | 116537606 | 6.9467E−11 | −0.388914667 | LINC00173 |
| chr12 | 117803558 | 117803777 | 5.58249E−09 | −0.385506561 | KSR2 |
| chr12 | 119803868 | 119804415 | 7.46442E−12 | 0.373128369 | CIT |
| chr12 | 121353600 | 121353736 | 7.73912E−10 | −0.437853781 | ANAPC5 |
| chr12 | 122060111 | 122060340 | 2.64282E−12 | 0.424178373 | BCL7A |
| chr12 | 124227931 | 124228531 | 1.3454E−11 | −0.423108754 | ZNF664-RFLNA |
| chr12 | 124327100 | 124327365 | 4.89446E−11 | 0.424939767 | NCOR2 |
| chr12 | 124411438 | 124411609 | 5.32836E−13 | 0.386356474 | NCOR2 |
| chr12 | 124699924 | 124700256 | 5.58249E−09 | −0.373178959 | SCARB1 |
| chr12 | 124758172 | 124758758 | 3.95894E−06 | −0.271143123 | SCARB1 |
| chr12 | 131035128 | 131035468 | 2.23571E−10 | −0.366316947 | ADGRD1 |
| chr12 | 132215025 | 132215373 | 1.64079E−07 | −0.31370628 | GALNT9 |
| chr12 | 132219413 | 132219698 | 3.27265E−12 | −0.451142046 | GALNT9 |
| chr12 | 132288689 | 132288968 | 1.36237E−12 | −0.488305832 | GALNT9 |
| chr12 | 132837703 | 132838203 | 8.25256E−11 | 0.42507822 | CHFR |
| chr13 | 20717007 | 20717159 | 5.32836E−13 | −0.411901678 | IL17D |
| chr13 | 21319112 | 21319212 | 9.2458E−17 | 0.466909262 | GRK6P1 |
| chr13 | 21475013 | 21475149 | 5.71001E−09 | −0.466598143 | ZDHHC20 |
| chr13 | 21812207 | 21812566 | 9.78564E−11 | −0.338065556 | LINC00424 |
| chr13 | 22220414 | 22220715 | 2.37133E−11 | −0.442734401 | LINC00540 |
| chr13 | 23139104 | 23139230 | 6.9467E−11 | −0.419577717 | SGCG |
| chr13 | 24627272 | 24627296 | 1.17715E−07 | 0.378194799 | TPTE2P6 |
| chr13 | 37069467 | 37070177 | 1.80293E−14 | −0.447394481 | SUPT20H |
| chr13 | 42969056 | 42969358 | 6.32413E−14 | −0.414344541 | EPSTI1 |
| chr13 | 49220790 | 49221368 | 9.78564E−11 | 0.329370047 | MLNR |
| chr13 | 50365489 | 50365866 | 1.70548E−12 | −0.459891747 | DLEU1 |
| chr13 | 57632445 | 57634006 | 2.37133E−11 | 0.325899755 | PCDH17 |
| chr13 | 73235176 | 73235495 | 1.39359E−09 | −0.419245618 | KLF5 |
| chr13 | 77291491 | 77291624 | 1.3454E−11 | −0.432110888 | MYCBP2 |
| chr13 | 98279875 | 98280241 | 4.16801E−13 | −0.49999718 | FARP1 |
| chr13 | 109705043 | 109705583 | 1.62897E−11 | −0.461942981 | LINC00676 |
| chr13 | 110045124 | 110045563 | 6.9467E−11 | −0.407618357 | LINC00396 |
| chr13 | 110046585 | 110046724 | 1.38562E−08 | −0.407500997 | LINC00396 |
| chr13 | 110116186 | 110116262 | 4.91037E−10 | 0.418196937 | COL4A1 |
| chr13 | 110123135 | 110123752 | 4.04042E−12 | −0.417978808 | COL4A1 |
| chr13 | 111121648 | 111121823 | 1.90143E−10 | −0.401746189 | ARHGEF7 |
| chr13 | 112842387 | 112842564 | 2.07662E−06 | 0.346702933 | ATP11A |
| chr13 | 112967724 | 112967994 | 3.6005E−10 | −0.391280858 | MCF2L |
| chr13 | 112994125 | 112994775 | 6.9467E−11 | −0.418111609 | MCF2L |
| chr13 | 112995542 | 112996034 | 3.24748E−09 | 0.39137281 | MCF2L |
| chr13 | 113051279 | 113051455 | 3.44869E−14 | 0.491232226 | MCF2L |
| chr13 | 113820789 | 113821064 | 4.16801E−13 | 0.440327056 | GAS6-AS1 |
| chr13 | 113821122 | 113821270 | 1.10836E−11 | 0.454155291 | GAS6-AS1 |
| chr13 | 113927859 | 113928367 | 1.47656E−13 | 0.439219388 | LINC00565 |
| chr13 | 113970077 | 113970662 | 1.60932E−09 | −0.391964801 | C13orf46 |
| chr13 | 113978904 | 113979410 | 1.41907E−06 | −0.323500122 | RASA3 |
| chr13 | 113981291 | 113981869 | 6.78457E−13 | −0.389015533 | RASA3 |
| chr14 | 24071482 | 24071612 | 1.64079E−07 | 0.287596651 | CPNE6 |
| chr14 | 28774427 | 28774557 | 1.96776E−11 | 0.313828335 | LINC01551 |
| chr14 | 34677497 | 34677700 | 4.16061E−15 | −0.466779721 | CFL2 |
| chr14 | 35294136 | 35294320 | 1.95155E−10 | −0.448855565 | PRORP |
| chr14 | 36509159 | 36509191 | 2.23571E−10 | 0.374972487 | SFTA3 |
| chr14 | 36583332 | 36584402 | 6.47206E−16 | 0.389733772 | NKX2-8 |
| chr14 | 54776434 | 54776550 | 1.07383E−08 | −0.307801626 | SAMD4A |
| chr14 | 55240985 | 55241156 | 1.84916E−16 | 0.417027258 | FBXO34 |
| chr14 | 56809111 | 56809118 | 1.47656E−13 | 0.437650196 | OTX2 |
| chr14 | 64702946 | 64703263 | 8.59398E−13 | −0.46439231 | PLEKHG3 |
| chr14 | 68628308 | 68628619 | 4.69687E−14 | −0.444614116 | RAD51B |
| chr14 | 72679685 | 72680170 | 1.5621E−06 | 0.238297409 | DPF3 |
| chr14 | 73719050 | 73719566 | 6.9467E−11 | 0.407370932 | MIDEAS |
| chr14 | 74573813 | 74573962 | 2.12644E−12 | −0.462613642 | LTBP2 |
| chr14 | 75980146 | 75980342 | 1.15832E−10 | 0.422818085 | TGFB3 |
| chr14 | 80973260 | 80973381 | 1.10836E−11 | −0.426448129 | TSHR |
| chr14 | 84017767 | 84017922 | 1.22038E−08 | −0.391734468 | LINC02301 |
| chr14 | 89367232 | 89367508 | 6.47206E−16 | 0.42625794 | FOXN3 |
| chr14 | 95701315 | 95701447 | 6.78457E−13 | 0.376308126 | TCL1A |
| chr14 | 97057810 | 97057961 | 9.78564E−11 | −0.422555957 | LINC02304 |
| chr14 | 99066185 | 99066557 | 1.9296E−13 | −0.418961873 | BCL11B |
| chr14 | 99624880 | 99625184 | 3.24158E−13 | −0.416468177 | HHIPL1 |
| chr14 | 100390476 | 100390936 | 5.32836E−13 | 0.446904329 | WDR25 |
| chr14 | 100691598 | 100691984 | 9.36133E−05 | −0.239128669 | LINC00523 |
| chr14 | 100712322 | 100712729 | 4.97202E−12 | −0.406455404 | DLK1 |
| chr14 | 101560094 | 101561742 | 4.04042E−12 | 0.321677543 | DIO3OS |
| chr14 | 102215131 | 102215375 | 1.61457E−10 | 0.424804855 | WDR20 |
| chr14 | 103543932 | 103544350 | 3.12829E−07 | 0.264067988 | TRMT61A |
| chr14 | 103879682 | 103879840 | 6.47206E−16 | −0.399513344 | PPP1R13B-DT |
| chr14 | 104225610 | 104225868 | 1.62897E−11 | 0.389263758 | LINC02691 |
| chr14 | 104346181 | 104346269 | 6.19469E−15 | −0.449419434 | LINC02691 |
| chr14 | 104699138 | 104699448 | 7.87923E−07 | 0.33944887 | INF2 |
| chr14 | 104725250 | 104725788 | 4.04042E−12 | 0.360921944 | ADSS1 |
| chr14 | 105469850 | 105470731 | 1.57179E−08 | −0.400866304 | MTA1 |
| chr14 | 105488052 | 105488134 | 3.72444E−09 | −0.379416912 | TEDC1 |
| chr15 | 22238473 | 22238969 | 2.51486E−14 | −0.412657967 | MIR1268A |
| chr15 | 37096379 | 37097279 | 8.59398E−13 | 0.431857047 | MEIS2 |
| chr15 | 39976477 | 39977040 | 7.87923E−07 | 0.2998901 | EIF2AK4 |
| chr15 | 40074969 | 40075388 | 1.9296E−13 | −0.414650204 | SRP14-AS1 |
| chr15 | 40583873 | 40583944 | 3.07638E−10 | −0.424547995 | RPUSD2 |
| chr15 | 43132941 | 43133000 | 1.08444E−12 | −0.428524829 | TMEM62 |
| chr15 | 50885582 | 50885656 | 6.44522E−07 | −0.382890768 | AP4E1 |
| chr15 | 52675629 | 52675831 | 3.6161E−06 | −0.439567085 | FAM214A |
| chr15 | 59993537 | 59993611 | 3.69832E−16 | 0.461360707 | FOXB1 |
| chr15 | 61750751 | 61751115 | 1.36237E−12 | −0.389187342 | LINC02349 |
| chr15 | 62075519 | 62075654 | 9.10841E−12 | −0.431551229 | C2CD4A |
| chr15 | 63597328 | 63597705 | 4.04042E−12 | 0.414705214 | FBXL22 |
| chr15 | 64905631 | 64905684 | 3.6161E−06 | 0.320495125 | PLEKHO2 |
| chr15 | 66729268 | 66729856 | 5.25922E−07 | 0.34921788 | SMAD6 |
| chr15 | 66936168 | 66936902 | 6.19469E−15 | 0.51774334 | LINC02206 |
| chr15 | 67824132 | 67824140 | 0.019379991 | 0.216237112 | SKOR1 |
| chr15 | 70415506 | 70415622 | 5.25922E−07 | −0.303650253 | LINC02205 |
| chr15 | 74208866 | 74209430 | 6.10121E−12 | −0.404177227 | STRA6 |
| chr15 | 74788774 | 74789276 | 2.23571E−10 | −0.389020018 | CSK |
| chr15 | 75106015 | 75106284 | 7.73912E−10 | −0.411219025 | PPCDC |
| chr15 | 76996473 | 76996653 | 2.23571E−10 | 0.379556386 | PSTPIP1 |
| chr15 | 77028328 | 77028582 | 2.91289E−08 | 0.371446869 | PSTPIP1 |
| chr15 | 80871983 | 80872196 | 4.89446E−11 | −0.433208283 | CEMIP |
| chr15 | 85785185 | 85785384 | 4.97202E−12 | −0.419646693 | KLHL25 |
| chr15 | 88542188 | 88542490 | 1.28517E−14 | 0.428124367 | DET1 |
| chr15 | 89445046 | 89445175 | 6.32413E−14 | 0.40770069 | RHCG |
| chr15 | 92103911 | 92104160 | 9.52458E−06 | −0.298912866 | SLCO3A1 |
| chr15 | 92624480 | 92624948 | 0.002516027 | 0.151330681 | FAM174B |
| chr15 | 96052971 | 96053249 | 1.7567E−15 | −0.443817168 | LOC105369212 |
| chr15 | 96052978 | 96053249 | 1.7567E−15 | −0.443325858 | LOC105369212 |
| chr15 | 96341967 | 96342444 | 1.15832E−10 | 0.410627021 | NR2F2 |
| chr15 | 96343656 | 96343961 | 1.36237E−12 | 0.47968718 | NR2F2 |
| chr15 | 96347511 | 96347828 | 1.96776E−11 | 0.436489011 | NR2F2 |
| chr15 | 96367880 | 96368878 | 1.05228E−07 | −0.333683645 | NR2F2 |
| chr15 | 98873946 | 98874158 | 2.85148E−11 | −0.417778126 | IGF1R |
| chr15 | 100022519 | 100022965 | 7.12842E−07 | 0.349584652 | ADAMTS17 |
| chr15 | 100061036 | 100061200 | 6.32413E−14 | −0.425880455 | ADAMTS17 |
| chr15 | 100061036 | 100061200 | 6.32413E−14 | −0.425880455 | ADAMTS17 |
| chr15 | 100276790 | 100277216 | 3.69832E−16 | 0.41348323 | ADAMTS17 |
| chr15 | 101091658 | 101091849 | 6.10121E−12 | 0.467977013 | LRRK1 |
| chr15 | 101437197 | 101437491 | 4.20778E−10 | −0.384977848 | PCSK6 |
| chr15 | 101451661 | 101451771 | 1.62897E−11 | 0.428721453 | PCSK6 |
| chr16 | 782683 | 782964 | 2.37133E−11 | −0.386309203 | RPUSD1 |
| chr16 | 985321 | 985461 | 4.89446E−11 | −0.395299067 | SOX8 |
| chr16 | 990680 | 991421 | 1.9296E−13 | −0.396461248 | SOX8 |
| chr16 | 1017046 | 1017362 | 1.96776E−11 | −0.39088932 | SOX8 |
| chr16 | 1046612 | 1047258 | 1.28517E−14 | −0.411271507 | SSTR5-AS1 |
| chr16 | 1142672 | 1142764 | 4.69687E−14 | −0.435467525 | CACNA1H |
| chr16 | 1532180 | 1532309 | 9.10841E−12 | 0.375541741 | IFT140 |
| chr16 | 1533809 | 1534516 | 3.27265E−12 | 0.433496365 | IFT140 |
| chr16 | 1679805 | 1680350 | 1.36237E−12 | −0.433578143 | JPT2 |
| chr16 | 2081763 | 2081889 | 7.27381E−09 | −0.378218479 | TSC2 |
| chr16 | 2224649 | 2224778 | 1.61457E−10 | −0.440093939 | E4F1 |
| chr16 | 2481030 | 2481266 | 1.15832E−10 | −0.37835775 | TBC1D24 |
| chr16 | 2482603 | 2482971 | 1.80293E−14 | 0.440515651 | TBC1D24 |
| chr16 | 2536752 | 2537243 | 1.84916E−16 | −0.431112454 | PDPK1 |
| chr16 | 3108173 | 3108339 | 4.97202E−12 | −0.402108826 | ZNF205-AS1 |
| chr16 | 3171802 | 3172113 | 4.20778E−10 | 0.396259317 | OR1F1 |
| chr16 | 3295145 | 3295415 | 6.47206E−16 | 0.420544455 | TIGD7 |
| chr16 | 4188435 | 4188597 | 2.12644E−12 | −0.414912346 | SRL |
| chr16 | 4260230 | 4260833 | 4.04042E−12 | −0.432972382 | TFAP4 |
| chr16 | 4263233 | 4263352 | 4.09634E−11 | 0.397858857 | TFAP4 |
| chr16 | 4795665 | 4796544 | 5.83673E−11 | −0.416232021 | ROGDI |
| chr16 | 4892354 | 4892510 | 1.2053E−09 | 0.38290613 | PPL |
| chr16 | 6195438 | 6195644 | 6.19469E−15 | −0.466192064 | RBFOX1 |
| chr16 | 6472824 | 6473099 | 6.19469E−15 | −0.428993316 | RBFOX1 |
| chr16 | 8742312 | 8742735 | 3.44869E−14 | 0.435390638 | ABAT |
| chr16 | 10882668 | 10883143 | 8.45991E−14 | −0.430494862 | CIITA |
| chr16 | 10980247 | 10980629 | 6.19469E−15 | 0.444870081 | CLEC16A |
| chr16 | 11595113 | 11595586 | 2.77374E−15 | 0.41172132 | LITAF |
| chr16 | 11651956 | 11652273 | 4.04042E−12 | −0.421396012 | LITAF |
| chr16 | 27168798 | 27169176 | 4.89446E−11 | −0.400649496 | KDM8 |
| chr16 | 27495317 | 27495488 | 1.07383E−08 | 0.442856668 | GTF3C1 |
| chr16 | 27779840 | 27780035 | 8.29042E−09 | 0.42252761 | KATNIP |
| chr16 | 29074899 | 29074961 | 0.00201679 | 0.299931904 | SNX29P2 |
| chr16 | 29292990 | 29293407 | 9.78564E−11 | −0.383789819 | SNX29P2 |
| chr16 | 30004873 | 30004981 | 4.88309E−09 | −0.428400816 | SULT1A3 |
| chr16 | 30004873 | 30004981 | 4.88309E−09 | −0.428400816 | SULT1A3 |
| chr16 | 30445011 | 30445193 | 2.04015E−07 | −0.384181693 | SEPHS2 |
| chr16 | 30561733 | 30562014 | 6.3755E−09 | −0.370322822 | ZNF764 |
| chr16 | 31042252 | 31042356 | 4.16061E−15 | 0.488482467 | STX4 |
| chr16 | 31130240 | 31130659 | 3.44869E−14 | −0.410272949 | KAT8 |
| chr16 | 31131520 | 31131671 | 1.08444E−12 | −0.440529644 | PRSS8 |
| chr16 | 31476202 | 31477604 | 1.1095E−15 | −0.477224302 | TGFB1I1 |
| chr16 | 46649511 | 46649658 | 3.6005E−10 | −0.397525547 | VPS35 |
| chr16 | 48243712 | 48243907 | 5.83673E−11 | −0.521643321 | ABCC11 |
| chr16 | 48629408 | 48629877 | 3.07638E−10 | −0.383245536 | N4BP1 |
| chr16 | 49510753 | 49511110 | 1.60932E−09 | −0.392327986 | ZNF423 |
| chr16 | 49602571 | 49602936 | 3.28689E−08 | −0.368630196 | ZNF423 |
| chr16 | 49663978 | 49664738 | 1.10836E−11 | 0.356262914 | ZNF423 |
| chr16 | 50585368 | 50585523 | 3.27265E−12 | 0.435688705 | NKD1 |
| chr16 | 50613334 | 50613826 | 6.32413E−14 | 0.435620629 | NKD1 |
| chr16 | 50719639 | 50719815 | 3.4212E−11 | −0.407119809 | NOD2 |
| chr16 | 54382005 | 54382176 | 8.96843E−15 | 0.450430502 | LINC02140 |
| chr16 | 55332051 | 55332195 | 1.83024E−07 | 0.271496347 | IRX6 |
| chr16 | 56627594 | 56627778 | 3.24158E−13 | −0.423006952 | MT1E |
| chr16 | 57115531 | 57115700 | 2.81406E−07 | 0.300708924 | CPNE2 |
| chr16 | 57619980 | 57620874 | 1.36237E−12 | −0.395470646 | ADGRG1 |
| chr16 | 57623236 | 57623492 | 2.85148E−11 | −0.362625161 | ADGRG1 |
| chr16 | 57797403 | 57798380 | 1.28517E−14 | −0.456003627 | LOC388282 |
| chr16 | 57893945 | 57894234 | 1.43926E−10 | −0.405831358 | CNGB1 |
| chr16 | 64573514 | 64573701 | 1.08444E−12 | −0.501663538 | CDH11 |
| chr16 | 64573514 | 64573701 | 1.08444E−12 | −0.501663538 | CDH11 |
| chr16 | 65755607 | 65755675 | 2.23571E−10 | 0.409211116 | LINC00922 |
| chr16 | 67110971 | 67111110 | 5.83673E−11 | −0.432125586 | PHAF1 |
| chr16 | 67406302 | 67406381 | 1.84916E−16 | 0.439973968 | ZDHHC1 |
| chr16 | 68361509 | 68361659 | 3.70579E−08 | 0.327605377 | SMPD3 |
| chr16 | 68745041 | 68745468 | 1.15832E−10 | −0.376278822 | CDH1 |
| chr16 | 70696862 | 70697593 | 9.10841E−12 | −0.397867299 | VAC14 |
| chr16 | 70725486 | 70726253 | 4.20778E−10 | 0.402431217 | VAC14 |
| chr16 | 72875707 | 72876008 | 4.97202E−12 | −0.441439288 | ZFHX3 |
| chr16 | 74839060 | 74839196 | 1.08444E−12 | −0.422433036 | WDR59 |
| chr16 | 75115124 | 75115213 | 3.6005E−10 | −0.43077132 | LDHD |
| chr16 | 80027227 | 80027528 | 2.37133E−11 | −0.465970582 | DYNLRB2-AS1 |
| chr16 | 81651231 | 81651520 | 2.57927E−08 | 0.369045829 | CMIP |
| chr16 | 84398819 | 84399009 | 1.80293E−14 | 0.453203899 | ATP2C2 |
| chr16 | 84818877 | 84819437 | 4.69687E−14 | −0.40114381 | CRISPLD2 |
| chr16 | 84842247 | 84842521 | 1.70548E−12 | −0.427664482 | CRISPLD2 |
| chr16 | 85082725 | 85083028 | 2.64282E−12 | −0.429213532 | KIAA0513 |
| chr16 | 85112342 | 85112529 | 3.44869E−14 | −0.404896504 | CIBAR2 |
| chr16 | 85335491 | 85335766 | 2.50931E−13 | −0.395608653 | GSE1 |
| chr16 | 85360309 | 85360597 | 1.36237E−12 | −0.424844354 | GSE1 |
| chr16 | 85416434 | 85416526 | 1.28517E−14 | −0.443950052 | GSE1 |
| chr16 | 85464598 | 85465180 | 1.06021E−06 | −0.310927683 | GSE1 |
| chr16 | 85483578 | 85483778 | 8.25256E−11 | −0.406697032 | GSE1 |
| chr16 | 85521010 | 85521195 | 4.16801E−13 | −0.475709241 | GSE1 |
| chr16 | 85570633 | 85571125 | 2.50931E−13 | −0.445552508 | GSE1 |
| chr16 | 85614473 | 85615346 | 4.97202E−12 | −0.427087912 | GSE1 |
| chr16 | 86531167 | 86531437 | 1.12059E−13 | 0.445504076 | MTHFSD |
| chr16 | 87013402 | 87013533 | 4.11965E−08 | 0.350099785 | LINC02181 |
| chr16 | 87867024 | 87867520 | 4.04042E−12 | −0.42466602 | SLC7A5 |
| chr16 | 87867836 | 87867974 | 8.45991E−14 | −0.438836441 | SLC7A5 |
| chr16 | 88130745 | 88131423 | 4.09634E−11 | −0.418368859 | LOC400553 |
| chr16 | 88234472 | 88234638 | 4.97202E−12 | 0.457226864 | LINC02182 |
| chr16 | 88267044 | 88267554 | 4.69895E−08 | −0.368160583 | LINC02182 |
| chr16 | 88678113 | 88678864 | 2.18378E−05 | 0.267031694 | SNAI3 |
| chr16 | 88779290 | 88779904 | 1.31589E−07 | 0.273308903 | PIEZO1 |
| chr16 | 89052399 | 89052667 | 8.59398E−13 | −0.490601761 | ACSF3 |
| chr16 | 89576146 | 89576892 | 9.2458E−17 | 0.427291729 | CPNE7 |
| chr17 | 886479 | 886515 | 4.16061E−15 | 0.557803717 | NXN |
| chr17 | 901012 | 901443 | 2.51486E−14 | 0.438301492 | NXN |
| chr17 | 1233283 | 1233390 | 2.63577E−10 | −0.447106294 | ABR |
| chr17 | 1921239 | 1921342 | 1.16909E−06 | −0.323477548 | RTN4RL1 |
| chr17 | 2071724 | 2072256 | 4.16061E−15 | 0.513821243 | SMG6 |
| chr17 | 2174665 | 2174994 | 9.2458E−17 | 0.433770188 | SMG6 |
| chr17 | 2181522 | 2182043 | 8.96843E−15 | 0.529372065 | SMG6 |
| chr17 | 2386295 | 2386656 | 2.27256E−07 | −0.300311835 | MNT |
| chr17 | 3005038 | 3005190 | 2.27256E−07 | −0.328487058 | RAP1GAP2 |
| chr17 | 4391731 | 4391846 | 1.04116E−09 | −0.369655384 | UBE2G1 |
| chr17 | 4542852 | 4543146 | 5.58249E−09 | −0.359275215 | MYBBP1A |
| chr17 | 4900343 | 4901199 | 0.012461837 | 0.152709105 | CHRNE |
| chr17 | 6509500 | 6509771 | 8.29042E−09 | −0.343766502 | PITPNM3 |
| chr17 | 7383732 | 7384717 | 1.90143E−10 | 0.382921837 | TNK1 |
| chr17 | 7588952 | 7589316 | 4.04042E−12 | 0.43380532 | MPDU1 |
| chr17 | 8316052 | 8316129 | 1.36237E−12 | 0.423267376 | ARHGEF15 |
| chr17 | 9206432 | 9207129 | 9.10841E−12 | 0.463269989 | NTN1 |
| chr17 | 16420120 | 16420168 | 7.48307E−08 | −0.297458749 | TRPV2 |
| chr17 | 17506402 | 17506579 | 6.00881E−05 | −0.251088599 | PEMT |
| chr17 | 17711796 | 17711946 | 4.10611E−05 | −0.218937998 | RAI1 |
| chr17 | 17774399 | 17774651 | 1.16909E−06 | 0.222961215 | RAI1 |
| chr17 | 18476730 | 18477149 | 1.17733E−10 | −0.353944001 | LGALS9C |
| chr17 | 21397419 | 21397748 | 2.51486E−14 | 0.426279212 | KCNJ12 |
| chr17 | 27349642 | 27349950 | 4.20778E−10 | −0.346660214 | WSB1 |
| chr17 | 29572822 | 29572891 | 1.85622E−09 | 0.47349715 | TP53I13 |
| chr17 | 29785994 | 29786100 | 4.89446E−11 | −0.37914072 | SSH2 |
| chr17 | 33252355 | 33252513 | 3.24158E−13 | −0.384941449 | ASIC2 |
| chr17 | 35345693 | 35345954 | 2.85148E−11 | 0.390241791 | SLFN11 |
| chr17 | 35488795 | 35488943 | 4.69687E−14 | 0.409058842 | SLFN12L |
| chr17 | 35489861 | 35490166 | 1.9296E−13 | 0.451455685 | SLFN12L |
| chr17 | 35495504 | 35495795 | 2.82847E−09 | 0.369132922 | SLFN12L |
| chr17 | 42458291 | 42458430 | 1.03925E−06 | −0.349139648 | ATP6V0A1 |
| chr17 | 44212679 | 44212809 | 4.97202E−12 | 0.391064189 | UBTF |
| chr17 | 44749405 | 44750345 | 1.70548E−12 | 0.447761679 | DBF4B |
| chr17 | 47861080 | 47861272 | 8.08825E−05 | −0.246970337 | MIR4315-1 |
| chr17 | 48489738 | 48490094 | 1.36869E−10 | −0.395592802 | MIR4315-1 |
| chr17 | 48621599 | 48621944 | 7.46442E−12 | 0.399837226 | HOXB9 |
| chr17 | 49210568 | 49211208 | 1.88966E−06 | 0.209594254 | ABI3 |
| chr17 | 50274167 | 50274335 | 4.16061E−15 | −0.434127306 | TMEM92 |
| chr17 | 55320553 | 55320728 | 1.15832E−10 | −0.459998405 | HLF |
| chr17 | 57519041 | 57519629 | 2.46079E−09 | −0.368651512 | MSI2 |
| chr17 | 58199551 | 58199722 | 6.78457E−13 | −0.39883101 | EPX |
| chr17 | 59782923 | 59783053 | 4.88309E−09 | −0.391610843 | VMP1 |
| chr17 | 59842398 | 59842534 | 3.68705E−07 | −0.397400164 | MIR4315-1 |
| chr17 | 60421358 | 60421861 | 0.012461837 | 0.268962362 | USP32 |
| chr17 | 60578812 | 60579160 | 8.96843E−15 | 0.452893306 | LINC01999 |
| chr17 | 62675338 | 62675526 | 0.004993664 | 0.198623963 | MRC2 |
| chr17 | 62680499 | 62681115 | 0.000100652 | −0.187161674 | MRC2 |
| chr17 | 63435209 | 63435668 | 9.10841E−12 | −0.396367086 | CYB561 |
| chr17 | 63436042 | 63436517 | 4.97202E−12 | −0.428113184 | CYB561 |
| chr17 | 63447194 | 63447458 | 2.77374E−15 | −0.422084345 | MIR4315-1 |
| chr17 | 63698181 | 63698569 | 5.82391E−07 | −0.326607012 | STRADA |
| chr17 | 64701046 | 64701196 | 1.70548E−12 | −0.445294335 | MIR4315-1 |
| chr17 | 65460676 | 65460816 | 3.44869E−14 | −0.420926688 | LINC02563 |
| chr17 | 67025252 | 67025660 | 8.59398E−13 | −0.400888135 | CACNG4 |
| chr17 | 68021277 | 68021668 | 4.16061E−15 | −0.458534795 | KPNA2 |
| chr17 | 70539894 | 70540061 | 9.43988E−09 | −0.386584376 | KCNJ2 |
| chr17 | 72589120 | 72589250 | 5.32836E−13 | −0.388042662 | LINC00511 |
| chr17 | 73366104 | 73366398 | 1.47656E−13 | 0.278742314 | SDK2 |
| chr17 | 73395162 | 73395513 | 2.12644E−12 | −0.435706304 | SDK2 |
| chr17 | 74759499 | 74760221 | 2.50931E−13 | −0.42353684 | SLC9A3R1 |
| chr17 | 75707219 | 75707411 | 1.3454E−11 | −0.389116424 | SAP30BP |
| chr17 | 75753505 | 75754322 | 8.45991E−14 | 0.432492833 | ITGB4 |
| chr17 | 75827932 | 75828091 | 4.16801E−13 | 0.507874005 | UNC13D |
| chr17 | 75835425 | 75835542 | 9.78564E−11 | 0.374550579 | UNC13D |
| chr17 | 76700722 | 76700829 | 4.69687E−14 | 0.527175524 | MXRA7 |
| chr17 | 77526356 | 77526479 | 1.2884E−06 | −0.298235876 | LOC400622 |
| chr17 | 77527052 | 77527620 | 4.89446E−11 | −0.451697642 | LOC400622 |
| chr17 | 77591373 | 77591554 | 4.16061E−15 | −0.467484189 | LOC100507351 |
| chr17 | 77865457 | 77865590 | 3.24748E−09 | 0.421858845 | LINC01973 |
| chr17 | 78132400 | 78132662 | 2.51486E−14 | −0.403737944 | TMC8 |
| chr17 | 78358247 | 78358815 | 1.15832E−10 | −0.430664735 | SOCS3 |
| chr17 | 78358405 | 78358582 | 8.25256E−11 | −0.476499726 | SOCS3 |
| chr17 | 78365093 | 78365324 | 1.61457E−10 | −0.480642334 | SOCS3-DT |
| chr17 | 78526812 | 78527106 | 2.13847E−09 | −0.394260787 | DNAH17 |
| chr17 | 78921642 | 78921879 | 2.51486E−14 | 0.4152105 | TIMP2 |
| chr17 | 78978998 | 78979130 | 2.64282E−12 | −0.447052392 | LGALS3BP |
| chr17 | 79934053 | 79934185 | 8.45991E−14 | −0.496590634 | TBC1D16 |
| chr17 | 80023721 | 80024064 | 2.81406E−07 | 0.353416609 | TBC1D16 |
| chr17 | 80834382 | 80834561 | 1.3454E−11 | 0.441567141 | RPTOR |
| chr17 | 80898744 | 80898939 | 4.17463E−08 | −0.279053309 | RPTOR |
| chr17 | 81037168 | 81037591 | 1.10836E−11 | −0.381732952 | BAIAP2 |
| chr17 | 81071700 | 81072328 | 9.10841E−12 | −0.421282759 | BAIAP2 |
| chr17 | 81202257 | 81202559 | 4.20778E−10 | −0.373850105 | CEP131 |
| chr17 | 81285012 | 81285296 | 6.3755E−09 | 0.321290828 | SLC38A10 |
| chr17 | 81403930 | 81404070 | 2.82847E−09 | 0.397851583 | BAHCC1 |
| chr17 | 81426826 | 81427384 | 2.51486E−14 | −0.467899106 | BAHCC1 |
| chr17 | 81461183 | 81462109 | 2.62456E−10 | −0.433718148 | BAHCC1 |
| chr17 | 81659401 | 81659942 | 2.51486E−14 | −0.428973618 | PDE6G |
| chr17 | 82000478 | 82001107 | 1.04116E−09 | −0.394868437 | ASPSCR1 |
| chr17 | 82830323 | 82830502 | 1.3454E−11 | 0.448714594 | ZNF750 |
| chr17 | 83111254 | 83112201 | 2.82847E−09 | −0.414096764 | METRNL |
| chr17 | 83200018 | 83200276 | 5.57287E−05 | −0.228601167 | RPL23AP87 |
| chr18 | 3014032 | 3014164 | 1.9296E−13 | 0.506840268 | LPIN2 |
| chr18 | 3499026 | 3499863 | 1.62897E−11 | 0.464098723 | DLGAP1 |
| chr18 | 3580208 | 3580944 | 3.4212E−11 | −0.457530608 | DLGAP1 |
| chr18 | 4825987 | 4826188 | 1.70548E−12 | −0.445964141 | LINC01892 |
| chr18 | 11802813 | 11802912 | 3.4212E−11 | −0.425715038 | GNAL |
| chr18 | 12923541 | 12923853 | 4.89446E−11 | −0.404103302 | PTPN2 |
| chr18 | 23331187 | 23331273 | 3.72444E−09 | −0.413288325 | TMEM241 |
| chr18 | 23872965 | 23873168 | 2.51486E−14 | −0.465730972 | LAMA3 |
| chr18 | 24492554 | 24493036 | 1.36237E−12 | −0.44102763 | HRH4 |
| chr18 | 26696391 | 26696550 | 6.19469E−15 | 0.429832672 | PCAT18 |
| chr18 | 37243361 | 37243974 | 6.78457E−13 | 0.441090791 | CELF4 |
| chr18 | 48878654 | 48879220 | 1.80293E−14 | −0.431594011 | CTIF |
| chr18 | 49497049 | 49497189 | 6.47206E−16 | −0.409378838 | RPL17 |
| chr18 | 57428322 | 57428985 | 1.36869E−10 | 0.336637545 | ONECUT2 |
| chr18 | 72866933 | 72866953 | 2.34899E−09 | 0.40546078 | NETO1 |
| chr18 | 74524677 | 74524747 | 4.91037E−10 | −0.416497107 | CNDP2 |
| chr18 | 79775705 | 79776125 | 3.44869E−14 | −0.416633322 | CTDP1 |
| chr18 | 79783162 | 79783363 | 2.64282E−12 | −0.439856489 | CTDP1 |
| chr18 | 79784267 | 79785073 | 1.04116E−09 | −0.360759795 | CTDP1 |
| chr19 | 752158 | 752670 | 1.47656E−13 | −0.447788854 | MISP |
| chr19 | 900866 | 901098 | 0.000154226 | −0.275082263 | R3HDM4 |
| chr19 | 955772 | 956447 | 3.70579E−08 | −0.348143663 | ARID3A |
| chr19 | 956447 | 956669 | 1.41907E−06 | −0.304166259 | ARID3A |
| chr19 | 1074374 | 1075144 | 1.7567E−15 | −0.406687337 | ARHGAP45 |
| chr19 | 1312825 | 1313289 | 2.27256E−07 | −0.298092954 | EFNA2 |
| chr19 | 1371048 | 1371093 | 5.57287E−05 | −0.275922788 | PWWP3A |
| chr19 | 1496436 | 1496615 | 2.64282E−12 | −0.388048799 | REEP6 |
| chr19 | 1528081 | 1528420 | 1.3454E−11 | −0.376772901 | PLK5 |
| chr19 | 1897015 | 1897172 | 1.39359E−09 | −0.399326177 | SCAMP4 |
| chr19 | 2091085 | 2091341 | 9.78564E−11 | 0.379234753 | MOB3A |
| chr19 | 2525679 | 2525903 | 4.97202E−12 | −0.451393997 | GNG7 |
| chr19 | 3098559 | 3098726 | 1.90143E−10 | 0.377321708 | GNA11 |
| chr19 | 3375261 | 3376009 | 4.26687E−09 | 0.32396571 | NFIC |
| chr19 | 3408002 | 3408243 | 4.04042E−12 | 0.375988485 | NFIC |
| chr19 | 3423731 | 3423846 | 6.78457E−13 | 0.442172751 | NFIC |
| chr19 | 3466579 | 3466786 | 3.44869E−14 | 0.430014659 | NFIC |
| chr19 | 3576838 | 3577254 | 1.62897E−11 | 0.443651833 | HMG20B |
| chr19 | 3649855 | 3650110 | 7.87923E−07 | −0.311631142 | PIP5K1C |
| chr19 | 5068530 | 5068724 | 6.00881E−05 | 0.323673467 | KDM4B |
| chr19 | 5298180 | 5298662 | 2.50931E−13 | −0.469689756 | PTPRS |
| chr19 | 5914439 | 5914959 | 2.13847E−09 | −0.359178789 | CAPS |
| chr19 | 5947508 | 5947784 | 6.00881E−05 | 0.280171194 | RANBP3 |
| chr19 | 6259597 | 6259737 | 1.47656E−13 | 0.47410589 | MLLT1 |
| chr19 | 6477106 | 6477219 | 1.36237E−12 | 0.381104337 | DENND1C |
| chr19 | 6753287 | 6753628 | 3.69832E−16 | 0.49210848 | SH2D3A |
| chr19 | 6866150 | 6866261 | 9.78564E−11 | −0.415380304 | VAV1 |
| chr19 | 7254756 | 7255079 | 5.32836E−13 | −0.433910461 | INSR |
| chr19 | 7619320 | 7620313 | 1.3454E−11 | −0.416564088 | XAB2 |
| chr19 | 8655522 | 8655636 | 1.7567E−15 | −0.424632655 | NFILZ |
| chr19 | 9018389 | 9018552 | 3.6005E−10 | −0.432689574 | MUC16 |
| chr19 | 10353230 | 10353581 | 6.78457E−13 | 0.434009276 | TYK2 |
| chr19 | 10817258 | 10818206 | 5.32836E−13 | 0.471028839 | MIR199A1 |
| chr19 | 11165188 | 11165395 | 1.28517E−14 | 0.574664944 | KANK2 |
| chr19 | 11194219 | 11194615 | 1.70548E−12 | 0.399880293 | KANK2 |
| chr19 | 11739704 | 11740129 | 6.9467E−11 | −0.445323891 | ZNF823 |
| chr19 | 12555452 | 12555723 | 6.9467E−11 | 0.409362701 | ZNF564 |
| chr19 | 13207210 | 13207603 | 1.17715E−07 | −0.312459877 | CACNA1A |
| chr19 | 13730780 | 13731071 | 9.78564E−11 | −0.421330239 | YJU2B |
| chr19 | 14417343 | 14418099 | 4.04042E−12 | −0.387242227 | DDX39A |
| chr19 | 15130390 | 15130559 | 9.2458E−17 | −0.397828726 | ILVBL |
| chr19 | 15251717 | 15251746 | 1.25727E−07 | 0.241188203 | BRD4 |
| chr19 | 17415491 | 17415705 | 3.21678E−10 | 0.517438831 | MVB12A |
| chr19 | 17415491 | 17415705 | 3.21678E−10 | 0.517438831 | MVB12A |
| chr19 | 17791367 | 17791441 | 3.4212E−11 | −0.402877411 | FCHO1 |
| chr19 | 18155937 | 18156133 | 1.04116E−09 | −0.38121547 | PIK3R2 |
| chr19 | 18443079 | 18443277 | 8.98223E−10 | 0.250322949 | ELL |
| chr19 | 18649789 | 18650310 | 1.84916E−16 | 0.500241077 | KLHL26 |
| chr19 | 18650936 | 18651116 | 2.37133E−11 | 0.432745761 | KLHL26 |
| chr19 | 18711326 | 18711687 | 1.90143E−10 | 0.413966391 | CRTC1 |
| chr19 | 18726013 | 18726297 | 2.77374E−15 | 0.406538696 | CRTC1 |
| chr19 | 18764112 | 18764581 | 2.74694E−06 | −0.334368598 | CRTC1 |
| chr19 | 18848929 | 18849313 | 4.16061E−15 | 0.474888322 | UPF1 |
| chr19 | 18870380 | 18870414 | 1.84916E−16 | 0.469202102 | GDF1 |
| chr19 | 18952738 | 18952869 | 1.38562E−08 | 0.395045526 | HOMER3-AS1 |
| chr19 | 19378241 | 19378755 | 2.51486E−14 | −0.406531792 | GATAD2A |
| chr19 | 19514169 | 19514289 | 9.2458E−17 | 0.483417818 | TSSK6 |
| chr19 | 19514315 | 19514696 | 9.2458E−17 | 0.538472939 | TSSK6 |
| chr19 | 19629001 | 19629218 | 1.28517E−14 | −0.39834999 | LPAR2 |
| chr19 | 19637506 | 19637567 | 1.7567E−15 | 0.381656121 | GMIP |
| chr19 | 20424219 | 20424775 | 4.16061E−15 | 0.494440839 | ZNF826P |
| chr19 | 22519763 | 22519777 | 2.13847E−09 | 0.350940923 | LOC105376917 |
| chr19 | 29636892 | 29637180 | 1.84916E−16 | 0.440012335 | POP4 |
| chr19 | 29636896 | 29637180 | 3.69832E−16 | 0.444684841 | POP4 |
| chr19 | 30670476 | 30670787 | 5.32836E−13 | −0.411886213 | ZNF536 |
| chr19 | 31517594 | 31517705 | 2.12644E−12 | −0.453927163 | THEG5 |
| chr19 | 31761267 | 31761631 | 6.19469E−15 | −0.395043916 | THEG5 |
| chr19 | 31883344 | 31883735 | 3.69832E−16 | −0.539142968 | LINC01533 |
| chr19 | 31943430 | 31943604 | 2.82847E−09 | −0.385680257 | LINC01533 |
| chr19 | 33058085 | 33058258 | 1.60932E−09 | −0.420774518 | RHPN2 |
| chr19 | 33903266 | 33903340 | 3.27265E−12 | −0.442039288 | KCTD15 |
| chr19 | 35126608 | 35126920 | 4.7463E−07 | 0.303563631 | LGI4 |
| chr19 | 36132318 | 36132637 | 1.28517E−14 | −0.445233118 | TBCB |
| chr19 | 36151715 | 36152426 | 6.47206E−16 | 0.586928637 | COX7A1 |
| chr19 | 37932697 | 37932961 | 1.08444E−12 | −0.477656673 | SIPA1L3 |
| chr19 | 38183419 | 38183926 | 1.08444E−12 | 0.502338654 | SIPA1L3 |
| chr19 | 38266299 | 38266561 | 7.46442E−12 | −0.483873455 | SPINT2 |
| chr19 | 38304257 | 38304462 | 1.47656E−13 | −0.397592046 | YIF1B |
| chr19 | 38415037 | 38415079 | 1.36237E−12 | −0.465317373 | RASGRP4 |
| chr19 | 38661812 | 38661948 | 9.43988E−09 | −0.398772363 | ACTN4 |
| chr19 | 39308383 | 39308435 | 8.29042E−09 | 0.327023598 | LRFN1 |
| chr19 | 39431030 | 39431216 | 8.96843E−15 | −0.418914663 | RPS16 |
| chr19 | 40609451 | 40609892 | 6.47206E−16 | −0.438719447 | LTBP4 |
| chr19 | 43773920 | 43775048 | 4.69687E−14 | 0.437853463 | KCNN4 |
| chr19 | 43783128 | 43783258 | 4.97202E−12 | −0.401247409 | KCNN4 |
| chr19 | 45038391 | 45038619 | 3.4212E−11 | −0.389033737 | RELB |
| chr19 | 45783169 | 45783489 | 1.15832E−10 | 0.394661877 | DMWD |
| chr19 | 46217546 | 46217650 | 1.80293E−14 | −0.427376685 | LOC93429 |
| chr19 | 46697529 | 46697779 | 1.17715E−07 | 0.389706952 | PRKD2 |
| chr19 | 47424964 | 47425190 | 3.44869E−14 | −0.402801068 | SLC8A2 |
| chr19 | 47718427 | 47718690 | 1.80293E−14 | 0.415313014 | EHD2 |
| chr19 | 47782065 | 47782207 | 9.2458E−17 | 0.573723145 | SELENOW |
| chr19 | 48615068 | 48615135 | 7.46442E−12 | −0.385054769 | RPL18 |
| chr19 | 48927416 | 48927732 | 4.89446E−11 | −0.426062496 | NUCB1 |
| chr19 | 49494671 | 49494734 | 1.08444E−12 | −0.470519931 | RPL13A |
| chr19 | 49887510 | 49888134 | 2.64282E−12 | −0.373104973 | TBC1D17 |
| chr19 | 50163606 | 50163742 | 1.1095E−15 | −0.421137825 | IZUMO2 |
| chr19 | 50517130 | 50517378 | 3.27265E−12 | −0.429836368 | LRRC4B |
| chr19 | 52584933 | 52585494 | 1.61457E−10 | 0.342177317 | ZNF701 |
| chr19 | 52943603 | 52943705 | 1.16909E−06 | −0.361941856 | ZNF816-ZNF321P |
| chr19 | 53851118 | 53851317 | 6.78457E−13 | −0.464772805 | MYADM |
| chr19 | 55086208 | 55087009 | 3.4212E−11 | 0.406294623 | EPS8L1 |
| chr19 | 55118861 | 55118979 | 1.28517E−14 | 0.392616731 | PPP1R12C |
| chr19 | 55645033 | 55645169 | 8.98223E−10 | −0.412799586 | ZNF581 |
| chr19 | 56105852 | 56105886 | 1.61733E−07 | −0.395760661 | ZNF787 |
| chr19 | 56234366 | 56234698 | 8.38991E−08 | −0.365903437 | ZSCAN5A |
| chr19 | 56840344 | 56840644 | 0.404752974 | 0.136440543 | PEG3 |
| chr19 | 58204157 | 58204852 | 8.59398E−13 | 0.500993087 | ZNF274 |
| chr20 | 279256 | 279565 | 1.90143E−10 | 0.431795603 | C20orf96 |
| chr20 | 419367 | 419628 | 2.46079E−09 | −0.389645188 | RBCK1 |
| chr20 | 856470 | 856645 | 1.47656E−13 | −0.478633525 | FAM110A |
| chr20 | 2289349 | 2289542 | 3.72444E−09 | −0.408536173 | TGM3 |
| chr20 | 2809202 | 2809528 | 1.36869E−10 | −0.38670432 | TMEM239 |
| chr20 | 3221437 | 3221845 | 3.72444E−09 | −0.385772539 | ITPA |
| chr20 | 4161941 | 4162363 | 4.16061E−15 | −0.461040057 | SMOX |
| chr20 | 5112007 | 5112155 | 2.12644E−12 | −0.438596776 | TMEM230 |
| chr20 | 5126089 | 5126128 | 1.7567E−15 | −0.483825839 | PCNA |
| chr20 | 5452392 | 5452552 | 9.2458E−17 | −0.463599954 | LOC643406 |
| chr20 | 10623295 | 10623551 | 1.96776E−11 | 0.485460102 | SLX4IP |
| chr20 | 10623295 | 10623551 | 1.96776E−11 | 0.485460102 | SLX4IP |
| chr20 | 10667076 | 10667296 | 0.000100652 | −0.251883232 | JAG1 |
| chr20 | 11406645 | 11407108 | 4.91037E−10 | −0.425597496 | LOC339593 |
| chr20 | 16670420 | 16670739 | 1.3454E−11 | −0.444888029 | SNRPB2 |
| chr20 | 17870313 | 17870483 | 2.71981E−10 | 0.417879439 | SNX5 |
| chr20 | 21396147 | 21396878 | 3.70579E−08 | 0.264040522 | NKX2-4 |
| chr20 | 21608428 | 21608677 | 1.28517E−14 | −0.445732015 | LINC01727 |
| chr20 | 22576581 | 22576937 | 2.50931E−13 | 0.440605577 | LINC00261 |
| chr20 | 22581840 | 22582635 | 4.20778E−10 | 0.412191095 | FOXA2 |
| chr20 | 24745837 | 24746706 | 8.96843E−15 | −0.393835554 | SYNDIG1 |
| chr20 | 24778704 | 24778989 | 4.97202E−12 | −0.421782448 | SYNDIG1 |
| chr20 | 24930595 | 24931006 | 6.19469E−15 | −0.482821995 | CST7 |
| chr20 | 24949318 | 24949513 | 1.36237E−12 | −0.38685599 | CST7 |
| chr20 | 25105347 | 25105730 | 6.32413E−14 | −0.417371874 | VSX1 |
| chr20 | 31721823 | 31722030 | 1.47656E−13 | −0.434712167 | BCL2L1 |
| chr20 | 33651776 | 33652011 | 1.61457E−10 | −0.39207935 | CBFA2T2 |
| chr20 | 33733310 | 33733528 | 4.91037E−10 | −0.485525703 | ZNF341 |
| chr20 | 34089654 | 34089878 | 1.60932E−09 | −0.379265875 | EIF2S2 |
| chr20 | 35434413 | 35434650 | 4.16061E−15 | 0.445801506 | GDF5 |
| chr20 | 40887332 | 40887453 | 4.97202E−12 | −0.453334633 | TOP1 |
| chr20 | 44651190 | 44651230 | 9.60906E−07 | −0.306800911 | ADA |
| chr20 | 45973340 | 45973788 | 1.7567E−15 | −0.433613205 | ZNF335 |
| chr20 | 46251122 | 46251649 | 2.12644E−12 | 0.426819434 | CDH22 |
| chr20 | 49567524 | 49567657 | 4.20778E−10 | 0.38005467 | PTGIS |
| chr20 | 50079084 | 50079642 | 1.1095E−15 | −0.450493294 | UBE2V1 |
| chr20 | 50086933 | 50087063 | 1.3454E−11 | −0.417272275 | UBE2V1 |
| chr20 | 50086933 | 50087288 | 1.3454E−11 | −0.420102215 | UBE2V1 |
| chr20 | 50237304 | 50237666 | 9.78564E−11 | −0.41788212 | PELATON |
| chr20 | 50243264 | 50243305 | 1.15832E−10 | −0.389046007 | PELATON |
| chr20 | 50305358 | 50305548 | 1.1095E−15 | 0.502016605 | LINC01270 |
| chr20 | 50741701 | 50741842 | 1.61457E−10 | −0.418139429 | PARD6B |
| chr20 | 51766763 | 51766968 | 6.19469E−15 | −0.490244611 | ATP9A |
| chr20 | 51985642 | 51985712 | 2.77374E−15 | −0.540215251 | ZFP64 |
| chr20 | 53521387 | 53521739 | 9.2458E−17 | −0.476316303 | TSHZ2 |
| chr20 | 54123835 | 54124057 | 8.45991E−14 | −0.508331507 | CYP24A1 |
| chr20 | 56625229 | 56625855 | 6.47206E−16 | 0.506545242 | TFAP2C |
| chr20 | 56627381 | 56627452 | 1.10343E−07 | 0.331802488 | TFAP2C |
| chr20 | 56627831 | 56627872 | 9.2458E−17 | 0.470940326 | TFAP2C |
| chr20 | 57708084 | 57708221 | 6.4762E−05 | 0.286759771 | PMEPA1 |
| chr20 | 57711947 | 57712175 | 6.32413E−14 | −0.439455139 | NKILA |
| chr20 | 57947898 | 57948123 | 9.2458E−17 | 0.413106661 | LINC01742 |
| chr20 | 58017823 | 58017973 | 6.19469E−15 | −0.503869522 | LINC01742 |
| chr20 | 58604339 | 58604756 | 1.47656E−13 | 0.440755712 | APCDD1L-DT |
| chr20 | 58980682 | 58980842 | 3.44869E−14 | −0.485120413 | NELFCD |
| chr20 | 61490422 | 61490623 | 7.73912E−10 | −0.415778813 | CDH4 |
| chr20 | 61532733 | 61532941 | 1.9296E−13 | −0.415776432 | CDH4 |
| chr20 | 61558836 | 61559041 | 1.47656E−13 | −0.442140232 | CDH4 |
| chr20 | 61651473 | 61651534 | 1.12059E−13 | −0.432083252 | CDH4 |
| chr20 | 62350383 | 62351095 | 4.04042E−12 | −0.399310812 | LAMA5 |
| chr20 | 62356318 | 62356417 | 7.78903E−10 | 0.413409878 | LAMA5 |
| chr20 | 62426452 | 62426887 | 6.47206E−16 | −0.419388328 | RBBP8NL |
| chr20 | 62427448 | 62427811 | 2.50931E−13 | −0.478654806 | RBBP8NL |
| chr20 | 62647888 | 62648289 | 1.2053E−09 | 0.334089829 | SLCO4A1 |
| chr20 | 63369986 | 63370155 | 1.62897E−11 | −0.419527004 | CHRNA4 |
| chr20 | 63479783 | 63480139 | 1.7567E−15 | −0.42011736 | KCNQ2 |
| chr20 | 63506269 | 63506566 | 5.32836E−13 | −0.496134099 | EEF1A2 |
| chr20 | 63537304 | 63537577 | 9.43988E−09 | −0.38519228 | PTK6 |
| chr20 | 63689585 | 63689975 | 2.64282E−12 | −0.451584499 | RTEL1 |
| chr20 | 63775324 | 63775545 | 1.28517E−14 | 0.445276069 | ZBTB46 |
| chr20 | 63791820 | 63792124 | 3.69832E−16 | 0.499198431 | ZBTB46 |
| chr21 | 14077469 | 14077907 | 1.78134E−08 | −0.4338127 | CBS |
| chr21 | 21257675 | 21257876 | 4.69895E−08 | −0.3270904 | CBS |
| chr21 | 32499348 | 32499847 | 1.28517E−14 | 0.443328059 | CBS |
| chr21 | 32572889 | 32573077 | 1.90143E−10 | −0.400529489 | CBS |
| chr21 | 33541236 | 33541304 | 1.61457E−10 | −0.444053711 | CBS |
| chr21 | 36547528 | 36547746 | 0.001702963 | −0.207928089 | CBS |
| chr21 | 38980963 | 38981471 | 1.12059E−13 | −0.411523202 | CBS |
| chr21 | 39519268 | 39519495 | 1.96776E−11 | −0.410293538 | CBS |
| chr21 | 41387390 | 41387418 | 1.39831E−05 | 0.266562642 | CBS |
| chr21 | 41681080 | 41681668 | 1.10836E−11 | 0.408859726 | CBS |
| chr21 | 41725676 | 41725985 | 8.98223E−10 | 0.373605925 | CBS |
| chr21 | 42741525 | 42741775 | 9.2458E−17 | 0.500113179 | CBS |
| chr21 | 42955445 | 42955867 | 1.08444E−12 | −0.448833701 | CBS |
| chr21 | 43001681 | 43001817 | 9.2458E−17 | 0.536977343 | CBS |
| chr21 | 43860193 | 43860572 | 6.9467E−11 | 0.385060142 | AGPAT3 |
| chr21 | 44380262 | 44380350 | 2.51486E−14 | 0.427011622 | TRPM2 |
| chr21 | 44958599 | 44959139 | 2.62456E−10 | 0.362644766 | FAM207A |
| chr21 | 45258214 | 45258521 | 1.36237E−12 | −0.390301186 | LINC00334 |
| chr21 | 45513443 | 45513645 | 1.96776E−11 | −0.388465211 | COL18A1 |
| chr21 | 45950246 | 45950479 | 3.4212E−11 | −0.418989049 | PCBP3 |
| chr21 | 46110570 | 46110791 | 4.91037E−10 | 0.416792429 | COL6A2 |
| chr22 | 17366280 | 17366482 | 3.69832E−16 | −0.451451576 | CECR2 |
| chr22 | 17808854 | 17809014 | 0.000133983 | −0.259370088 | MICAL3 |
| chr22 | 19869351 | 19869480 | 1.10836E−11 | −0.433663722 | TXNRD2 |
| chr22 | 20241823 | 20242384 | 3.70579E−08 | −0.384949902 | RTN4R |
| chr22 | 20249698 | 20249873 | 4.16061E−15 | 0.55245222 | RTN4R |
| chr22 | 20280279 | 20280407 | 9.78564E−11 | −0.42243315 | RTN4R |
| chr22 | 23099125 | 23099181 | 1.88966E−06 | 0.330658025 | GNAZ |
| chr22 | 23157004 | 23157399 | 1.08444E−12 | −0.458776315 | RAB36 |
| chr22 | 23181938 | 23182209 | 4.73786E−06 | −0.317972943 | BCR |
| chr22 | 23237047 | 23237104 | 4.32317E−09 | 0.430143955 | BCR |
| chr22 | 23237706 | 23237764 | 1.9296E−13 | 0.477221528 | BCR |
| chr22 | 28801556 | 28801748 | 5.72228E−10 | −0.394099651 | XBP1 |
| chr22 | 29329014 | 29329165 | 1.04116E−09 | 0.450245556 | AP1B1 |
| chr22 | 30080077 | 30080444 | 1.84916E−16 | 0.418256089 | HORMAD2-AS1 |
| chr22 | 30210497 | 30210852 | 4.16801E−13 | −0.451789963 | LIF-AS1 |
| chr22 | 31039613 | 31039815 | 3.24158E−13 | 0.462126574 | SMTN |
| chr22 | 31344124 | 31345076 | 2.77374E−15 | −0.4115879 | PATZ1 |
| chr22 | 35301441 | 35301654 | 2.46079E−09 | −0.438728982 | TOM1 |
| chr22 | 36435835 | 36436080 | 1.04116E−09 | −0.400144457 | TXN2 |
| chr22 | 36459201 | 36459766 | 2.50931E−13 | −0.379270429 | TXN2 |
| chr22 | 37626358 | 37626701 | 1.36237E−12 | 0.415046901 | GGA1 |
| chr22 | 38214298 | 38214499 | 2.91289E−08 | 0.30024333 | PLA2G6 |
| chr22 | 39292218 | 39292554 | 6.19469E−15 | −0.410383931 | RPL3 |
| chr22 | 40652462 | 40653123 | 8.96843E−15 | −0.448005141 | MRTFA |
| chr22 | 42289889 | 42290089 | 7.27381E−09 | 0.419133666 | TCF20 |
| chr22 | 43297629 | 43298056 | 1.28517E−14 | 0.37678979 | SCUBE1 |
| chr22 | 44340669 | 44340782 | 2.62456E−10 | −0.385733457 | SHISAL1 |
| chr22 | 44639081 | 44639367 | 0.033119501 | 0.164797854 | LINC00229 |
| chr22 | 44729504 | 44730253 | 1.9296E−13 | 0.403440892 | PRR5 |
| chr22 | 45125079 | 45125252 | 8.98223E−10 | 0.38024475 | NUP50-DT |
| chr22 | 45919419 | 45919668 | 4.04042E−12 | −0.443062546 | WNT7B |
| chr22 | 46090364 | 46090437 | 1.36237E−12 | 0.372850584 | MIRLET7BHG |
| chr22 | 46374548 | 46375406 | 6.78457E−13 | −0.440368161 | CELSR1 |
| chr22 | 46443966 | 46444207 | 1.15832E−10 | −0.372478312 | CELSR1 |
| chr22 | 46532876 | 46533865 | 4.04042E−12 | −0.403980108 | CELSR1 |
| chr22 | 46640828 | 46641112 | 1.15832E−10 | 0.404559506 | GRAMD4 |
| chr22 | 49949273 | 49949926 | 1.08444E−12 | −0.407230518 | PIM3 |
| chr22 | 50282728 | 50282923 | 1.96776E−11 | −0.397606316 | PLXNB2 |
| chr22 | 50548243 | 50549095 | 1.15832E−10 | 0.351844091 | KLHDC7B |
| chr22 | 50603903 | 50604331 | 6.4762E−05 | 0.247597835 | MAPK8IP2 |

In some embodiments, the target genomic regions that are examined to differentiate epithelial ovarian cancer from a benign tumor in a subject comprise at least 5%, at least 10%, at least 15%, at least 20%, at least 25%, at least 30%, at least 40%, at least 45%, at least 50%, at least 55%, at least 60%, at least 65%, at least 70%, at least 75%, at least 80%, a least 85%, at least 90%, at least 95%, at least 96%, at least 97%, at least 98%, at least 99%, or 100% of the target genomic regions listed in Table 1.

In some embodiments, the target genomic regions that are examined to differentiate high grade serous epithelial ovarian cancer from non-high grade serous epithelial ovarian cancer in a subject comprise at least 5%, at least 10%, at least 15%, at least 20%, at least 25%, at least 30%, at least 40%, at least 45%, at least 50%, at least 55%, at least 60%, at least 65%, at least 70%, at least 75%, at least 80%, a least 85%, at least 90%, at least 95%, at least 96%, at least 97%, at least 98%, at least 99%, or 100% of the target genomic regions listed in Table 1.

In some embodiments, a method for detecting high grade serous epithelial ovarian cancer in a subject comprising, consisting essentially of, or consisting of the steps of (a) measuring the level of nucleic acid methylation of a plurality of target genomic region listed in Table 1 from a cell-free nucleic acid sample from the subject; (b) comparing the level of nucleic acid methylation of the plurality of target genomic region in the sample to the level of nucleic acid methylation of the plurality of target genomic regions in a sample isolated from a cancer-free subject, a cancer-free reference standard, or a cancer-free reference cutoff value; (c) determining that the subject has high grade serous epithelial ovarian cancer based on a change in the level of nucleic acid methylation in the plurality of target genomic regions in the sample derived from the subject, wherein the change is greater or lower than the level of nucleic acid methylation of the target genomic regions in the sample isolated from a cancer-free subject, a normal reference standard, or a normal reference cutoff value.

In some embodiments, a method for differentiating high grade serous epithelial ovarian cancer from non-high grade serous epithelial cancer in a subject a method for detecting high grade serous epithelial ovarian cancer in a subject comprising, consisting essentially of, or consisting of the steps of (a) measuring the level of nucleic acid methylation of a plurality of target genomic region listed in Table 1 from a cell-free nucleic acid sample from the subject; (b) comparing the level of nucleic acid methylation of the plurality of target genomic region in the sample to the level of nucleic acid methylation of the plurality of target genomic regions in a sample isolated from a cancer-free subject, a cancer-free reference standard, or a cancer-free reference cutoff value; (c) determining that the subject has high grade serous epithelial ovarian cancer based on a change in the level of nucleic acid methylation in the plurality of target genomic regions in the sample derived from the subject, wherein the change is greater or lower than the level of nucleic acid methylation of the target genomic regions in the sample isolated from a non-high grade serous epithelial ovarian cancer subject.

In some embodiments, the target genomic regions that are examined to determine the presence or absence of ovarian cancer, the severity of ovarian cancer, the histological subtype of ovarian cancer, and other methods described herein in a subject comprise at least 5%, at least 10%, at least 15%, at least 20%, at least 25%, at least 30%, at least 40%, at least 45%, at least 50%, at least 55%, at least 60%, at least 65%, at least 70%, at least 75%, at least 80%, a least 85%, at least 90%, at least 95%, at least 96%, at least 97%, at least 98%, at least 99%, or 100% of the target genomic regions listed in Table 1 but exclude the genomic sequences of Table 2.

TABLE 2
Target genomic regions excluded in some embodiments. The
target genomic regions may be found in the known human
reference genome hg38, which is available from Genome
Refence Consortium with a reference number GRCh38/hg38.

| Chromosome | Start | Stop |
|---|---|---|
| Chr2 | 38323997 | 38324203 |
| Chr2 | 113712408 | 113712611 |
| Chr3 | 20029245 | 20029704 |
| Chr8 | 58146211 | 58146673 |
| Chr8 | 124995553 | 124995624 |
| Chr9 | 89438825 | 89439085 |
| Chr11 | 63664463 | 63664769 |
| Chr11 | 120496972 | 120497256 |
| Chr20 | 5452392 | 5452552 |

In some embodiments, sequencing of the target region is achieved by next-generation sequencing. In some embodiments, the next-generation sequencing comprises one or more of pyrosequencing, single-molecule real-time sequencing, sequencing by synthesis, sequencing by ligation (SOLID sequencing), or nanopore sequencing.

In some embodiments, the detection of cfDNA in the sample further comprises aligning the DNA sequences from the next-generation sequencing to a human reference genome. In a specific embodiment, the human reference genome GRCh38 (UCSC version hg38) and is incorporated herein in its entirety.

In some embodiments, the nucleotide sequences that are examined for nucleic acid methylation levels include the target genomic region sequences listed in Table 1 and also may include the immediately adjacent 1-100, 1-150, 1-200, 1-300, 1-400, 1-500, 500-1000, 1000-1500, 1500-2000, 2000-2500, 2500-3000, 3000-3500, or 3500-4000 nucleotides upstream or downstream of a target genomic region listed in Table 1.

In some embodiments, the level of nucleic acid methylation is determined at a genomic region within the selected gene or genes. Non-limiting examples include a genomic region within an untranslated region (UTR) of the selected gene or genes, a genomic region within 1.5 kb upstream of the transcription start site of the selected gene or genes, and a genomic region within the first exon of the selected gene or genes.

In some embodiments of the methods described herein, the DNA methylation levels of the target genomic regions disclosed in Table 1 are compared to the methylation levels of the same target genomic regions of a control sample or standard (a known non-cancerous sample). In some embodiments, the control samples are known non-cancerous cells and/or known cancerous cells from patients or pools of patients. In some embodiments, the difference in a methylation level of a target genomic region that is indicative of cancer compared to the methylation level of the same gene region from a control sample or reference standard is about 0.2 to about 0.65 (see Table 1, column labeled “dmr value”). A probability score based on the totality differences in nucleic acid methylation of each target genomic region compared to a control target genomic region can determine the presence or absence of ovarian cancer, and/or the stage of ovarian cancer, type of ovarian cancer, susceptibility to ovarian cancer, etc.

Embodiments of the methods described herein also may be used to determine the methylation level of certain target genomic regions that are implicated in various tumors to predict, for example, malignancy or stages of malignancy. Exemplary tumors include leukemias, including acute leukemias (such as 11q23-positive acute leukemia, acute lymphocytic leukemia, acute myelocytic leukemia, acute myelogenous leukemia and myeloblastic, promyelocytic, myelomonocytic, monocytic and erythroleukemia), chronic leukemias (such as chronic myelocytic (granulocytic) leukemia, chronic myelogenous leukemia, and chronic lymphocytic leukemia), polycythemia vera, lymphoma, Hodgkin's disease, non-Hodgkin's lymphoma (indolent and high grade forms), multiple myeloma, Waldenstrom's macroglobulinemia, heavy chain disease, myelodysplastic syndrome, hairy cell leukemia and myelodysplasia. Other tumors may include sarcomas and carcinomas, include fibrosarcoma, myxosarcoma, liposarcoma, chondrosarcoma, osteogenic sarcoma, and other sarcomas, synovioma, mesothelioma, Ewing's tumor, leiomyosarcoma, rhabdomyosarcoma, colon carcinoma, lymphoid malignancy, pancreatic cancer, breast cancer (including basal breast carcinoma, ductal carcinoma and lobular breast carcinoma), lung cancers, ovarian cancer, prostate cancer, hepatocellular carcinoma, squamous cell carcinoma, basal cell carcinoma, adenocarcinoma, sweat gland carcinoma, medullary thyroid carcinoma, papillary thyroid carcinoma, pheochromocytomas sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, seminoma, bladder carcinoma, and CNS tumors (such as a glioma, astrocytoma, medulloblastoma, craniopharyrgioma, ependymoma, pinealoma, hemangioblastoma, acoustic neuroma, oligodendroglioma, meningioma, melanoma, neuroblastoma and retinoblastoma).

Using, for example, the target genomic regions listed in Table 1, embodiments of the invention can have greater than 75% sensitivity in detecting early to late stage cancer ovarian cancer, greater than 80% sensitivity in detecting early to late stage ovarian cancer, greater than 85% sensitivity in detecting early to late stage ovarian cancer, greater than 90% sensitivity in detecting early to late stage ovarian cancer, greater than 95% sensitivity in detecting early to late stage ovarian cancer, greater than 96% sensitivity in detecting early to late stage ovarian cancer, greater than 97% sensitivity in detecting early to late stage ovarian cancer, greater than 98% sensitivity in detecting early to late stage ovarian cancer, greater than 99% sensitivity in detecting early to late stage ovarian cancer, or 100% sensitivity in detecting early to late stage ovarian cancer. Embodiments of the invention also may have greater than 50% specificity in detecting early to late stage ovarian cancer, greater than 60% specificity in detecting early to late stage ovarian cancer, greater than 70% specificity in detecting early to late stage ovarian cancer, greater than 75% specificity in detecting early to late stage ovarian cancer, greater than 80% specificity in detecting early to late stage ovarian cancer, greater than 85% specificity in detecting early to late stage ovarian cancer, greater than 90% specificity in detecting early to late stage ovarian cancer, or greater than 95% specificity in detecting early to late stage ovarian cancer.

Upon identifying a subject as likely to develop cancer or cancer recurrence (e.g., a type of ovarian cancer), a prophylactic procedure or therapy can be administered to the subject. For example, prophylactic measures include but are not limited to surgery, tamoxifen administration, and raloxifene administration. For solid tumors, surgical resection can be performed.

Upon identifying a subject as having ovarian cancer or ovarian cancer recurrence, a clinical procedure or cancer therapy can be administered to the subject. For ovarian cancer, exemplary therapies or procedures include but are not limited to surgery, radiation therapy, chemotherapy, hormone therapy, targeted therapy, and/or administration of one or more of: Abitrexate (Methotrexate), Abraxane (Paclitaxel Albumin-stabilized Nanoparticle Formulation), Ado-Trastuzumab Emtansine, Afinitor (Everolimus), Anastrozole, Aredia (Pamidronate Disodium), Arimidex (Anastrozole), Aromasin (Exemestane), Capecitabine, Clafen, (Cyclophosphamide), Cyclophosphamide, Cytoxan (Cyclophosphamide), Docetaxel, Doxorubicin Hydrochloride, Ellence (Epirubicin Hydrochloride), Epirubicin Hydrochloride, Eribulin Mesylate, Everolimus, Exemestane, 5-FU (Fluorouracil Injection), Fareston (Toremifene), Faslodex (Fulvestrant), Femara (Letrozole), Fluorouracil Injection, Folex (Methotrexate), Folex PFS (Methotrexate), Fulvestrant, Gemcitabine Hydrochloride, Gemzar (Gemcitabine Hydrochloride), Goserelin Acetate, Halaven (Eribulin Mesylate), Herceptin (Trastuzumab), Ibrance (Palbociclib), Ixabepilone, Ixempra (Ixabepilone), Kadcyla (Ado-Trastuzumab Emtansine), Kisqali (Ribociclib), Lapatinib Ditosylate, Letrozole, Megestrol Acetate, Methotrexate, Methotrexate LPF (Methotrexate), Mexate (Methotrexate), Mexate-AQ (Methotrexate), Neosar (Cyclophosphamide), Neratinib Maleate, Nerlynx (Neratinib Maleate), Nolvadex (Tamoxifen Citrate), Paclitaxel, Paclitaxel Albumin-stabilized Nanoparticle Formulation, Palbociclib, Pamidronate Disodium, Perjeta (Pertuzumab), Pertuzumab, Ribociclib, Tamoxifen Citrate, Taxol (Paclitaxel), Taxotere (Docetaxel), Thiotepa, Toremifene, Trastuzumab, Tykerb (Lapatinib Ditosylate), Velban (Vinblastine Sulfate), Velsar (Vinblastine Sulfate), Vinblastine Sulfate, Xeloda (Capecitabine), and Zoladex (Goserelin Acetate).

In one embodiment, the method for treating cancer may include administering a pharmaceutical composition that includes a pharmaceutically acceptable carrier and a therapeutically effective amount of a compound listed above that inhibits the genes or protein products of the gene associated with the target genomic regions listed in Table 1.

In some embodiments, method of treatment of a cancer may include a suitable substance able to target intracellular proteins, small molecules, or nucleic acid molecules alone or in combination with an appropriate carrier or vehicle, including, but not limited to, an antibody or functional fragment thereof, (e.g., Fab′, F(ab′)2, Fab, Fv, rlgG, and scFv fragments and genetically engineered or otherwise modified forms of immunoglobulins such as intrabodies and chimeric antibodies), small molecule inhibitors of the protein, chimeric proteins or peptides, gene therapy for inhibition of transcription, or an RNA interference (RNAi)-related molecule or morpholino molecule able to inhibit gene expression and/or translation. In one embodiment the inhibitor is an RNAi-related molecule such as an siRNA or an shRNA for inhibition of translation. An RNA interference (RNAi) molecule is a small nucleic acid molecule, such as a short interfering RNA (siRNA), a double-stranded RNA (dsRNA), a micro-RNA (miRNA), or a short hairpin RNA (shRNA) molecule, that complementarily binds to a portion of a target gene or mRNA so as to provide for decreased levels of expression of the target.

Various aspects of the methods disclosed herein (e.g., for identifying a benign or malignant tumor or mass in a subject) can be implemented using computer-based calculations, machine learning (e.g., support vector machine (SVM), Lasso and Elastic-Net Regularized Generalized Linear Models (Glmnet), Random Forest, Gradient boosting (on random forest), C5.0 decision trees), and other software tools. For example, a methylation status for a CpG site can be assigned by a computer based on an underlying sequence read of an amplicon from a sequencing assay. In another example, a methylation value for a DNA region or portion thereof can be compared by a computer to a threshold value, as described herein. The tools are advantageously provided in the form of computer programs that are executable by a general-purpose computer system of conventional design.

In some embodiments, the method used to analyze and/or determine methylation levels of a target polynucleotide region includes Metilene (Juhling et al., Genome Res., 2016; 26(2): 256-262) or GenomeStudio Software available online from Illumina, Inc., or as described in Hovestadt et al., 2014; Nature, 510(7506), 537-541.

In some embodiments, methods of identifying ovarian cancer or a severity thereof in a subject may comprise the use of a machine learning algorithm. The machine learning algorithm may be a trained algorithm. The machine learning algorithm may be trained on one or more features and trained be used to process a data set generated via assaying nucleic acid molecules in a sample (e.g., cell-free biological sample), which data set comprises a methylation profile of one or more genomic regions of the cell-free biological sample.

The machine learning algorithm (e.g., trained machine learning algorithm) may be configured to identify a presence of epithelial ovarian cancer at an accuracy of at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99%.

Target genomic regions may be identified (e.g., using the methods provided herein) to have differential methylation in samples from subjects having ovarian cancer as compared to samples from subjects not having ovarian cancer. In other embodiments, the methylation level or one or more target regions may be associated with a first stage of ovarian cancer but may not be associated with a second stage of ovarian cancer. In another example, the methylation level or one or more target regions may not be associated with a first stage of ovarian cancer but may be associated with a second stage of ovarian cancer. The methylation levels of other target regions may be associated with the second stage of ovarian cancer and may or may not also be associated with the first stage.

In some embodiments, the nucleic acid molecules may be contacted with an array of probes under conditions to allow hybridization. The degree of hybridization of the probes to the nucleic acid molecules may be assayed in a quantitative matter using a number of methods. The degree of hybridization at a probe position may be related to the intensity of signal provided by the assay, which therefore is related to the amount of complementary nucleic acid sequence present in the sample. Software can be used to extract, normalize, summarize, and analyze array intensity data from probes across the human genome or transcriptome including expressed genes, exons, introns, and miRNAs. The intensity of a given probe in either the cancerous or non-cancerous samples may be compared against a reference set to determine whether differential methylation is occurring in a sample. An increase or decrease in relative intensity at a marker position on an array corresponding to an expressed sequence may be indicative of an increase or decrease respectively of methylation of the corresponding marker or gene. Sequencing assays may also be used to determine amounts or relative amounts of specific nucleic acid sequences (e.g., nucleic acid sequences of nucleic acid molecules of a sample, such as a cell-free biological sample). Such nucleic acid sequences may include nucleic acid sequences associated with specific genomic regions of interest (e.g., genomic regions comprising genes and/or markers). Sequencing data may be processed to assign values (e.g., intensity values) to given nucleic acid sequences or features thereof (e.g., sequences associated with differentially methylated regions).

Values (e.g., intensity values) associated with given nucleic acid sequences for a sample can be analyzed using feature selection techniques including filter techniques which assess the relevance of features by looking at the intrinsic properties of the data, wrapper methods which embed the model hypothesis within a feature subset search, and embedded techniques in which the search for an optimal set of features is built into a classifier algorithm. Filter techniques may include parametric methods such as the use of two sample t-tests, ANOVA analyses, Bayesian frameworks, Gamma distribution models, and non-parametric methods such as, but not limited to, Mann Whitney U test; model free methods such as the use of Wilcoxon rank sum tests, between—within class sum of squares tests, rank products methods, or random permutation methods; and multivariate methods such as bivariate methods, correlation based feature selection methods (CFS), minimum redundancy maximum relevance methods (MRMR), Markov blanket filter methods, and uncorrelated shrunken centroid methods. Wrapper methods may include sequential search methods, genetic algorithms, and estimation of distribution algorithms. Embedded methods may include random forest algorithms, weight vector of support vector machine algorithms, and weights of logistic regression algorithms.

Selected features may be classified using a classifier algorithm. Illustrative algorithms include methods that reduce the number of variables such as principal component analysis algorithms, partial least squares methods, and independent component analysis algorithms. Illustrative algorithms may handle large numbers of variables directly such as statistical methods and methods based on machine learning techniques. Statistical methods include penalized logistic regression, prediction analysis of microarrays (PAM), methods based on shrunken centroids, support vector machine analysis, and regularized linear discriminant analysis.

A trained machine learning algorithm may comprise a supervised machine learning algorithm. The trained machine learning algorithm may comprise a classification and regression tree (CART) algorithm. The supervised machine learning algorithm may comprise, for example, a Random Forest, a support vector machine (SVM), a neural network, a deep learning algorithm, a bagging procedure, or a boosting procedure. The trained machine learning algorithm may comprise an unsupervised machine learning algorithm. The trained machine learning algorithm may be configured to accept a plurality of input variables and to produce one or more output values based on the plurality of input variables. The plurality of input variables may comprise methylation profiles of one or more genomic regions of one or more cell-free biological samples.

The trained machine learning algorithm may comprise a classifier, such that each of the one or more output values comprises one of a fixed number of possible values (e.g., a linear classifier, a logistic regression classifier, etc.) indicating a classification of the cell-free biological sample by the classifier. The trained machine learning algorithm may comprise a binary classifier, such that each of the one or more output values comprises one of two values (e.g., (0, 1}, (positive, negative}, (positive for ovarian cancer, negative for ovarian cancer}indicating a classification of the cell-free biological sample by the classifier. The trained machine learning algorithm may be another type of classifier, such that each of the one or more output values comprises one of more than two values (e.g., (0, 1, 21 or (positive, negative, or indeterminate}) indicating a classification of the cell-free biological sample by the classifier. The output values may comprise descriptive labels, numerical values, or a combination thereof. Some descriptive labels may be mapped to numerical values, for example, by mapping “positive” to 1 and “negative” to 0.

Some of the output values may comprise numerical values, such as binary, integer, or continuous values. Such binary output values may comprise, for example, (0, 1}. Such integer output values may comprise, for example, (0, 1, 2}. Such continuous output values may comprise, for example, a probability value of at least 0 and no more than 1. Such continuous output values may comprise, for example, an un-normalized probability value of at least 0. Such continuous output values may comprise, for example, an un-normalized probability value of at least 0. Such continuous output values may indicate a presence, severity, and/or prognosis of an ovarian cancer of the subject. Such continuous output values may indicate a prediction of the therapeutic regimen to treat the ovarian cancer of the subject and may comprise, for example, an indication of an expected duration of efficacy of the therapeutic regimen. Some numerical values may be mapped to descriptive labels, for example, by mapping 1 to “positive” and 0 to “negative”.

Some of the output values may be assigned based on one or more cutoff values. For example, a binary classification of samples may assign an output value of “positive” or 1 if the sample indicates that the subject has at least a 50% probability of having ovarian cancer. For example, a binary classification of samples may assign an output value of “negative” or 0 if the sample indicates that the subject has less than a 50% probability of having ovarian cancer. In this case, a single cutoff value of 50% is used to classify samples into one of the two possible binary output values. Examples of single cutoff values may include about 1%, 2%, 5%, 10%, 15%, 20%, 25%, 30%, 35%, 40%, 45%, 50%, 55%, 60%, 65%, 70%, 75%, 80%, 85%, 90%, 95%, 98%, and 99%. For example, the single cutoff value may be between about 1% and about 99%, such as between about 10% and about 90%, such as between about 10% and about 75%, such as between about 10% and about 60%, about 10% and about 50%, about 20% and about 75%, about 20% and about 60%, about 20% and about 50%, about 30% and about 75%, about 30% and about 60%, about 30% and about 50%, 40% and about 75%, 40% and about 60%, 40% and about 50%, 50% and about 75%, or about 50% and about 60%.

The trained machine learning algorithm may be trained with a plurality of independent training samples. Each of the independent training samples may comprise a biological sample (e.g., cell-free biological sample) from a subject, and/or associated data obtained by processing the biological sample (as described elsewhere herein), and/or one or more known output values corresponding to the biological sample (e.g., a clinical diagnosis, prognosis, treatment efficacy, or a presence, absence, or severity of a ovarian cancer of the subject). Independent training samples may comprise biological samples (e.g., cell-free biological samples) and/or associated data and outputs obtained from a plurality of different subjects. Independent training samples may comprise biological samples (e.g., cell-free biological samples) and associated data and outputs obtained at a plurality of different time points from the same subject (e.g., before, after, and/or during a course of treatment to treat ovarian cancer of the subject). Independent training samples may be associated with a presence or severity of the ovarian cancer (e.g., training samples comprising cell-free biological samples and associated data and outputs obtained from a plurality of subjects known to have ovarian cancer and/or various stages of ovarian cancer (e.g., stage I epithelial ovarian cancer, stage II epithelial ovarian cancer, stage III epithelial ovarian cancer, and stage IV epithelial ovarian cancer). This also may include any histological subtype of epithelial ovarian cancer such, but not limited to endometrioid ovarian cancer, mucinous ovarian cancer, clear cell ovarian cancer, and serous ovarian cancer and various stages of each histological subtype of epithelial ovarian cancer. Independent training samples may be associated with an absence of ovarian cancer (e.g., training samples comprising cell-free biological samples and associated data and outputs obtained from a plurality of subjects who are known to not have a previous diagnosis of ovarian cancer, who have recovered from ovarian cancer, or who are otherwise asymptomatic for ovarian cancer). In other embodiments, independent training sample may be associated with high grade serous epithelial ovarian cancer. In other embodiments, training samples may be associated with non-high grade epithelial ovarian cancer.

The trained machine algorithm may be trained with at least about 20, at least about 30, at least about 40, at least about 50, at least about 60, at least about 70, at least about 80, at least about 90, at least about 100, at least about 150, at least about 200, at least about 250, at least about 300, at least about 350, at least about 400, at least about 450, at least about 500, or more independent training samples.

The trained machine learning algorithm may be trained with tissue samples (e.g., tumorous samples or non-tumorous samples), cell-free samples (e.g., cell-free nucleic acid samples), or a combination thereof.

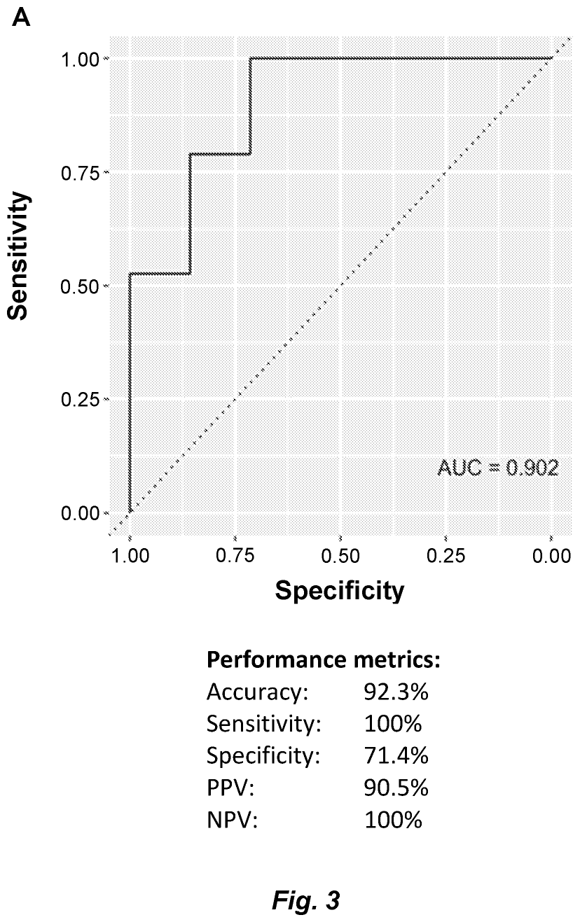

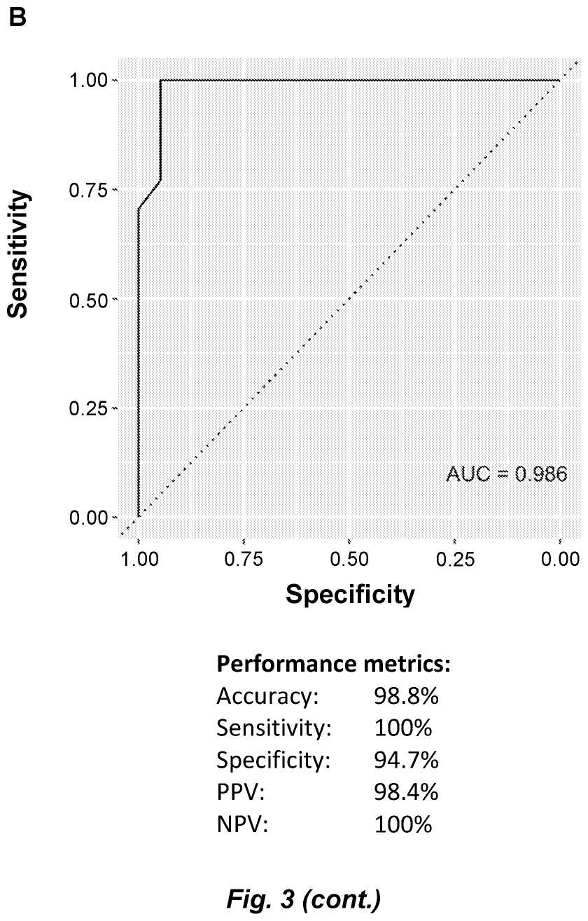

In some embodiments, the machine learning algorithm may be trained using a plurality of cell-free nucleic acid collected from subjects having cancer free/normal ovaries and/or fallopian tubes in which the methylation levels of the target genomic regions of Table 1 are compared to the methylation of the same target genomic regions of Table 1 from cell-free nucleic acids obtained from a subject having an epithelial ovarian cancer. Subject derived biological samples (e.g., cell-free DNA samples) are then examined for methylation levels of the target genomic regions of Table 1. The trained machine learning algorithm then outputs a probability value based on the differentially methylated regions of Table I that the subject derived biological sample is, for example, cancerous or the severity of the cancer. A user may set a threshold probability value that is indicative of the condition based on the strongest separation of the conditions (see for example, FIG. 3a).

In other embodiments, the machine learning algorithm may be trained using a plurality of nucleic acid samples collected from cancer free/normal ovaries and/or fallopian tube tissue samples in which the methylation levels of the target genomic regions of Table 1 are compared to the methylation of the same target genomic regions of Table 1 from tissue of known tumorous tissue (e.g., known ovarian cancer tissue samples). Once trained, the machine learning algorithm may be used to analyze target genomic regions of Table 1 in a subject to determine the presence of absence, or the severity of ovarian cancer in the subject. In some embodiments, the machine learning algorithm, once trained on using a plurality of nucleic acid samples collected from cancer free/normal ovaries and/or fallopian tube tissue samples in which the methylation levels of the target genomic regions of Table 1 are compared to the methylation of the same target genomic regions of Table 1 from tissue of known tumorous tissue, may be used as the trained machine algorithm to determine, for example, the presence or absence of epithelial ovarian cancer, the severity of epithelial ovarian cancer, the histological subtype of epithelial ovarian cancer, the susceptibility to epithelial ovarian cancer, differentiate between high grade serous epithelial ovarian cancer and non-high grade serous epithelial ovarian cancer, differentiate between a benign tumor and epithelial ovarian cancer, and indicate the presence of an epithelial ovarian cancer in an asymptomatic subject or in a subject genetically predisposed to a type of cancer

In some embodiments, a differential methylation value (DMV) of about 10, about 15, about 18, about 20, about 22, about 25, about 30, about 35, about 40, about 45, about 50, about 55, or about 60 (in percent scale) is considered a differentially methylated locus (DML) or differentially methylated region (DMR). In some embodiments, a DMV of about 20 percent is considered a DML or DMR. In some embodiments, a P value less than about 0.05 is considered a DML or DMR.

In some embodiments, a subject may be determined to have or develop cancer or cancer recurrence if DNA methylation is enriched at the selected genomic target regions as compared to the normal control sample, the reference standard, or the cutoff value. In some embodiments, the reference cutoff value is a DMV of about 10, about 15, about 18, about 20, about 22, about 25, about 30, about 35, about 40, about 45, about 50, about 55, or about 60 (in percent scale). In some embodiments, the reference cutoff value is about 40 percent.

The machine learning algorithm (e.g., trained machine learning algorithm) may be configured to identify a presence or absence of epithelial ovarian cancer, the severity of epithelial ovarian cancer, the histological subtype of epithelial ovarian cancer, the susceptibility to epithelial ovarian cancer, differentiate between high grade serous epithelial ovarian cancer and non-high grade serous epithelial ovarian cancer, differentiate between a benign tumor and epithelial ovarian cancer, and indicate the presence of an epithelial ovarian cancer in an asymptomatic subject or in a subject genetically predisposed to a type of cancer at an accuracy of at least about 50%, at least about 65%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% for at least about 10, 20, 30, 40, 50, 100, 200, 250, 300, 400, 500, or more independent samples. The accuracy of identifying the presence or severity of the ovarian cancer by the trained machine learning algorithm may be calculated as the percentage of independent test samples (e.g., subjects known to have the severity of ovarian cancer or apparently healthy subjects with negative clinical test results for the severity of ovarian cancer) that are correctly identified or classified as having or not having the severity of ovarian cancer.

The machine learning algorithm (e.g., trained machine learning algorithm) may be configured to identify a presence or absence of epithelial ovarian cancer, the severity of epithelial ovarian cancer, the histological subtype of epithelial ovarian cancer, the susceptibility to epithelial ovarian cancer, differentiate between high grade serous epithelial ovarian cancer and non-high grade serous epithelial ovarian cancer, differentiate between a benign tumor and epithelial ovarian cancer, and indicate the presence of an epithelial ovarian cancer in an asymptomatic subject or in a subject genetically predisposed to a type of cancer with an Area-Under-Curve (AUC) of at least about 0.50, at least about 0.55, at least about 0.60, at least about 0.65, at least about 0.70, at least about 0.75, at least about 0.80, at least about 0.81, at least about 0.82, at least about 0.83, at least about 0.84, at least about 0.85, at least about 0.86, at least about 0.87, at least about 0.88, at least about 0.89, at least about 0.90, at least about 0.91, at least about 0.92, at least about 0.93, at least about 0.94, at least about 0.95, at least about 0.96, at least about 0.97, at least about 0.98, at least about 0.99, or higher. The AUC may be calculated as an integral of the Receiver Operator Characteristic (ROC) curve (e.g., the area under the ROC curve) associated with the algorithm in classifying cell-free biological samples as having or not having the severity of the disease.

The methods described herein also may be implemented by use of computer systems. For example, any of the steps described above for evaluating sequence reads to determine methylation status of a CpG site may be performed by means of software components loaded into a computer or other information appliance or digital device. When so enabled, the computer, appliance or device may then perform all or some of the above-described steps to assist the analysis of values associated with the methylation of a one or more CpG sites, or for comparing such associated values. The above features embodied in one or more computer programs may be performed by one or more computers running such programs.

In some embodiments, a computer comprising at least one processor may be configured to receive a plurality of sequencing results from the DNA methylation sequencing reactions that may comprise the methylation level of a region of the one or more genes disclosed herein from a patient having the mass (e.g., pelvic mass) or other tumor and the sequencing results of normal control methylation level of the same genes from the a healthy control sample, compare the plurality of sequencing results from the DNA methylation sequencing comprising the methylation level of the one or more genes disclosed herein from a patient having the mass or other tumor to the normal control methylation level of the one or more genes from the control sample to produce a probability score, and rank a patient based on the probability score. The probability score corresponds to a reference methylation scale such that a low probability score is indicative of a low likelihood of a pelvic mass being cancerous and a high probability score is indicative of high likelihood of a pelvic mass being cancer.

In some embodiments, probability scores are calculated by the machine learning algorithm (e.g., C5.0 decision trees) for each unknown sample based on the machine learning model. The probability score represents the likelihood that the specific sample belongs to an individual with stage I-IV ovarian cancer and not a benign tumor. For, example, a high probability score (>0.45) indicates that the individual is predicted to have a malignant tumor, while low probability score (<0.45) indicates that the individual is predicted to have a benign tumor. In some embodiments, a high probability score (>0.45) indicates that the individual is predicted to have high grade epithelial ovarian cancer, while low probability score (<0.45) indicates that the individual is predicted not to have high grade epithelial ovarian cancer. In some embodiments, a high probability score (>0.45) indicates that the individual is predicted to have epithelial ovarian cancer, while low probability score (<0.45) indicates that the individual is predicted to have a benign tumor. In some embodiments, a high probability score (>0.45) indicates that the individual is predicted to be susceptible to epithelial ovarian cancer, while a low probability score (<0.45) indicates that the individual is predicted not to be susceptible to epithelial ovarian cancer. In some embodiments, a high probability score (<0.45) predicts the presence of an epithelial ovarian cancer in an asymptomatic subject or in a subject genetically predisposed to a type of cancer, while low probability score (<0.45) indicates the absence of an epithelial ovarian cancer in an asymptomatic subject or in a subject genetically predisposed to a type of cancer.

The disclosure provides for methods that permit preoperative determination of whether certain tumors or masses (e.g., a pelvic mass) are benign or malignant, and may be used to discriminate between various stages of cancer progression in a malignant diagnosis. For example, a method for determining preoperatively whether a tumor or other mass is benign or malignant may comprise the steps of a) obtaining a preoperative biological sample from the patient; b) determining a methylation level of one or more target genomic regions from the biological sample; c) comparing the methylation level of the one or more target genomic regions of the biological sample with a methylation level of a normal control methylation level of the one or more target genomic regions obtained from one or more control samples; and d) determining a probability that the pelvic mass from the patient is benign or malignant wherein the probability score of 0.5 or higher based on the methylation levels of the one or more target genomic regions from the biological sample being at least 10% higher or lower compared to the normal control methylation level of the one or more target genomic regions from the one or more control samples indicates malignancy. The one or more target genomic regions are listed in Table 1. When the tumor or mass is determined to be malignant, it may be treated, for example, by radiation therapy, administration of a therapeutic compound (i.e., anti-cancer compound), removal of the tumor or mass from the patient, or a combination thereof.

### Example 1. Development of DNA Methylation Testing Methods

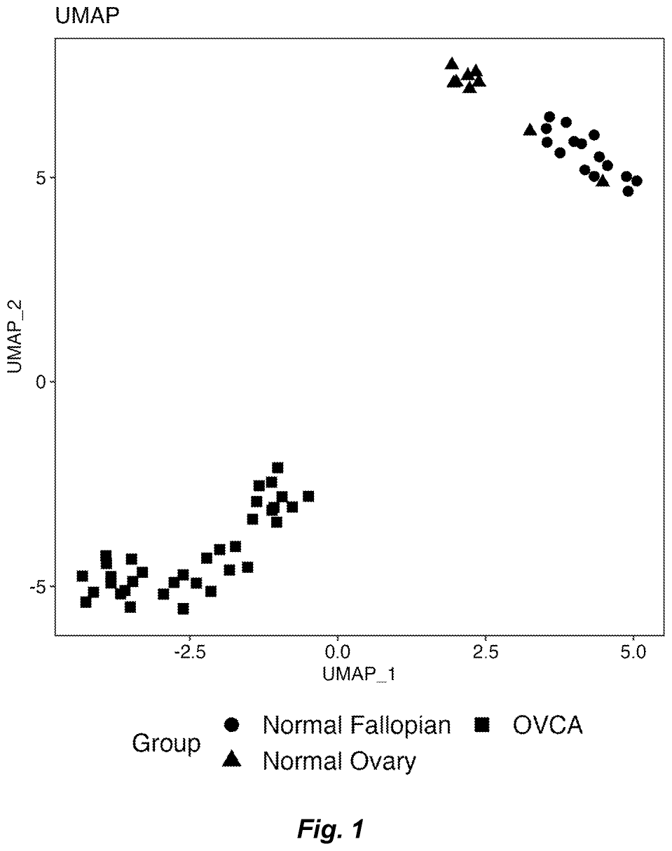

During the discovery phase, 10972 differentially methylated regions (DMRs) were identified between high grade serous epithelial ovarian cancer (HGSOC) and normal fallopian tube samples (FIG. 1). From this data, we selected 35 DMRs for validation using targeted bisulfite amplicon sequencing (bAmplicon-seq) on an independent cohort of plasma-derived cfDNA. This independent validation cohort consisted of benign (n=21), stage I (n=27), stage II (n=3), and stage III (n=31) patient plasma samples.

For biomarker discovery, reduced representation bisulfite sequencing (RRBS) was first performed on tissue from a patient cohort consisting of 33 stage I HGSOC and 10 normal fallopian tube tissue samples from contra-lateral ovaries from patients with EOC. Sequencing libraries were prepared on bisulfite converted DNA and paired-end sequencing performed on an Illumina sequencing platform. Metilene software was used to identify 10972 differentially methylated regions (DMRs) between HGSOC and normal. Unsupervised hierarchical clustering analysis of these regions separated normal samples from HGSOC tumors. From these data, we selected the top 35 DMRs for validation using targeted bisulfite amplicon sequencing (bAmplicon-seq) on an independent cohort of plasma-derived cfDNA. This independent validation cohort consisted of benign (n=21), stage I (n=27), stage II (n=3), and stage III (n=31) patient plasma samples.

Cell-free DNA was bisulfited converted and amplified in a multiplex PCR reaction for the regions of interest. The amplified DNA was then converted into a sequencing library and sequenced using the Illumina MiSeq system. Sequence reads were aligned to the human genome (hg38) using open source Bismark Bisulfite Read Mapper with Bowtie2 alignment algorithm.

In order to construct a novel classifier that can differentiate between patients with HGSOC and those with benign ovarian lesions, we applied machine learning models to the bAmplicon-seq methylation data of the 35 DMRs. Samples were randomly split into a training (70% of samples) set used for generating the model, and a testing (30% of samples) set used to validate the model. Machine learning algorithms constructed a model consisting of the most informative DMRs.

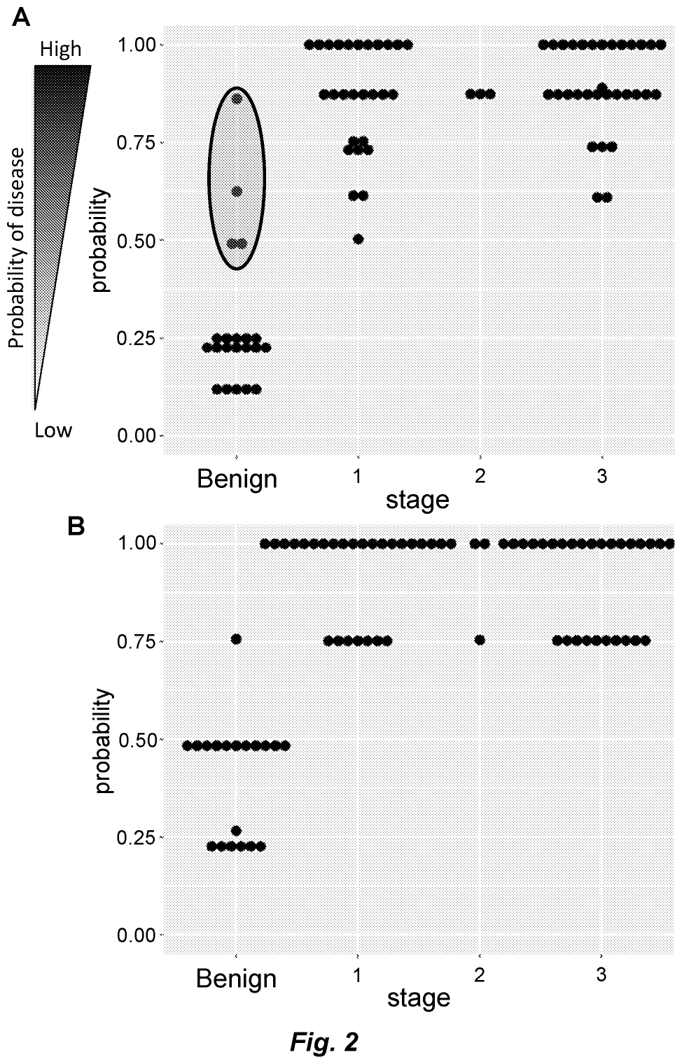

Machine learning algorithms constructed a model consisting of the most informative DMRs. A low score indicates the sample came from a benign pelvic mass, while a high score indicates that the individual has stage I or higher EOC. Although embodiments of the disclosure were derived from stage 1 EOC samples, we found that it was able to stratify benign versus stage I-III EOC (FIG. 2). Furthermore, the ability to identify early stage (stage I) EOC is quite advantageous, since many other EOC diagnostic tests have a lower accuracy in detecting stage I EOC.

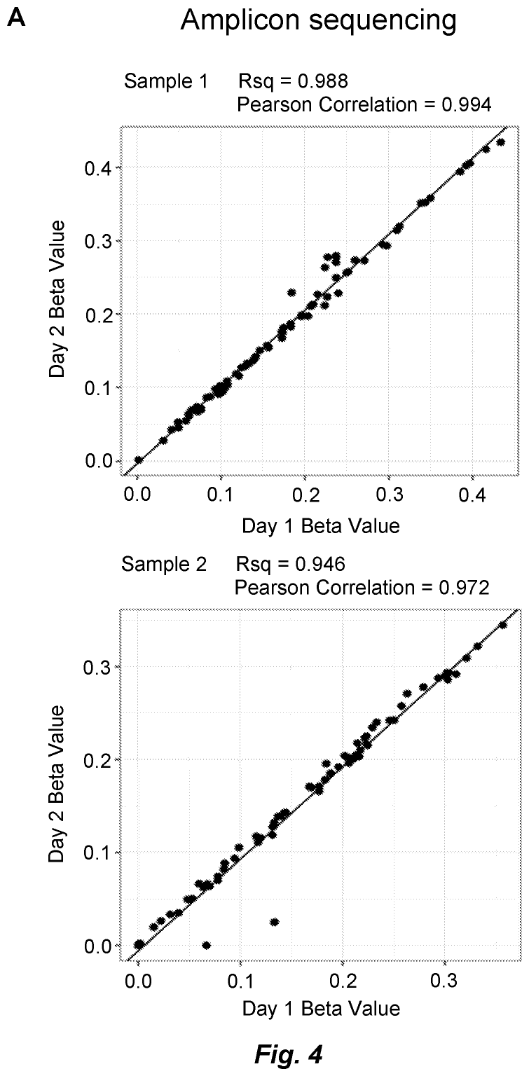

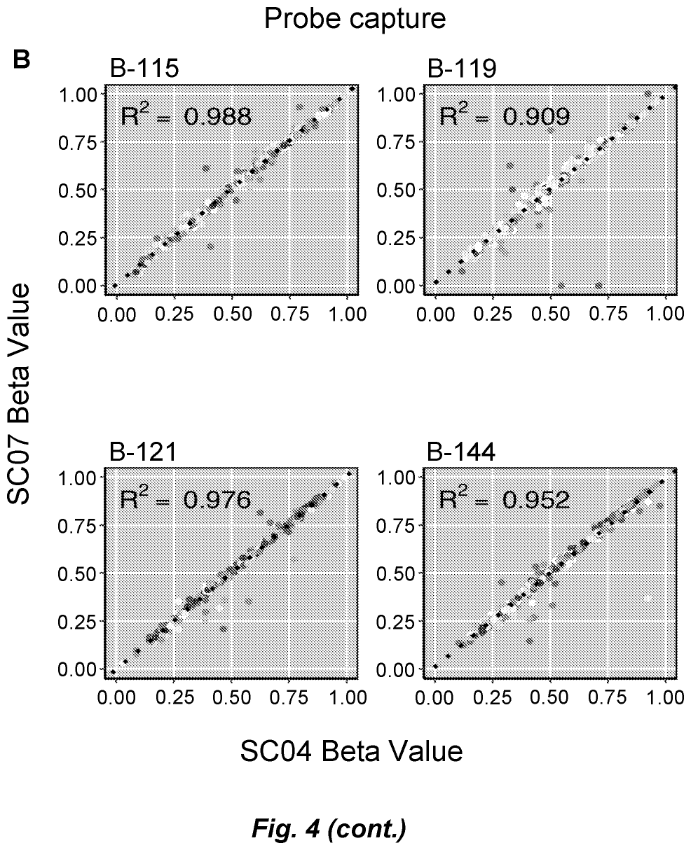

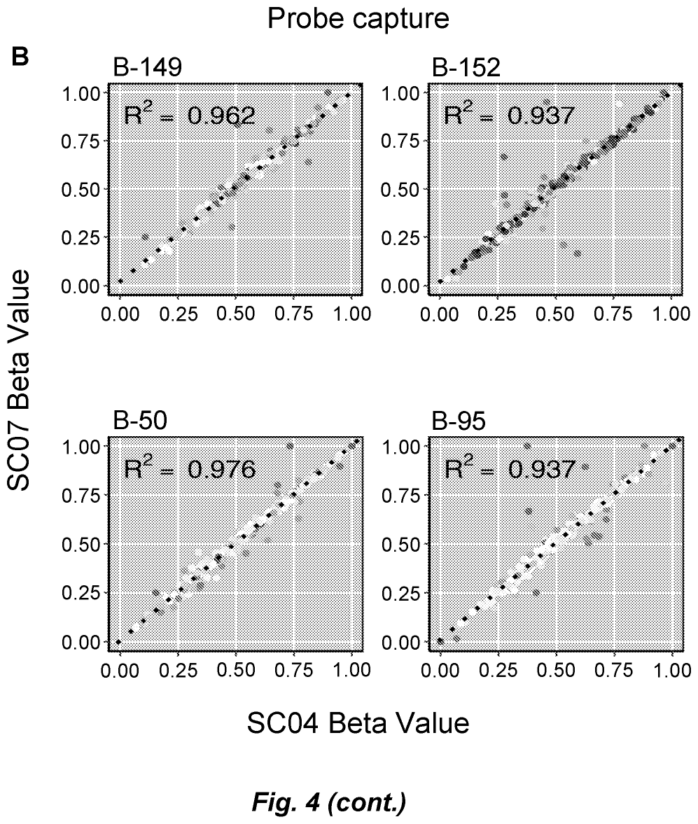

Using the scores obtained from the testing set, we generated a receiver operating characteristic (ROC) curve, which had an area under the curve (AUC) of 0.902 (FIG. 3a). Using the optimal threshold score for classification, the model had high sensitivity (100%) and specificity (71.4%) to diagnose early to late stage EOC. In this instance there were 4 false positive cases but 1 sample was taken from a patient with vulval intraepithelial neoplasia (VIN) and later developed stage 1 clear cell of the vulva 4 years later and another sample was taken from a patient with a history of cervical cancer. After these samples were removed the specificity increased to 94.7% (FIG. 3b). Bisulfite amplicon sequencing and hybrid probe capture are highly reproducible assays. This is evident with the analysis of biological replicates run at different times for bisulfite amplicon sequencing (FIG. 4a) or hybrid probe capture (FIG. 4b). Correlation coefficients (R2) comparing beta values between biological replicates exceeds 0.9 which is indicative of a strong linear relationship and reproducible assay.

In a separate RRBS data analysis, we identified many DMRs between HGSOC and normal fallopian and normal ovarian samples. In this rendition we selected 1677 unique DMRs for further analysis with a hybrid probe capture approach. Hybrid probe capture uses biotinylated RNA probes. To design the probes representing the regions of interest, a variety of CpG methylation states for a given set of targets were synthesized. Probe candidates 60-80 nucleotides in length were then tiled across these targets with 1 probe every 40 nucleotides (˜2× tiling). These were then screened for specificity against both strands of hg38 where all CpH were converted to TpH (i.e., a fully-CpG-methylated genome reference). A final probe set of about 115,739 sequences (93,483 unique) were designed.

Next, cfDNA from a large cohort of plasma samples harvested from patients with benign and malignant adnexal masses was extracted and bisulfite treated. This was followed by library preparation and indexing amplification with unique dual 8 bp indexing primers. Each library was analyzed and quantitated using standard methods. Target enrichment was carried out using a hybrid probe capture design. Bisulfite-converted DNA libraries were incubated with 5′-biotinylated RNA probes and blockers in hybridization buffer overnight. Probe-bounded libraries were pulled down with streptavidin beads followed by washes and an amplifications step. The enriched libraries were quantified and sequenced on a next-generation sequencing platform.

We have developed a laboratory workflow that combines discovery-based genome-wide methylation analysis, target selection, and laboratory validation with clinical validation. Accordingly, the DNA methylation levels of up to 1600 regions in circulation—can be used for the diagnosis of EOC by accurately distinguishing between benign and malignant pelvic masses or can be used to screen asymptomatic women with ovarian cancer.

Various histological subtypes of EOC. Histological subtypes of EOC include endometrioid, mucinous, clear cell and serous. HGSOC are the most common histological subtype and clinically the most aggressive. Here, we perform bAmplicon-seq on 87 non-HGSOC EOC tumors in addition to samples from clinical validation studies to assess the specificity and sensitivity to detect other histological subtypes of EOC. Using these predictions, we compute the AUC and positive/negative predictive value of the assay separately for each histological subtype. We compare the results for each subtype to those for EOC using a two-sample binomial test. This will determine a statistically significant higher or lower sensitivity/specificity for each histological subtype compared to EOC.

Clinical epigenetic subclassification of EOC. Preliminary data show that there may be at least 3 epigenetic subtypes of EOC (FIG. 1) of which the clinical significance is undetermined. To define the relevance of each subtype, we examine clinical correlates such as outcome, BRCA status, age, menopausal status, and relapse. In addition, we determine the importance of co-molecular variates such as mutations and copy number alterations assessed in cfDNA. Lastly, we determine whether these subtypes are related to EOC originating from the fallopian tube or the ovary.

### Example 2. Machine Learning Algorithm

Machine learning model building was performed on DNA methylation data obtained from hybridization-based capture of previously identified differentially methylated regions (DMRs). The methylation values of DMRs were used as the features for model building. Samples and features were initially filtered by sequencing coverage. 5-fold cross validation was performed on the entire sample set, with 20% of the samples used as the test set for each round. Various machine learning models were tested, including random forest, C5.0 decision trees, support vector machine (SVM), generalized linear model (GLM) and gradient boosting. Models were optimized using the area under the curve (AUC) of the receiver operating characteristic (ROC) curve. More advanced models included a feature selection method prior to model construction, such as identification of differential methylation sub-regions. Finalized models are then used to score and classify unknown samples based on the methylation of their DMRs.

### Example 3. Generalizability of Methods Across Different Histological Subtypes of EOC

Preliminary data shows that the target genomic regions described herein are excellent biomarker for HGSOC. EOC includes multiple histologic subtypes such as HGSOC, clear cell, endometrioid and mucinous. HGSOC was chosen for the discovery cohort as this is the most common histologic subtype of ovarian cancer, behaves aggressively and presents at later stages of disease. However, clinically, it would be extremely useful to know if the methods disclosed herein also function for detection of other histologic subtypes of EOC.

Being able to detect EOC of all histologic subtypes would improve the overall outcome for these patients by ensuring they receive the appropriate clinical care. In this aim we plan to test generalizability of OvaPrint™ to the other histologic subtypes of EOC. We have obtained a series of mucinous, clear cell, endometroid and mixed histology HGSOC tumors from GTFR as listed in Table 3. We will perform the testing on these additional 87 tumors by measuring the DNA methylation of each CpG in the selected regions using hybrid capture or with bAmnplicon-seg as described in above.

TABLE 3
Non-HGSOC tumors to be tested

| Histology | # Samples |
|---|---|
| Ovarian mucinous | 30 (Stage I: n = 19) |
| Ovarian clear cell | 18 (Stage I: n = 8) |
| Ovarian endometrioid | 24 (Stage I: n = 19) |
| Ovarian mixed histology | 15 |

We will first determine whether the methylation values for these regions are similar across all histological subtypes, including HGSOC, and if they are distinct from benign samples. We will perform hierarchical clustering and generate a heatmap of methylation values for all samples, including HGSOC and benign samples. Other methods of data clustering, such as multidimensional scaling (MDS) or uniform manifold approximation and projection (UMAP) will also be used. These methods will allow us to assess whether the benign cluster is sufficiently distinct from all histological subtypes, or whether there are specific subtypes that behave more similarly to the benign samples. If a histological subtype forms its own distinct cluster, it suggests that it has its own distinct methylation signature and may not benefit from testing.

Statistical Analysis. In addition to the graphical approaches above, we will formally assess the ability to detect the other EOC subtypes. Methylation values will be entered into the machine learning model, previously built using the HGSOC data, to generate prediction scores for each of the new samples. Using the predictions, we will compute the specificity, sensitivity, and the negative and positive predictive values of the assay separately for each histological subtype. We will formally compare the specificity and the negative predictive value for each subtype to those for HGSOC using a two-sample binomial test to determine a statistically significant higher or lower specificity and sensitivity for each histological subtype compared to HGSOC. Based on these results, we will be able to assess whether the disclosed model generated for HGSOC could be generalized to other histological subtypes. If not, we would refine the model to encompass one or more of the other subtypes or choose to leave them out of the prediction.

### Example 4. Targeted Bisulfite Amplicon Sequencing

Targeted bisulfite amplicon sequencing is performed, for example, on Illumina's MiSeq platform. This nascent, deep-sequencing strategy allows for sensitive detection of DNA methylation in low-input samples such as plasma. Exemplary methods for performing this assay are described in Masser et al. (2015) J Vis Exp. (96): 52488, incorporated herein by reference.

Briefly, nucleic acids are isolated from the sample and quantified. Bisulfite conversion of DNA (e.g., cell-free DNA) is performed using, for example, a commercially available kit such as EZ DNA Methylation™ Kit (available from Zymo Research, Tustin, Calif., USA), EpiMark® Bisulfite Conversion Kit (available from New England Biolabs, Inc., Ipswich, Mass., USA), and Epitect Bisulfite Kits (available from Qiagen, Germantown, Md., USA). Bisulfite conversion changes the unmethylated cytosines into uracils. These uracils are subsequently converted to thymines during later PCR amplification.

Bisulfite converted DNA is amplified by bisulfite specific PCR using a polymerase capable of amplifying bisulfite converted DNA. DNA approximately 60-500 bp in length corresponding to the regions listed in Table 1 are amplified. Amplicons are visualized by PAGE electrophoresis. Alternatively, capillary electrophoresis with a DNA chip is used according to manufacturer's protocol.

A next generation sequencing library is prepared with the amplicons. Nonlimiting examples of methods for preparing the library include using a transposome-mediated protocol with dual indexing, and/or a kit (e.g., TruSeq Methyl Capture EPIC Library Prep Kit, Illumina, CA, USA, Kapa Hyper Prep Kit (Kapa Biosystems). Adapters such as TruSeq DNA LT adapters (Illumina) can be used for indexing. Sequencing is performed on the library using a sequencer platform (e.g., MiSeq or HiSeq, Illumina).

Bisulfite-modified DNA reads are aligned to a reference genome using alignment software (e.g., Bismark tool version 0.12.7). Differential methylation is calculated for specific loci/regions.

### Example 5. Hybrid Probe Capture

Probesets were designed to target a plurality of differentially methylated regions (DMRs) listed in Table 1. Probesets were designed using multiple methods. For some probesets, we used RRBS read data produced from pools of samples exhibiting a range of methylation states as the reference sequence for probe design. For the alternate probesets, we used an in silico simulated methylation state probe design method. Briefly, target genome regions are extracted from the reference assembly (hg38) and then bisulfite-converted versions of a variety of methylation states of both genome strands are simulated, and a portion of these were selected for probe design. Probes were then tiled across each of these simulated-converted regions at roughly 2× tiling density. Once all candidate probes were selected, they were filtered for specificity.

Extracted samples from patients and control DNA samples were run multiple times to assess inter- and intra-capture reproducibility. Extracted cfDNA was used for bisulfite treatment using the EZ DNA Methylation-Gold Kit (Zymo Research), followed by library preparation with the Accel-NGS Methyl-Seq DNA Library Kit (Swift Biosciences) and indexing amplification using unique dual 8 bp indexing primers. Yields ranged from 123 ng to 4.1 ug based on total library quantitative PCR. Each library was analyzed using a Bioanalyzer instrument (Agilent Technologies) to gauge the portion of the total library mass that likely stemmed from target genomic regions (e.g., 200 to 650 bp after library preparation), which ranged from 23 to 90%. This estimated proportion was then used to take the appropriate total library amount intended insert material to target enrichment. Eight or more libraries were pooled for each enrichment reaction, with a total library mass of up to 1.6 ug insert-containing templates. Target enrichment was carried out using baits synthesized in a commercial setting. Briefly, bisulfite-converted DNA libraries were incubated with 5′-biotinylated probes and blockers in hybridization buffer overnight at 63° C. Probe-bound libraries were pulled down with streptavidin beads followed by four 63° C. washes and amplified with 14 PCR cycles. Then, a second-round overnight hybridization was performed to achieve high target capture efficiency. The enriched libraries were quantified with KAPA Library Quantification Kit (Roche) and sequenced on a NovaSeq using 2×150 cycle runs. Several captures were also sequenced using PE75 and PE300 protocols with a MiSeq using v3 chemistry.

Paired end FASTQ files were generated on MiSeq and NovaSeq sequencers (Illumina). After demultiplexing, FASTQ quality was assessed using FastQC. Based on results from FastQC FASTQs were hard trimmed at the 3′ end from 300 bp to 100 bp. After QC, FASTQ adapter trimming was performed using TrimGalore. Read 2 FASTQs were trimmed 10 bp from the 5′ end to remove the low complexity oligonucleotide introduced by Swift Biosciences' adaptase. After trimming, paired end reads were mapped to hg38 using Brabham Bioinformatics' Bismark BS-seq alignment software. After alignment duplicate reads were removed using Samblaster. Methylation per CpG was evaluated using Bismark's methylation extractor tool. QC reports were combined using MultiQC. All downstream analysis was performed in R using the bsseq package.

While specific embodiments have been described above with reference to the disclosed embodiments and examples, such embodiments are only illustrative and do not limit the scope of the invention. Changes and modifications can be made in accordance with ordinary skill in the art without departing from the invention in its broader aspects as defined in the following claims.

All publications, patents, and patent documents are incorporated by reference herein, as though individually incorporated by reference, including U.S. Pat. Nos. 10,525,148; 11,035,849; U.S. Pat. Pub No. US 20200340062; and PCT Pat. Pub. No. WO 2020150258. No limitations inconsistent with this disclosure are to be understood therefrom. The invention has been described with reference to various specific and preferred embodiments and techniques. However, it should be understood that many variations and modifications may be made while remaining within the spirit and scope of the invention.

What is claimed is:

1. A method for determining whether a subject is likely to have or develop epithelial ovarian cancer in a subject comprising:
(a) measuring the level of nucleic acid methylation of a plurality of target genomic region listed in Table 1 from a cell-free nucleic acid sample from the subject;
(b) comparing the level of nucleic acid methylation of the plurality of target genomic region in the sample to the level of nucleic acid methylation of the plurality of target genomic regions in a sample isolated from a cancer-free subject, a cancer-free reference standard, or a cancer-free reference cutoff value;
(c) determining that the subject is like to have or develop epithelial ovarian cancer based on a change in the level of nucleic acid methylation in the plurality of target genomic regions in the sample derived from the subject, wherein the change is greater or less than the level of nucleic acid methylation of the target genomic regions in the sample isolated from a cancer-free subject, a normal reference standard, or a normal reference cutoff value.

2. The method of claim 1 wherein the method determines a presence of stage 1, stage II, stage III, or stage IV epithelial ovarian cancer of any epithelial histological subtype.

3. The method of claim 2 wherein the epithelial histological subtype is selected from the group consisting of endometrioid ovarian cancer, mucinous ovarian cancer, clear cell ovarian cancer, and serous ovarian cancer.

4. The method of claim 1 wherein the methylation level is determined using one or more of enzymatic treatment, bisulfite amplicon sequencing (BSAS), bisulfite treatment of DNA, methylation sensitive PCR, bisulfite conversion combined with bisulfite restriction analysis, post whole genome library hybrid probe capture, and TRollCamp sequencing.

5. The method of claim 4 wherein the methylation levels of the target genomic is determined using hybrid probe capture.

6. The method of claim 5 comprising one or more probes that hybridize to the one or more target genomic regions, wherein the one or more target genomic regions comprise an uracil at each position corresponding to an unmethylated cytosine in the DNA molecule.

7. The method of claim 6 wherein each of the one or more probes is configured to hybridize to:
a) a nucleotide sequence of the one or more target genomic regions comprising uracil at each position corresponding to a cytosine of a CpG site of the nucleic acid molecule; or
b) a nucleotide sequence of the one or more target genomic regions comprising cytosine at each position corresponding to a cytosine of a CpG site of the nucleic acid molecule.

8. The method of claim 6 wherein each of the one or more probes comprises ribonucleic acid, and each of the one or more probes comprises and affinity tag selected from the group consisting of biotin and streptavidin.

9. (canceled)

10. (canceled)

11. The method of claim 1 wherein the plurality of target genomic regions comprises at least 30% of the target genomic regions of Table 1.

12. (canceled)

13. (canceled)

14. The method of claim 1 wherein the plurality of target genomic regions comprises at least 60% of the target genomic regions of Table 1.

15. (canceled)

16. (canceled)

17. The method of claim 1 wherein the plurality of target genomic regions comprises at least 90% of the target genomic regions of Table 1.

18. (canceled)

19. The method of claim 1 wherein the plurality of target genomic regions comprises greater than 95% of the target genomic regions of Table 1.

20. The method of claim 1 wherein the plurality of target genomic regions exclude the genomic target regions Chr2: 38323997-38324203, Chr2: 113712408-113712611, Chr3:20029245-20029704, Chr8:58146211-58146673, Chr8:124995553-124995624, Chr9:89438825-89439085, Chr11:63664463-63664769, Chr11:120496972-120497256, and Chr20:5452392-5452552.

21. The method of claim 1 wherein the cell free nucleic acid sample is from whole blood, plasma, serum, or urine.

22. The method of claim 1 further comprising treating the epithelial ovarian cancer in the subject, wherein the treatment comprises one or more of radiation therapy, surgery to remove the cancer and, administering a therapeutic agent to the patient.

23. The method of claim 1 comprising the use of a trained machine learning algorithm to determine whether the subject is likely to have or develop the epithelial ovarian cancer.

24. The method of claim 23 wherein the machine learning algorithm comprises a Random Forest, a support vector machine (SVM), a neural network, or a deep learning algorithm.

25. The method of claim 23 wherein the trained machine learning algorithm is trained using samples comprising known epithelial ovarian cancer samples and known cancer-free ovarian and/or fallopian tubes samples, wherein the target genomic regions of Table 1 for each of the cell-free nucleic acid sample is examined for differential methylation.

26. A method for detecting high grade serous epithelial ovarian cancer in a subject comprising:
(a) measuring the level of nucleic acid methylation of a plurality of target genomic region listed in Table 1 from a cell-free nucleic acid sample from the subject;
(b) comparing the level of nucleic acid methylation of the plurality of target genomic region in the sample to the level of nucleic acid methylation of the plurality of target genomic regions in a sample isolated from a cancer-free subject, a cancer-free reference standard, or a cancer-free reference cutoff value;
(c) determining that the subject has high grade serous epithelial ovarian cancer based on a change in the level of nucleic acid methylation in the plurality of target genomic regions in the sample derived from the subject, wherein the change is greater or less than the level of nucleic acid methylation of the target genomic regions in the sample isolated from a cancer-free subject, a normal reference standard, or a normal reference cutoff value.

27. A method for differentiating high grade serous epithelial ovarian cancer from non-high grade serous epithelial cancer in a subject comprising:
(a) measuring a level of nucleic acid methylation of a plurality of target genomic region listed in Table 1 from a cell-free nucleic acid sample from the subject;
(b) comparing the level of nucleic acid methylation of the plurality of target genomic region in the sample to a level of nucleic acid methylation of the plurality of target genomic regions in a sample isolated from a non-high grade serous epithelial ovarian cancer subject.;
(c) determining that the subject has high grade serous epithelial ovarian cancer based on a change in the level of nucleic acid methylation in the plurality of target genomic regions in the sample derived from the subject, wherein the change is greater or less than the level of nucleic acid methylation of the target genomic regions in the sample isolated from a non-high grade serous epithelial ovarian cancer subject.
